(19) United States
(12) Patent Application Publication
(10) Pub. No.: US 2006/0034891 A1
(43) Pub. Date: Feb. 16, 2006

(54) Biodegradable controlled release bioactive agent delivery device

(76) Inventors: Laurie Lawin, New Brighton, MN (US); Aron B. Anderson, Minnetonka, MN (US)

Correspondence Address: KARRIE WEAVER;Kagan Binder, PLLC, Suite 200, 221 Main Street North, Stillwater, MN, 55082, US

(21) Appl. No.: 11/175,910

(22) Filed: Jul. 5, 2005

Related U.S. Application Data
(60) Provisional application No. 60/600,930, filed on Aug. 12, 2004.

(51) Int. Cl. A61F 2/00

(52) U.S. Cl. 424/427

(57) ABSTRACT

The invention provides implantable medical devices that are fabricated, at least in part, from biodegradable polymeric material. The implantable medical devices are used to provide bioactive agent to a treatment site, and are particularly useful for treatment of limited access regions of the body.

This application claims the benefit of U.S. Provisional Application Ser. No. 60/600,930, filed Aug. 12, 2004, entitled “BIODEGRADABLE MEDICAL DEVICES FOR OPHTHALMIC APPLICATIONS,” which application is incorporated herein by reference in its entirety.

### FIELD OF THE INVENTION

The invention relates to medical devices having a biodegradable component that are useful for effectively treating a treatment site within a patient's body, for example, treating limited access regions of the body, such as the eye.

### BACKGROUND OF THE INVENTION

Many surgical interventions involve placement of a medical device into the body. While beneficial for treating a variety of medical conditions, the placement of metal or polymeric devices in the body can give rise to numerous complications. Some of these complications include increased risk of infection, initiation of a foreign body response (which can result in inflammation and/or fibrous encapsulation), and initiation of a wound healing response (which can result in hyperplasia).

One approach to reducing the potential harmful effects that can result from medical device implantation is to fabricate at least a component of the device from a synthetic polymeric composition that is bioerodible. For example, surgically implantable biomaterials can serve as artificial devices introduced into living tissues to replace (prosthesis) or augment (implant) a missing part of the body. Such articles include vascular grafts, biodegradable sutures, and orthopedic appliances such as bone plates and the like. In order for an implantable or prosthetic device to be useful, it should be composed of a synthetic polymeric composition having sufficient tensile strength and elasticity over a preselected minimal time period that will vary with the specific application. The synthetic composition should also be non-immunogenic, biocompatible, biodegradable in vivo and yield degradation products that are themselves non-inflammatory, non-toxic and non-antigenic.

Another approach to reducing the potential harmful effects that can result from medical device implantation is to deliver bioactive compounds to the vicinity of the implanted device. This approach attempts to diminish harmful effects that arise from the presence of the implanted device. For example, antibiotics can be released from the device to minimize infection, and antiproliferative drugs can be released to inhibit hyperplasia. One benefit of the local release of bioactive agents is the avoidance of toxic concentrations of drugs that are sometimes necessary, when given systemically, to achieve therapeutic concentrations at the site where they are required.

Further, medical devices can include one or more bioactive agents that are to be released from the device to treat the condition, in addition to, or in place of, the bioactive agents that reduce harmful effects of the implant itself.

Several challenges confront the use of medical devices that release bioactive agents into a patient's body. For example, treatment may require release of the bioactive agent(s) over an extended period of time (for example, weeks, months, or even years), and it can be difficult to sustain the desired release rate of the bioactive agent(s) over such long periods of time. Further, the device surface is preferably biocompatible and non-inflammatory, as well as durable, to allow for extended residence within the body. Preferred devices intended for implantation in the body are manufactured in an economically viable and reproducible manner, and they are preferably sterilizable using conventional methods.

In particular, placement of implantable devices in limited access regions of the body can present additional challenges. Limited access regions of the body can be characterized in terms of physical accessibility as well as therapeutic accessibility. Factors that can contribute to physical accessibility difficulties include the size of the region to be reached (for example, small areas such as glands), the location of the region within the body (for example, areas that are embedded within the body, such as the middle or inner ear), the tissues surrounding the region (for example, areas such as the eye or areas of the body surrounded by highly vascularized tissue), or the tissue to be treated (for example, when the area to be treated is composed of particularly sensitive tissue, such as areas of the brain).

Factors that can contribute to therapeutic accessibility can be seen, for example, in the delivery of drugs to the eye. Ocular absorption of systemically administered pharmacologic agents is limited by the blood ocular barrier, namely the tight junctions of the retinal pigment epithelium and vascular endothelial cells. High systemic doses of bioactive agents can penetrate this blood ocular barrier in relatively small amounts, but expose the patient to the risk of systemic toxicity. Intravitreal injection of bioactive agents (such as drugs) is an effective means of delivering a drug to the posterior segment of the eye in high concentrations. However, these repeated injections carry the risk of such complications as infection, hemorrhage, and retinal detachment. Patients also often find this procedure somewhat difficult to endure.

An implantable medical device that can undergo flexion and/or expansion upon implantation, and that is also capable of delivering a therapeutically significant amount of a pharmaceutical agent or agents from the surface of the device has been described. See U.S. Pat. Nos. 6,214,901 and 6,344,035, published PCT Application No. WO 00/55396, and U.S. Patent Application Publication Nos. 2002/0032434, 2003/0031780, and 2002/0188037.

A therapeutic agent delivery device that is particularly suitable for delivery of a therapeutic agent to limited access regions, such as the vitreous chamber of the eye and inner ear is described in U.S. Pat. No. 6,719,750 B2, as well as U.S. Patent Application Publication No. 2005/0019371 A1, entitled “Controlled Release Bioactive Agent Delivery Device,” Anderson et al., filed Apr. 29, 2004.

### SUMMARY OF THE INVENTION

Generally, the invention provides implantable medical devices fabricated from biodegradable or bioabsorbable materials that are utilized for delivery of one or more bioactive agents to a treatment site within the body. The biodegradable component can be composed of a number of polymeric materials, which can be viewed (for purposes of discussion), as falling within one of the following general groups: non-peptide polyamino acid polymers, polyiminocarbonates, amino acid-derived polycarbonates and polyarylates, and poly(alkylene oxide) copolymers. Optionally, the biodegradable polymeric materials can be modified, for example, by inclusion of pendent carboxylic acid groups, by formation of a porous scaffold structure, and/or by inclusion of a second polymer within the polymeric matrix formed by the biodegradable polymeric material. Preferably, the biodegradable polymeric material is selected from tyrosine-derived polymers with one of two distinct backbone structures, namely polycarbonate and polyarylate backbones. Changes in the chemical structure of monomers used to form these polymers can provide polycarbonates and polyarylates with a wide range of material, chemical, and physical properties. In addition, the introduction of poly(alkylene oxide) blocks into the backbone of the polymeric material can alter material characteristics.

The invention thus provides methods and devices for controlled delivery of a bioactive agent wherein at least a portion of the device is fabricated from a biodegradable polymeric material. According to some aspects, the polymeric material degrades after implantation over a predetermined period of time so that surgical removal of the delivery device is not required, but is possible, if desired.

In a more specific aspect, the invention provides devices and methods for providing treatment (for example, of ocular structures), wherein the devices include at least a component that is biodegradable and/or bioerodible. In preferred aspects, any portions of the device that remain in the body (portions that are not degraded and/or absorbed) do not cause significant adverse foreign body response. Further, it is preferred that any portions that remain in the body do not significantly interfere with function of the body region in which the device is implanted. For example, when the device is utilized to treat the eye, it is preferred that any portions of the device that remain in the eye do not interfere with vision.

The invention provides devices and methods for providing a biodegradable implantable device for delivery of one or more bioactive agents to a treatment site within the body in a controllable manner. The invention can provide particular advantages when used to deliver bioactive agent(s) to limited access regions of the body. Preferred embodiments of the invention relate to devices and methods for providing bioactive agent(s) to treatment sites in a manner that minimizes damage and interference with body tissues and processes. A primary function of the inventive device is to deliver the bioactive agent(s) to a desired treatment site within the body, and in preferred embodiments, the device itself does not provide any other significant function. In one embodiment, for example, once the desired treatment of the body has been accomplished, the device preferably has degraded to a point where it is no longer significantly present. In another embodiment, one or more portions of the device are fabricated from a material that is biodegradable, while other portions of the device do not degrade. According to these aspects, portions of the device remain in the body and can be removed, if desired (of course, it is understood that removal is not required). Moreover, preferred embodiments of the invention provide a device that is minimally invasive such that risks and disadvantages associated with more invasive surgical techniques can be reduced.

According to the invention, bioactive agent can be released from the device via diffusion through portions of the device (for example, diffusion through the biodegradable polymeric material). Bioactive agent can be released via the degradation process itself, such that as the polymeric material degrades, the bioactive agent is released. Bioactive agent can be presented on a surface of the device in a non-releasable manner, such that treatment with the bioactive agent is effected at the surface (such as an antithrombogenic surface). Any combination of these mechanisms is also possible within the invention.

For ease of discussion, reference will repeatedly be made to a “bioactive agent.” While reference will be made to a “bioactive agent,” it will be understood that the invention can provide any number of bioactive agents to a treatment site. Thus, reference to the singular form of “bioactive agent” is intended to encompass the plural form as well.

These and other aspects and advantages will now be described in more detail.

### BRIEF DESCRIPTION OF THE DRAWINGS

The accompanying drawings, which are incorporated in and constitute a part of this specification, illustrate several aspects of the invention and together with the description of the preferred embodiments, serve to explain the principles of the invention. A brief description of the drawings is as follows:

FIG. 1 is a perspective view of an implantable device according to one embodiment of the invention.

FIG. 2 is a view from the bottom of the embodiment illustrated in FIG. 1.

FIG. 3 is a perspective view of an implantable device according to another embodiment of the invention.

FIG. 4 is a view from the bottom of the embodiment illustrated in FIG. 3.

FIG. 5 illustrates transcleral placement of an implantable device according to one embodiment of the invention.

### DETAILED DESCRIPTION OF THE INVENTION

The embodiments of the invention described below are not intended to be exhaustive or to limit the invention to the precise forms disclosed in the following detailed description. Rather, the embodiments are chosen and described so that others skilled in the art can appreciate and understand the principles and practices of the invention.

Various terms relating to the systems and methods of the invention are used throughout the specification.

Unless indicated specifically otherwise, for all molecular weight determinations, measurement is done by gel permeation chromatography (GPC) relative to polystyrene standards without further correction, or utilizing light scattering techniques.

As used herein, “biocompatible” means the ability of an object to be accepted by and to function in a recipient without eliciting a significant foreign body response (such as, for example, an immune, inflammatory, thrombogenic, or the like response). For example, when used with reference to one or more of the polymeric materials of the invention, biocompatible refers to the ability of the polymeric material (or polymeric materials) to be accepted by and to function in its intended manner in a recipient.

As used herein, “therapeutically effective amount” refers to that amount of a bioactive agent alone, or together with other substances (as described herein), that produces the desired effect (such as treatment of a medical condition such as a disease or the like, or alleviation of a symptom such as pain) in a patient. The phrase “prophylactically effective amount” likewise is an art-recognized term. In some aspects, the phrase refers to an amount of bioactive agent that, when incorporated into a biodegradable composition of the invention, provides a preventative effect sufficient to prevent or protect an individual from future medical risk associated with a particular disease or disorder. During treatment, such amounts will depend upon such factors as the particular condition being treated, the severity of the condition, the individual patient parameters including age, physical condition, size and weight, the duration of the treatment, the nature of the particular bioactive agent thereof employed and the concurrent therapy (if any), and like factors within the knowledge and expertise of the health practitioner. A physician or veterinarian of ordinary skill can readily determine and prescribe the effective amount of the bioactive agent required to treat and/or prevent the progress of the condition.

The term “implantation site” refers to the site within a patient's body at which the implantable device is placed according to the invention. In turn, a “treatment site” includes the implantation site as well as the area of the body that is to receive treatment directly or indirectly from a device component. For example, bioactive agent can migrate from the implantation site to areas surrounding the device itself, thereby treating a larger area than simply the implantation site. The term “incision site” refers to the area of the patient's body (the skin and transdermal area) at which an incision or surgical cut is made to implant the device according to the invention. The incision site includes the surgical cut, as well as the area in the vicinity of the surgical cut, of the patient.

The term “treatment course” refers to the dosage rate over time of one or more bioactive agents, to provide a therapeutically effective amount to a patient. Thus, factors of a treatment course include dosage rate and time course of treatment (total time during which the bioactive agent(s) is administered).

The invention is directed to medical devices fabricated from biodegradable polymeric material. At least a portion of the device is biodegradable, and this portion is broken down gradually by the body after implantation.

The invention is directed to methods and apparatuses for effectively treating a treatment site within a patient's body, and in particular for treating the eye and/or ocular structures. According to preferred embodiments of the invention, degradable devices are provided that can provide treatment to a site within the body for a desired period of time, during and/or after which at least a portion of the device degrades. Preferably, the inventive methods and apparatuses can be utilized to deliver bioactive agent to a treatment site as well. Such methods and apparatuses in accordance with the invention can advantageously be used to provide flexibility in treatment duration and type of bioactive agent delivered to the treatment site. In particular, the invention has been developed for controllably providing one or more bioactive agents to a treatment site within the body for a desired treatment course, and it is particularly useful for delivering bioactive agents to a limited access region of a patient's body, such as the eye, ear, spinal cord, brain, and joints.

In order to be properly introduced and utilized, implantable devices of all sorts of types are preferably designed to accommodate needs for advanceability, manipulability, and crossability to the distal end of the device as such is applied to the proximal end of the device. For purposes of this application, the following terms are given the following meaning. Advanceability is the ability to transmit force from the proximal end of the device to the distal end of the device. The body member of the device should have adequate strength for advanceability and resistance to buckling or kinking. Manipulability is the ability to navigate tortuous vasculature or other body passages to reach the treatment site. A more flexible distal portion is known to improve manipulability. Thus, it can be desirable to provide a device having a body member with some elastomeric properties to improve flexibility in some applications. Crossability is the ability to navigate the device across tissue barriers or narrow restrictions in the body.

Optimization of advanceability, manipulability, and crossability can be accomplished by carefully choosing the device material and its physical characteristics, such as thickness of the material forming the body member. Further, in order to achieve a combination of desired properties at different parts of the device itself, the device can be fabricated to combine a plurality of components together to define a device body member. That is, a portion of the overall length of a body member of the device can comprise a different component than another. These one or more portions can comprise components of different physical characteristics and/or different materials. For example, a distal tip portion can be provided that is more resilient than the remainder of the device body member for better crossability and to provide a softer leading end of the device for abutting body internal membranes and the like. Different materials include different metallic materials or polymeric materials from one another, for example, or similar polymers of different densities, fillers, crosslinking, degradation rates, or other characteristics. In particular, a portion of a device body member can comprise a material chosen for flexibility to allow flexion of the device during residence within the body (for example, in such areas as joints, where movement of the tissues in the area is likely) while another portion can comprise a material chosen for axial and/or torque transmission (to assist in placement of the device).

Further, the specific portions of the device comprising biodegradable polymeric material can be chosen depending upon the end use of the device. According to these aspects, the device material can be chosen with regard to the function of portions of the device. For example, when a proximal portion of the device is configured to anchor the device in place during treatment, this proximal portion can be fabricated of a non-degradable material or a biodegradable polymeric material that degrades more slowly than the remainder of the device. In this particular embodiment, the proximal anchor portion can serve its anchoring function for the period of time during which the distal portion of the device provides its corresponding function (such as delivery of bioactive agent). One specific illustration of these aspects of the invention can be envisioned wherein the body member includes a cap, as described later herein. The body member can be fabricated of a biodegradable polymeric material, while the cap can be fabricated of a nondegradable material (such as metal, polymer, and the like) or a biodegradable polymeric material that degrades more slowly than the biodegradable polymeric material of the body member. The cap can thus provide anchoring function for a desired time, while the degradable portion functions for bioactive agent delivery. Any combination of degradable/non-degradable portions, as well as portions with varying rates of degradation, of the device can be provided utilizing the teaching herein.

According to the invention, a device has been developed that can be used to treat any implantation site within the body in which it is desirable to provide controlled release of one or more bioactive agents. In preferred embodiments, the device can be used to provide one or more bioactive agents to a treatment site that comprises a limited access region of the body, such as the eye, ear, brain, spine, and joints.

To facilitate the discussion of the invention, use of the invention to treat an eye will be addressed. Eyes are selected as a result of the particular difficulties encountered when treating medical conditions of the eye, as described herein. Further, in terms of lowering the risk of damage to body tissues while providing a superior device, the advantages of this controlled release device can be clearly presented. However, it is understood that the device and methods disclosed are applicable to any treatment needs, for example, treatment of limited access regions of the body where controlled release of a bioactive agent is desired during treatment, such as, for example, the central nervous system (the brain and spinal cord), the ear (such as the inner ear), and joints.

The invention provides biodegradable devices for controlled delivery of bioactive agent to a treatment site. According to the invention, at least a portion of the device is composed of a biodegradable polymeric material.

Biodegradable Polymeric Materials

The invention provides implantable devices that are useful for treatment of limited access regions of the body. At least a portion of the implantable device is fabricated from a biodegradable polymeric material. In some embodiments, the entire implantable device can be fabricated from one or more types of biodegradable polymeric materials. Suitable biodegradable polymeric materials are selected from non-peptide polyamino acid polymers, polyiminocarbonates, amino acid-derived polycarbonates and polyarylates, and poly(alkylene oxide) copolymers. Each of these polymeric materials will now be described.

The biodegradable polymeric materials described herein are particularly advantageous for treatment of limited access regions of the body. The biodegradable polymeric materials described herein can be readily adapted to include one or more bioactive agents for such treatment. This provides flexibility in treatment regimens. Additionally, the biodegradable polymeric materials break down to form degradation products that are non-toxic and do not cause a significant adverse reaction from the body. Thus, the inventive methods and devices provide the ability to treat limited access regions of the body with a desired bioactive agent or agents, while maintaining the environment of the body region being treated. In other words, neither the amount nor the presence of the degradation products and/or bioactive agent significantly interfere with function and/or integrity of the treatment site. This is particularly advantageous in regions of the body that are relatively isolated from other portions of the body (for example, the eye, which is a relatively small environment that is difficult to access due to the blood/ocular barrier). As some regions of the body are not flushed, or are very slowly flushed, with bodily fluids (such as blood or the like), clearance of degradation products and/or bioactive agent can be a significant barrier to treatment with known methods. As will be apparent from the discussion herein, preferred embodiments of the invention can overcome such barriers.

I. Non-Peptide Polyamino Acid Polymers

In one aspect, the biodegradable polymeric material is composed of a non-peptide polyamino acid polymer. Suitable non-peptide polyamino acid polymers are described, for example, in U.S. Pat. No. 4,638,045 (“Non-Peptide Polyamino Acid Bioerodible Polymers,” Jan. 20, 1987). Generally speaking, these polymeric materials are derived from monomers, comprising two or three amino acid units having one of the following structures illustrated in Formulae 1 and 2: 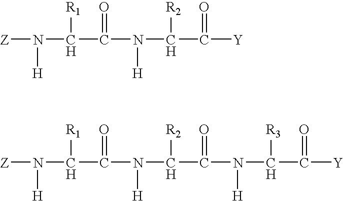
wherein the monomer units are joined via hydrolytically labile bonds at not less than one of the side groups R₁, R₂, and R₃, and where R₁, R₂, R₃ are the side chains of naturally occurring amino acids as described in Table 1 (below); Z is any desirable amine protecting group or hydrogen; and Y is any desirable carboxyl protecting group or hydroxyl. Each monomer unit comprises naturally or non-naturally occurring amino acids that are then polymerized as monomer units via linkages other than by the amide or “peptide” bond. The monomer units can be composed of two or three amino acids united through a peptide bond and thus comprise dipeptides or tripeptides. Regardless of the precise composition of the monomer unit, all are polymerized by hydrolytically labile bonds via their respective side chains rather than via the amino and carboxyl groups forming the amide bond typical of polypeptide chains. Such polymer compositions are nontoxic, are biodegradable, and can provide zero-order release kinetics for the delivery of bioactive agents in a variety of therapeutic applications.

According to these aspects, the amino acids are selected from the approximately 20 naturally occurring L-alpha amino acids whose side chains fall into different structural groups and provide a diversity of function. These L-alpha amino acids and their side groups provide at least the following functional variations: lipophilic or nonpolar groups such as the side chains of alanine, valine, leucine, isoleucine, and proline; polar or hydrophilic groups such as the side chains of serine, threonine, aspartic acid, glutamic acid, asparagine, glutamine, lysine, hydroxylysine, arginine, hydroxyproline, and methionine; groups capable of oxidation-reduction such as those of cysteine or cystine; groups having pi-bonded or aromatic character such as those of phenylalanine, tyrosine, tryptophan, and histidine; and positively or negatively charged side chains such as those of aspartic acid, glutamic acid, lysine, hydroxylysine, arginine, and histidine. In addition to these, a number of amino acids are also useful, including citrulline, ornithine, lanthionine, hypoglycin A, beta-alanine, gamma amino butyric acid, alpha aminoadipic acid, canavanine, venkolic acid, thiolhistidine, ergothionine, dihydroxyphenylalanine, and others (including non-naturally occurring amino acids) well recognized and characterized in protein chemistry.

An illustrative listing of amino acids and corresponding R groups is provided in Table 1, wherein the base structure for all listed amino acids is understood to be ⁺H₃N—C(COO⁻)—H:

TABLE 1

| Amino Acid | R group | Amino acid | R group |
|---|---|---|---|
| alanine | —CH₃ | cysteine | 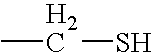 |
| valine | 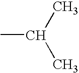 | asparagine | 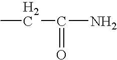 |
| leucine | 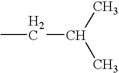 | glutamine | 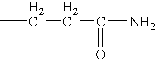 |
| proline | 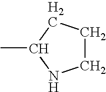 | aspartic acid | 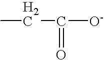 |
| phenylalanine | 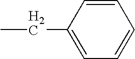 | glutamic acid | 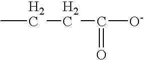 |
| tryptophan | 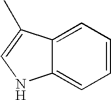 | lysine | 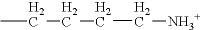 |
| methionine | 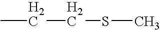 | arginine | 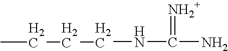 |
| glycine | —H | histidine | 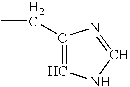 |
| serine | 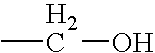 | isoleucine | 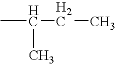 |
| threonine | 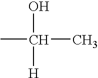 | | |

The monomer unit is preferably used with a variety of protecting groups Z and Y that are bonded to the amino and carboxyl termini, respectively, to form the monomer units seen in Formulae 1 and 2. In addition, Z can be hydrogen and Y can be hydroxyl.

The protecting groups Z can be selected freely from a large variety of biocompatible, nontoxic molecules such as fatty acids, benzoic acid, acetic acid, and the like. The protecting groups Y can be selected freely from a large variety of biocompatible, nontoxic molecules such as alcohols, amines, and the like. Methods and reactions for the joining of such protective groups at the Z and Y positions of the dipeptide monomer unit are described, for example, in Hofmann and Katsoyannis, The Proteins (2^{nd} edition) Academic Press, New York 1963; Greenstein and Winitz, Chemistry of the Amino Acids, John Wiley and Sons, New York, 1961; Hofmann and Yajima, Polyamino Acids, Polypeptides, and Proteins (M. Spahmann editor), University of Wisconsin Press, Madison, 1962.

Alternatively, the protecting groups Z and Y can in fact be bioactive agents that can be linked to the dipeptide monomer at the Z and Y positions. Such bioactive agents include those described herein, as well as other reactants that are capable of being released into the body by bioerosion of the polymerized composition. Regardless of whether bioactive agents or other kinds of organic compositions are used as the protecting groups Z and Y, the variety and choices of L-alpha amino acids comprising the monomer units (as well as the protecting groups) provide for an overall variation of chemical and mechanical properties of the polymerized composition that can be modified to accommodate the particular application.

The monomer unit containing the groups Z and Y is then polymerized by reactions that occur at not less than one of the respective side groups, R₁, R₂, and R₃. These linkages are hydrolytically labile bonds other than the amide bond, the precise nature of which will vary with the chemical formulation and structure of the respective side chain R₁, R₂, and R₃. In its simplest form, the dipeptide is formed of two identical amino acids in which the side chain R is identical in each molecule as shown in Diagram 1A and 1B below:

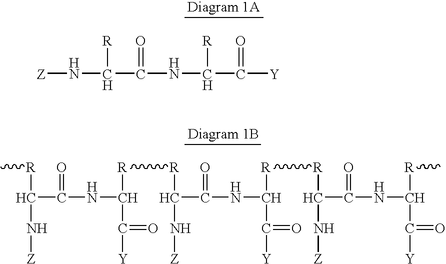

As noted, Diagram 1A is a schematic representation of a monomer unit based upon L-alpha amino acids. It will be noted that the side chains R of the dipeptide monomer unit are not part of the amide bond backbone. Diagram 1B is a schematic representation of the polymerization product of many monomer units using “non-peptide” bonds between the respective side chains R of each monomer unit. The letters Z and Y represent amino and carboxyl protecting groups respectively. The “ [symbol] ” notation symbolizes a hydrolytically unstable bond that is biodegradable. Through this series of polymerization reactions between the side groups R of each individual monomer unit, a polymeric backbone is formed that provides both the mechanical and chemical characteristics of the polymer composition as a whole.

In one illustrative embodiment, poly(N-carbobenzoxytyrosyltyrosine ethylester iminocarbonate) (hereafter CbzTyrTyrOEt iminocarbonate) is prepared according to the synthetic scheme illustrated in Diagram 2: 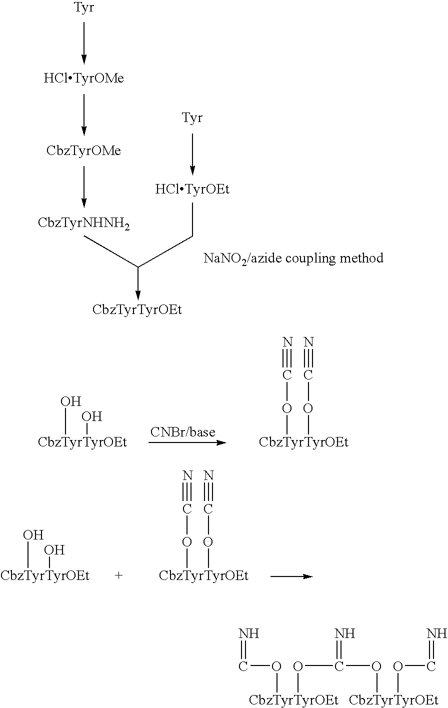
In the first reaction step illustrated in Diagram 2, tyrosine is converted to tyrosyl methyl ester or ethyl ester hydrochloride respectively. After formation of the dipeptide, the dipeptide is subsequently cyanylated to yield the corresponding dicyanate derivatives. When equimolar quantities of dipeptide monomer and dipeptide-dicyanate monomer are mixed as shown in Diagram 2, rapid polymerization occurs in the presence of a basic catalyst (such as sodium hydroxide, triethylamine, or potassium tert-butoxide) to yield the corresponding polyiminocarbonate having the general formula:
(C₂₉H₂₉N₃O₇)ₙ
In this exemplary embodiment, the carbobenzoxy group represents the protecting group Z at the amino terminal end of the monomer unit. Alternatively, suitable bioactive agents can be substituted for the carbobenzoxy group. Using known methods of chemically linking bioactive agents (such as drugs) to the amino terminal of the monomer unit, bioactive agent-monomer conjugates can be obtained that, upon polymerization, create a bioerodible polymeric composition that delivers drugs, antibiotics, hormones, and other active agents for therapeutic purposes. All that is required is that the bioactive agent molecule contains a moiety such as carboxyl that is reactive with the amino moiety or that can be modified to create a moiety that is reactive with the amino moiety. Similarly, bioactive agent-monomer conjugates can be formed at the carboxyl terminal of the monomer.

The preparation of the polyiminocarbonate polymer as described above is only one example of the many different kinds of polymers derived from monomer units whose side chains are linked by hydrolytically labile bonds. In other embodiments, the amino acids forming the monomer unit are different thereby giving rise to different and independent side groups R₁, R₂, and R₃ that are joined to one another between monomer units. Other useful examples in a non-exhaustive listing are given in Table 2.

TABLE 2

| Compound | Bond type |
|---|---|
| Z-Tyr-Tyr-Y | Iminocarbonates |
| Z-Glu-Glu-Y | Anhydrides |
| Z-Glu-Phe | Anhydrides |
| Z-Tyr-Glu-Y | Phenyl esters |
| Z-Tyr-Phe | Phenyl esters |
| Z-Ser-Phe | Aliphatic esters |
| Z-His-Phe | Imidazolides |
| Z-His-Glu-Y | Imidazolides |
| Z-Cys-Cys-Y | Sulfides |

In these examples, blocking groups Z and Y are presumed to be known in the art, and the choice of specific functional groups at either the amino or carboxyl terminal ends is a matter of convenience for the user.

In another embodiment, the monomer unit is formed using three amino acids joined together by a series of amide bonds. A basic structural formulation for such monomer units is given by the chemical Formula 2:

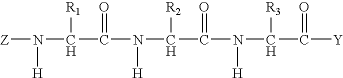

It will be recognized that this monomer structure has three side groups per unit, R₁, R₂, and R₃, for reaction via hydrolytically labile bonds to form the polymeric composition. It is not required, however, that all three side chains be actively involved in the polymerization process. In most instances, only two of the three side chains R₁, R₂, and R₃ will be involved in reactions to form the polymer. This is schematically represented in Diagram 3, wherein the tripeptide monomer units form a polymer by hydrolytically labile bonds between the R₁ and R₃ side groups between individual monomer units. As before, the Z and Y compositions represent amino and carboxylprotecting groups, respectively, and the “ [symbol] ” notation symbolizes a hydrolytically unstable bond.

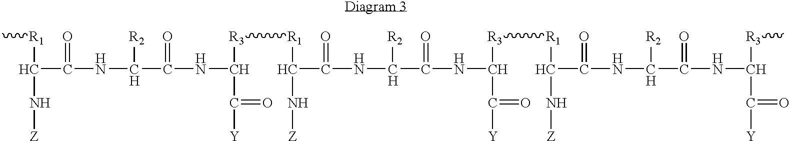

The minimum number of side chains in the monomer composition that must be involved in the polymerization process via hydrolytically labile bonds, however, is only one. In this instance, either the Z and/or Y protecting groups are omitted from the monomer unit, leaving a functional amino and/or carboxyl group intact for reaction via a non-amide bond (a hydrolytically labile bond), with the reactive side group R of another monomer unit. This kind of monomer unit and polymerization reaction is schematically illustrated in Diagram 4A and 4B:

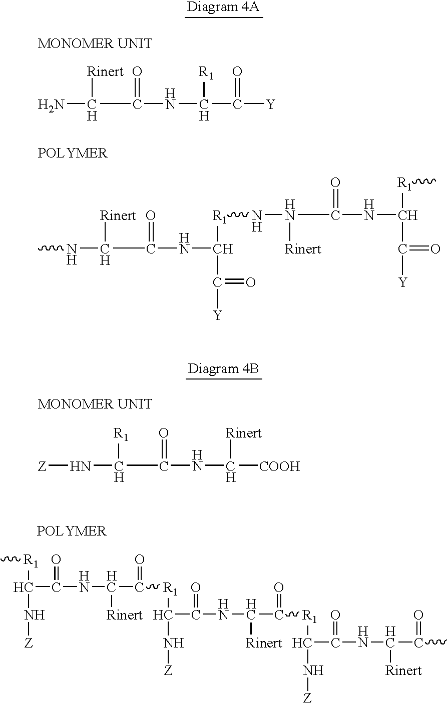

It will be apparent to one skilled in the art, upon review of the disclosure, that other side groups R than those listed herein are available for reaction with the amino terminal end or carboxyl terminal end of monomer units under similar conditions using known methods of reaction. Although such polymeric compositions are more structurally and chemically complex compared to those previously described, all such polymeric compositions wherein at least one of the side groups in the monomer unit are joined via hydrolytically labile bonds to another monomer unit can be used as described herein.

As described herein, a bioactive agent can be chemically incorporated into the polymer chains as pendent side chains. Alternatively, a polymeric matrix of monomers can be prepared and any bioactive agent can be physically embedded or dispersed within the polymeric matrix. The chemical, mechanical, and biodegradability properties are adjustable and will vary with the number and kind of amino acids comprising the monomer unit and the nature of the hydrolytically labile bond.

II. Polyiminocarbonates

In addition to the non-peptide polyamino acid polymers described above, the biodegradable polymeric material can be composed of polyiminocarbonates. Polyiminocarbonates are structurally related to polycarbonates, wherein imino groups are present in the places normally occupied by carbonyl oxygen in the polycarbonates. Thus, the biodegradable component can be formed of polyiminocarbonates having linkages according to the Formula 3: 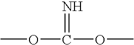
These linkages impart a significant degree of hydrolytic instability to the polymer. The polyiminocarbonates also have desirable mechanical properties akin to those of the corresponding polycarbonates. These aspects will now be described in more detail.

One class of polyiminocarbonates is described, for example, in U.S. Pat. No. 4,806,621 (“Biocompatible, Bioerodible, Hydrophobic, Implantable Polyimino Carbonate Article,” Feb. 21, 1989). According to these aspects, a device can be fabricated, at least in part, of a polyiminocarbonate composition having the general Formula 4:

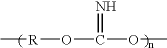

wherein R contains a non-fused aromatic organic ring, and n is greater than 1. Preferred embodiments of the R group within the general Formula 4 above is exemplified by, but is not limited to, groups listed in Table 3 below:

TABLE 3
R group

| | |
|---|---|
| (a) | 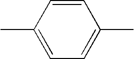 |
| (b) | 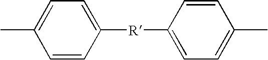 |
| | wherein R′ is lower alkene C₁ to C₆ |
| (c) | 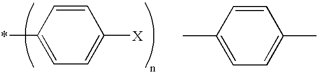 |
| | wherein n is an interger equal to or greater than 1, X is a hetero |
| | atom such as —O—, —S—, or a bridging group such as —NH—, —S(═O)—, |
| | —SO₂—, —C(═O)—, —C(CH₃)₂—, —CH(CH₃)—, —CH(CH₃)—CH₂—CH(CH₃)—, |
| (d) | 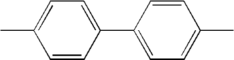 |

Also, compounds of the general Formula 5 can be utilized: 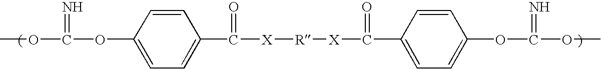
wherein X is O, NH, or NR′″, wherein R′″ is a lower alkyl radical; and R″ is a divalent residue of a hydrocarbon including polymers such as polyolefins, for example, an oligoglycol or polyglycol such as polyalkylene glycol ether, a polyester, polyurea, polyamine, polyurethane, or polyamide.

The polyiminocarbonates according to these embodiments can be synthesized using alternative methods of polymerization known in the art, including bulk polymerization, solution polymerization, and interfacial polymerization.

The polyiminocarbonates degrade into residues or moieties that are themselves biocompatible and non-toxic. In preferred embodiments, the polymeric material includes bioactive agent. The relative proportions of the composition to be released to form the two-phased system can be modified over a wide range depending upon the bioactive agent to be administered and/or the desired effect. Generally, the bioactive agent can be present in an amount that will be released over controlled periods of time, according to predetermined release rates, which rates are dependent upon such factors as initial concentration of the bioactive agent in the polymeric material, the rate of diffusion of bioactive agent from the polymeric material, and the rate of erosion of the polyiminocarbonate. Proportions suitable for the purposes of these embodiments can range from about 0.01 to about 50 parts by weight of the bioactive agent to between about 99.99 to about 50 parts by weight of the polymeric material.

The polymeric material can be admixed intimately with the bioactive agent in any convenient manner, preferably by mixing the components as powders and subsequently forming the mixture into a desired shape such as by thermal forming at a temperature less than that at which the composition will become degraded and at which the polymer has desired morphological properties. In some embodiments, for example, the polymeric material can be provided in an appropriate solvent, thereby forming a casting solution. A known amount of the bioactive agent is then mixed with the casting solution, and the solution charged into a mold. The mold is then dried to remove the solvent, usually under vacuum, causing the polymer to precipitate and forming the matrix with the bioactive agent therein. Alternatively, the polymeric material in the form of a powder can be admixed with the bioactive agent in the form of a powder, and then molded under adequate temperature and pressure to the desired shape, through injection, compression or extrusion. After the polymeric matrix containing the bioactive agent is implanted in the body, it erodes by hydrolysis thereby releasing the bioactive agent.

Other suitable polyiminocarbonates, and methods of synthesizing such polyiminocarbonates, are described, for example, in U.S. Pat. No. 4,980,449 (“Polyiminocarbonate Synthesis,” Dec. 25, 1990), U.S. Pat. No. 5,140,094 (“Polyiminocarbonate Synthesis,” Aug. 18, 1992), and U.S. Pat. No. 5,264,537 (“Polyiminocarbonate Synthesis,” Nov. 23, 1993). For example, suitable polyiminocarbonates include one or more recurring structural units represented by the Formula 6: 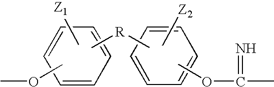
According to this structural formula, Z₁ and Z₂ can each represent one or more of the same or different radicals selected from the group consisting of hydrogen, halogen, lower-alkyl, carboxyl, amino, nitro, thioether, sulfoxide, and sulfonyl. Preferably, Z₁ and Z₂ are hydrogen. Preferably, R is selected from alkylene, arylene, alkylarylene, or a divalent functional group containing heteroatoms.

Preferred polyiminocarbonates include higher molecular weight iminocarbonates, as these higher molecular weight polymers generally provide better mechanical properties. Thus, useful polyiminocarbonates are those compounds having molecular weights above about 60,000 daltons, preferably above about 70,000 daltons, or in the range of about 100,000 to about 200,000 daltons. In yet another aspect, the polyiminocarbonate comprises a dipeptide-based polyiminocarbonate, having repeating units according to the structural formula 7: 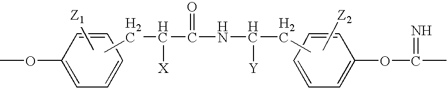
having a weight average molecular weight above about 20,000 daltons. Z₁ and Z₂ are as described above; X and Y are defined below.

For formation of polyiminocarbonates, diphenol and/or dicyanate compounds are used as starting materials. Suitable diphenol and dicyanate compounds include those disclosed in U.S. Pat. No. 3,491,060 (“Polyimidocarbonic Esters and Their Preparation,” Jan. 20, 1970). Briefly, the dicyanates described are of the formula R(OCN)₂ wherein R is an aromatic, araliphatic or heterocyclic radical. Preferred dicyanates for use herein have their —OCN groups attached to an aromatic ring system.

Particularly preferred starting material for use in accordance with these embodiments include diphenol compounds with the Formula 8: 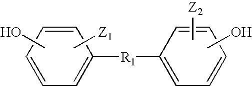
and dicyanate compounds with the Formula 9: 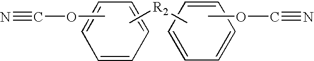
with R₁ and R₂ being the same or different and being alkylene, arylene, alkylarylene or a functional group containing heteroatoms. Z₁, and Z₂ can each represent one or more of the same or different radicals selected from the group consisting of hydrogen, halogen, lower-alkyl, carboxyl, amino, nitro, thioether, sulfoxide, and sulfonyl. Preferably, each of Z₁, and Z₂ are hydrogen.

More preferred are diphenol and dicyanate compounds in which R₁ and R₂ are selected from the group consisting of compounds shown in Formula 10: 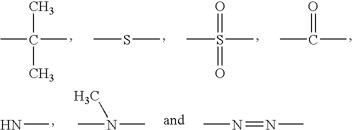
with the proviso that when R₁ is —N═N—, the diphenol compound is a meta-diphenol compound. Particularly preferred starting materials include Bisphenol A and Bisphenol A dicyanate.

Another class of particularly preferred starting materials includes peptide-derived diphenol and dicyanate compounds in which R₁ and R₂ are polyamino acids such as those disclosed in U.S. Pat. No. 4,638,045 and discussed above. Preferred peptide derived diphenol and dicyanate compounds include those in which R₁ and R₂ are compounds represented in Formula 11: 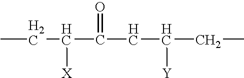
wherein X is: 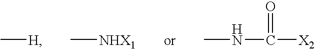
with X₁ being any one of the commonly used N-terminus protecting groups used in peptide synthesis (for example, including those described in Bodanski, Methods in Peptide Synthesis, Springer Verlag, New York, 1983). Preferred N-terminus protecting groups include: 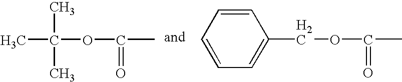
In formula 11, X₂ is a straight or branched alkyl chain; and Y is 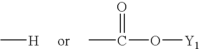
Y₁ being an alkyl, aryl, or alkylaryl radical, or any commonly used C-terminus protecting group as also disclosed by Bodanski (supra).

More preferable polyamino acid derived diphenol and dicyanate compounds include compounds in which R₁ and R₂ are selected from compounds represented in Formula 12:

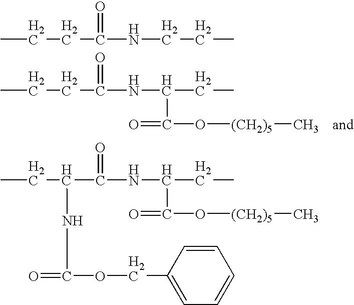

The polyiminocarbonates can be synthesized utilizing a solution polymerization process. A solution polymerization process according to this aspect includes the steps of contacting a diphenol with a dicyanate in solution in an essentially pure solvent in the presence of a catalyst selected from the group consisting of metal hydroxides, metal hydrides, and metal alkoxides, and recovering the resulting polyiminocarbonate. The solvent preferably is selected from the group consisting of acetone and tetrahydrofuran (THF). Preferably, the solvent is freshly distilled THF.

The catalyst preferably is an alkali metal hydroxide or alkoxide, such as sodium hydroxide or potassium tert-butoxide. Preferably, the catalyst is a strong base catalyst selected from metal alkoxides and metal hydroxides. Preferred metal alkoxides include sodium ethoxide and potassium tert-butoxide. Sodium hydroxide is a preferred metal hydroxide. Potassium tert-butoxide is a particularly preferred catalyst.

In preparing the solution for polymerization, solvent purity is important. The solvent used for the solution polymerization process should be essentially pure, that is, free of any impurities that would adversely affect the polymerization reaction. In particular, the solvent should be free of water and peroxides. THF used in the process should be redistilled over sodium/benzophenone immediately prior to use. Preferably, the reaction is conducted in a vessel isolated from oxygen and water vapor. Desirably, the reaction vessel is purged with dry nitrogen or argon, and the freshly distilled solvent is added by syringe. Equimolar quantities of diphenol and dicyanate should be used. The total solution concentration (w/v %) of both compounds combined typically is in the range of about 20% to about 50%, depending upon monomer solubility.

When R₁ and R₂═ 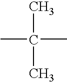
at 23° C., in the presence of up to about 1.00 mole percent solution concentration of either potassium tert-butoxide or sodium hydroxide, over 99% of the dicyanate monomer is consumed within 4 hours. Increasing the reaction time beyond 4 hours has no beneficial effect and can actually result in a reduction of polymer molecular weight.

When R₁ and R₂═ 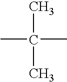
at 23° C., maximum molecular weight is obtained with either potassium tert-butoxide or sodium hydroxide at a solution concentration of 0.20 mole percent. Concentrations of potassium tert-butoxide as low as 0.05 mole percent are effective but require longer reaction times and result in lower molecular weight polymers. Concentrations of potassium tert-butoxide as high as 1.00 mole percent and sodium hydroxide as high as 1.50 mole percent are also effective and result in shorter reaction times, but also produce lower molecular weight polymers.

In preferred aspects, the reaction temperature should not exceed the range of thermal stability of the polyiminocarbonate, which, when R₁ and R₂═ 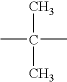
for example, is about 140° C. The reaction temperature should be higher than the solution freezing point. A preferred reaction temperature range is about 10° C. to about 78° C., the reflux temperature of THF. Most preferred is a reaction temperature of about room temperature (about 20° C. to about 30° C.). By increasing reaction temperature, reaction time is shortened. The reaction typically goes substantially to completion within about 4 hours at about 20° C. to about 30° C. Desirably, the product polymer is recovered promptly after completion of the reaction.

Optionally, reaction time can be shortened by increasing the catalyst concentration. This can be desirable when, due to the electron withdrawing nature of particular groups R₁ and R₂, reaction rates decrease, lengthening the reaction time.

The catalyst should be added all at once to the diphenol-dicyanate solution with agitation. Because polyiminocarbonates are completely soluble in THF, a clear, viscous solution forms where the solvent is THF. The polymer is recovered by evaporating the solvent from this solution, and by washing as with excess acetone.

As mentioned, the solution polymerization process can also be conducted in acetone solvent. With an acetone solvent, the catalyst desirably is sodium hydroxide at a solution concentration in the range of about 0.20 mole percent to about 1.50 mole percent, or potassium tert-butoxide catalyst of a solution concentration in the range of about 0.05 to about 1.00 mole percent. The same total solution concentrations of diphenol and dicyanate can be used as with THF.

The reaction time in acetone solvent does not significantly vary from the reaction time in THF, except that lower concentrations of potassium tert-butoxide in acetone result in a somewhat slower reaction time than the same concentrations in THF.

For R₁ and R₂═ 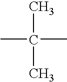
at 23° C., maximum molecular weight is obtained with potassium tert-butoxide at a solution concentration of 0.29 mole percent. For sodium hydroxide, the maximum molecular weight is obtained at a solution concentration of 1.00 mole percent. Lower and higher concentrations of catalysts also result in lower molecular weights with reaction times decreasing as catalyst is increased.

In acetone-based polymerization, the above reaction temperatures and preferred temperature ranges described with respect to THF can be used. Increasing the reaction temperature, the catalyst concentration, or both, may be used to counter any decrease in reaction rate and lengthening of reaction time caused by the electron-withdrawing nature of particular R₁ and R₂ groups.

The catalyst is preferably added to the diphenol-dicyanate acetone solution as described above with respect to THF-based polymerization. Polyiminocarbonates are not soluble in acetone and within minutes following the initiation of the catalyst addition, a polymer gel will start to separate from the reaction mixture. Upon termination of the reaction, the polymer can be separated by any convenient mechanical liquid/solid separation step (such as, for example, filtration). The separated polymer can be purified by washing in excess acetone and drying.

In alternative embodiments, polyiminocarbonates are synthesized utilizing interfacial polymerization processes. In one illustrative interfacial polymerization method, polymerization involves admixing an aqueous solution of a diphenol and a basic catalyst with a solution of cyanogen bromide in water-immiscible organic solvent by progressively adding the aqueous solution to the solution of cyanogen bromide in organic solvent while mixing, and recovering the resulting polyiminocarbonate. According to this embodiment, both the order and rate of addition are highly significant in the formation of polyiminocarbonates.

According to these embodiments, an aqueous solution containing one or more diphenol starting materials together with reaction catalyst is added slowly, with vigorous stirring into a solution of cyanogen bromide in water-immiscible organic solvent. The cyanogen bromide reacts with the diphenol to produce dicyanate, which then reacts with the remaining diphenol to form polyiminocarbonates.

The concentration of cyanogen bromide in the organic phase typically is about 0.01 to about 0.05 g/ml, and desirably about 0.03 g/ml. The concentration of diphenol in the aqueous phase typically is in the range of about 0.05 to about 0.4 molar, and desirably about 0.2 molar. The molar ratio of cyanogen bromide to diphenol added as starting materials typically is in the range of about 1:1 to about 2:1, and desirably about 1.54:1. The molar ratio of metal hydroxide reaction catalyst to diphenol can be in the range of about 0.5:1 to about 2:1, and desirably about 1:1.

The aqueous solution should be slowly added to the organic solution of cyanogen bromide over a period of at least about 60 minutes, and desirably about 120 minutes, with vigorous agitation. This agitation should be continued for at least about 60 minutes after the end of the addition, and desirably for at least about 120 minutes. Ordinarily, the polymer precipitates and is recovered by filtration and washing.

A second illustrative interfacial polymerization method involves intimately admixing an aqueous solution of a diphenol and a basic catalyst such as a metal hydroxide with a solution of a dicyanate in a water-immiscible organic solvent and recovering the resulting polyiminocarbonate. There is some hydrolysis of the dicyanate monomer in the process according to this embodiment. This results in the depletion of dicyanate and generation of diphenol. Thus, some of the diphenol used during the process is formed in situ by hydrolysis of the dicyanate. Adjusting the ratio of total diphenol added to the reaction system as a starting material before or during the reaction to total dicyanate added to the system as a starting material before or during the reaction compensates for the hydrolysis. The rate of hydrolysis varies with the solvent used; therefore the appropriate ratio of reactants will also vary accordingly. For CCl₄ solvent, the molar ratio of total diphenol to total dicyanate is preferably in the range of about 0.05:1 to about 1.00:1, or in the range of about 0.60:1 to about 0.90:1. A molar ratio of 0.83:1 is preferred. For CH₂Cl₂ solvent, the corresponding molar ratio is preferably in the range of about 0.05:1 to about 1.10:1, or in the range of about 0.80:1 to about 1.00:1. A molar ratio of about 1.00:1 is preferred.

In this interfacial polymerization method, the aqueous phase includes a diphenol and a strong base catalyst. The organic phase includes a dicyanate starting material as described herein, dissolved in a water immiscible solvent.

The aqueous solution of diphenol, strong base catalyst, and, optionally, a phase transfer catalyst (“PTC,” described in more detail below) typically is added progressively to dicyanate dissolved in water immiscible organic solvent. The progressive addition typically takes place over a period in the range of about 10 minutes to about 60 minutes, and desirably about 20 minutes. As the aqueous solution is added, the two phases are intimately admixed to bring the diphenol, dicyanate, and catalyst into reactive contact. This can be accomplished by vigorous mixing, such as by mechanical agitation or other conventional liquid-liquid contacting techniques. Upon thorough mixing of the two phases, a polyiminocarbonate precipitate forms. The precipitate can be separated by mechanical separation (for example, filtration), and purified by solvent washing.

In preferred interfacial polymerization processes, the concentration of the diphenol in the aqueous phase can typically be in the range of about 0.01 to about 1.0 molar, and preferably about 0.1 molar.

Preferred basic reaction catalysts according to this embodiment include the alkali metal hydroxides, and particularly sodium hydroxide. In this polymerization embodiment, 2 moles of the hydroxide reaction catalyst desirably are present per mole of diphenol in the aqueous phase. The aqueous phase preferably includes a PTC.

A third illustrative interfacial polymerization method involves intimately admixing an aqueous solution of a basic catalyst such as a metal hydroxide with a solution of a dicyanate in a water-immiscible organic solvent and recovering the resulting polyiminocarbonate. In this interfacial polymerization method, the dicyanate compounds described above are hydrolyzed by the catalyst to generate the diphenol for the process. Thus, no diphenol need be included in the aqueous phase as a starting material.

According to this illustrative method, the aqueous phase includes a hydroxide reaction catalyst in water, preferably together with a PTC. The organic phase includes the dicyanate. The catalyst is permitted to hydrolyze dicyanate to diphenol, which then reacts with the remaining dicyanate. The reaction conditions, including dicyanate concentration, addition times, and the like can be similar to those discussed herein in connection with the second illustrative interfacial polymerization method. Preferably, about 1 mole to about 2 moles of hydroxide reaction catalyst is provided in the aqueous phase for each mole of dicyanate in the organic phase.

In interfacial polymerization according to any of the above three illustrative methods discussed above, the reaction rate, yield, and product molecular weight can be significantly increased by adding a phase transfer catalyst (PTC) to the system, as by incorporating the PTC in the aqueous solution. PTC's are salt-like molecules that serve to transfer reactants between the aqueous and organic phases in an interfacial polymerization. The mechanisms by which PTC's function to transfer reactants, as well as numerous examples of PTC's suitable for the reaction system described herein are well known and will not be described in further detail herein. Suitable PTC's include tetrabutyl ammonium bromide (TBAB), and N-ethyl-4-dimethylamino pyridine. The concentration of PTC can be determined utilizing known techniques in view of the teaching herein. In some embodiments, for example, TBAB concentrations as high as about 50 mole percent can be utilized, while lower concentrations can increase reaction rate, molecular weight, and polymer yield. For example, the most marked increase in reaction rate, molecular weight, and polymer yield occurs for TBAB concentrations up to about 5 mole percent. Significant improvement continues between about 5 mole percent and about 10 mole percent TBAB. While reaction rate and polymer yield continues to increase beyond 10 mole percent TBAB concentration, the higher concentrations lead to a reduction of the molecular weight. The particular concentration of PTC can be determined based upon the desired polymer yield, reaction rate, and molecular weight.

III. Amino Acid-Derived Polycarbonates and Polyarylates

In addition to the non-peptide polyamino acid polymers and polyiminocarbonates described above, the biodegradable polymeric material can be composed of various types of amino acid-derived polycarbonates and polyarylates. In some aspects, the polymeric material can comprise an amino-acid derived polyarylate. In one particular embodiment, the polymeric material comprises a polyarylate derived from the natural amino acid L-tyrosine and biocompatible dicarboxylic acids. These aspects will now be described in more detail.

Suitable polyarylates, and methods for synthesis of such polymers, are described in U.S. Pat. No. 5,099,060 (“Synthesis of Amino Acid-Derived Bioerodible Polymers,” Mar. 24, 1992), U.S. Pat. No. 5,198,507 (“Synthesis of Amino Acid-Derived Bioerodible Polymers,” Mar. 30, 1993), U.S. Pat. No. 5,216,115 (“Polyarylates Containing Derivatives of the Natural Amino Acid L-Tyrosine,” Jun. 1, 1993), U.S. Pat. No. 5,317,077 (“Polyarylates Containing Derivatives of the Natural Amino Acid L-Tyrosine,” Mar. 31, 1994), RE37,160 E (“Synthesis of Tyrosine-Derived Diphenol Monomers,” May 1, 2001), and RE37,795 E (“Synthesis of Tyrosine-Derived Diphenol Monomers,” Jul. 16, 2002).

In one embodiment, polycarbonates and/or polyarylates are prepared using amino acid-derived diphenol compounds as starting materials. Useful amino acid-derived diphenol compounds include those described above with respect to polyiminocarbonates.

The amino acid-derived diphenol compounds can be used in any conventional polymerization process using diphenol monomers, including those processes that synthesize polymers traditionally considered hydrolytically stable and non-biodegradable. Accordingly, the amino acid-derived diphenol compounds of this embodiment can be used not only in the preparation of amino acid-derived polycarbonates and polyiminocarbonates, but they can also be used in the preparation of amino acid-derived polythiocarbonates, and polyethers as well. The amino acid-derived polymers demonstrate hydrolytic instability without sacrificing thermal stability or mechanical properties compared to their counterpart polymers not derived from amino acids.

Preferred amino acid-derived diphenol starting materials for the preparation of the amino acid-derived polycarbonates and/or polyarylates of this embodiment are monomers that are capable of being polymerized to form polyiminocarbonates with glass transition temperatures (“Tg's”) sufficiently low to permit thermal processing. The monomers according to this aspect are diphenol compounds that are amino acid derivatives of Formula 13: 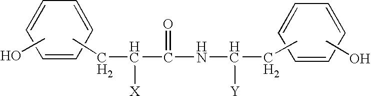
in which R₁ is an alkyl group containing up to 18 carbon atoms.

The particularly preferred amino acid-derived diphenol compound starting materials for the preparation of amino acid-derived polycarbonates and/or polyarylates according to this embodiment are derived from the naturally occurring amino acid L-tyrosine and its analog desaminotyrosine (Dat), which occurs naturally in plants. In this preferred group, the diphenol starting material can be regarded as a derivative of tyrosyl-tyrosine dipeptide from which the N-terminal amino group has been removed. The desaminotyrosyl-tyrosine compounds prepared by the methods are more properly referred to as desaminotyrosyl-tyrosine alkyl or alkylaryl esters. Preferred monomers of the group of desaminotyrosyl-tyrosine alkyl esters are the ethyl, butyl, hexyl, octyl, and benzyl esters.

The amino acid-derived diphenol starting materials are prepared by dicyclohexylcarbodiimide (DCC) mediated coupling reactions in THF following standard procedures of peptide chemistry such as described in Bodanszky, Practice of Peptide Synthesis (Springer-Verlag, NY, 1984) at page 145. The diphenols are recrystallized twice, first from 50% acetic acid in water and then from a 20:20:1 ratio of ethyl acetate, hexane, and methanol. Desaminotyrosyl-tyrosine hexyl ester (DTH) is prepared by DCC mediated coupling in THF of desaminotyrosine and tyrosine hexyl ester. The crude alkyl ester is obtained as an oil and purified by flash chromatography on silica gel using a 70:30 ratio of chloroform and ethyl acetate as the mobile phase. Crystallization of the pure product is accelerated by crystal seeding. Alkyl esters of tyrosine of up to eight carbon atoms in length are prepared according to the procedure disclosed in J. P. Greenstein and M. Winitz, Chemistry of the Amino Acids (John Wiley & Sons, NY 1961) at page 929, particularly Illustrative Procedure 10-48. Alkyl esters of tyrosine greater than eight carbon atoms in length are prepared according to the procedure disclosed in Overell, U.S. Pat. No. 4,428,932, particularly according to the procedure described by the examples.

Suitable polycarbonates and polyarylates can be prepared by conventional methods for polymerizing diphenols. This involves the reaction of the amino acid-derived diphenol compounds (as described herein) with phosgene or phosgene precursors (such as diphosgene or triphosgene, for example) in the presence of a catalyst. The amino acid-derived polycarbonates and/or polyarylates can be prepared by any of the processes known in the art for polymerization of such polymers, such as by interfacial polycondensation, polycondensation in a homogeneous phase, or by transesterification. Suitable processes, associated catalysts, and solvents are known in the art (see, for example, Schnell, Chemistry and Physics of Polycarbonates, Interscience, NY 1964).

Preferred amino acid-derived polycarbonates, formed by using the amino acid-derived diphenol compound starting materials described herein, thus include one or more recurring structural units represented by Formula 14: 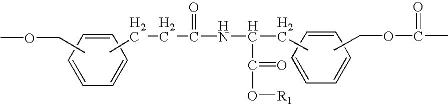
in which R₁ is an alkyl group up to 18 carbon atoms in length and preferably is a hexyl group. Amino acid-derived polycarbonates according to this aspect of the invention have intrinsic viscosities above about 0.5 dL/g, or in the range of 1.0 to 2.0 dL/g (chloroform, 30° C.) corresponding to weight-average molecular weights typically about 50,000 daltons and higher, or 100,000 daltons and higher.

Polycarbonates and polyiminocarbonates synthesized according to these aspects of the invention display varying degrees of hydrolytic instability. For example, poly(DTH-carbonate) is strong and tough, slowly degrading, and thermally stable. Poly(DTH-iminocarbonate) is very strong and brittle, fast degrading, with limited thermal stability.

In some embodiments, polymer blends of polycarbonates and polyiminocarbonates can be utilized to form the biodegradable polymeric material. According to these aspects, each polymer component of the blend is preferably derived from the same monomeric starting material. The two polymer components form highly compatible blends because they are derived from the same monomeric starting material. The blends can contain any ratio of polycarbonate and polyiminocarbonate. Preferred levels of each respective polymer in the blends will depend upon the requirements of the particular end use application, for example.

According to these embodiments, the blend components are completely miscible in all proportions and form macroscopically homogeneous blends from which clear, transparent films can be obtained. The tensile strength of the blends does not vary significantly with polyiminocarbonate content, although the ductility and hydrolytic stability of the blends decrease as the polyiminocarbonate content increases.

In still further embodiments, a rapidly-degrading poly(DTH-iminocarbonate) can be coated with a poly(DTH-carbonate) layer to obtain a strong and tough article that slowly degrades at first, but once degradation is initiated, rapidly disintegrates.

In still further embodiments, amino acid-derived diphenols can be copolymerized with dicarboxylic acids by way of a carbodiimide-mediated direct polycondensation to form nontoxic bioerodible polyarylates useful as biodegradable polymeric materials. The polyarylates according to these embodiments degrade by hydrolytic chain cleavage under physiological conditions. The polyarylates according to these embodiments employ diphenol compounds derived from dimers of L-tyrosine as a starting material.

L-tyrosine derived diphenol compounds suitable for the polymerization of polyarylates have the Formula 15: 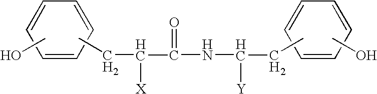
wherein X is selected from —H, —NHL₁, —NL₁L₂, or pendent groups having the Formula 16: 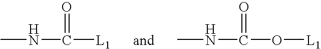
Y is a hydrogen or a pendent group having the Formula 17: 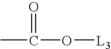
wherein L₁, L₂, and L₃ are independently selected from straight or branched alkyl and alkylaryl groups having up to 18 carbon atoms.

Among the preferred L-tyrosine derivatives of Formula 15 are derivatives in which X is hydrogen. These preferred compounds are tyrosine dipeptide analogs known as desaminotyrosyl-tyrosine and Y is: 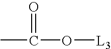
wherein L₃ is an alkyl or alkylaryl group containing up to 18 carbon atoms. In this preferred group, the diphenol starting material is properly referred to as a desaminotyrosyl-tyrosine alkyl or alkylaryl ester. Preferred desaminotyrosyl-tyrosine esters are the ethyl, butyl, hexyl, octyl, and benzyl esters. A particularly preferred ester is the hexyl ester, referred to as DTH.

The preparation of the diphenol starting materials can be depicted by the following scheme:

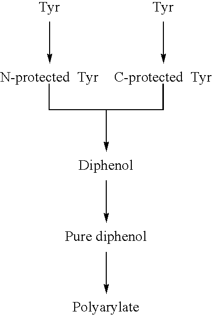

C-terminus protected alkyl and alkylaryl esters of tyrosine containing up to 8 carbon atoms are prepared according to standard procedures (see J. P. Greenstein and M. Winitz, supra). C-terminus protected alkyl and alkylaryl esters of tyrosine containing more than 8 carbon atoms are prepared according to procedures such as those described in Overell, supra.

N-terminus protected tyrosines are prepared following standard procedures of peptide chemistry such as disclosed in Bodanszky, supra. The protection of either terminus is omitted if X or Y of Formula 15 is hydrogen.

The crude tyrosine derivatives are sometimes obtained as oils and can be purified by simple recrystallization. Crystallization of the pure product can be accelerated by crystal seeding.

The diphenols are prepared by carbodiimide mediated coupling reactions in the presence of hydroxybenzotriazide following standard procedures of peptide chemistry such as disclosed in Bodanszky, supra, at page 145. The crude diphenols can be recrystallized twice, first from 50% acetic acid and water and then from a 20:20:1 ratio of ethyl acetate, hexane and methanol, or, alternatively, flash chromatography on silica gel is used, employing a 100:2 mixture of methylene chloride:methanol as the mobile phase. DTH is prepared by the carbodiimide mediated coupling of desaminotyrosine and tyrosine hexyl ester in the presence of hydroxybenzotriazole.

The diphenol compounds are then reacted with aliphatic or aromatic dicarboxylic acids in a carbodiimide-mediated direct polyesterification using 4-(dimethylamino)pyridinium-p-toluene sulfonate (“DPTS”) as a catalyst to form aliphatic or aromatic polyarylates. Dicarboxylic acids suitable for the polymerization of polyarylates have the structure of Formula 18: 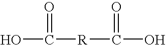
in which, for the aliphatic polyarylates, R is selected from saturated and unsaturated, substituted and unsubstituted alkylene or alkylarylene groups containing up to 18 carbon atoms, and preferably from 2 to 12 carbon atoms. For the aromatic polyarylates, R is selected from arylene groups containing up to 18 carbon atoms and preferably from 6 to 12 carbon atoms.

R is preferably selected so that the dicarboxylic acids employed as starting materials are either important naturally-occurring metabolites or highly biocompatible compounds. Preferred aliphatic dicarboxylic acid starting materials therefore include the intermediate dicarboxylic acids of the cellular respiration pathway known as the Krebs Cycle. These dicarboxylic acids include alpha-ketoglutaric acid, succinic acid, fumaric acid, maleic acid, and oxaloacetic acid, for which R is —CH₂—CH₂—C(═O)—, —CH₂—CH₂—, —CH═CH—, —CH₂—CH(OH)—, and —CH₂—C(═O)—, respectively.

Among the aliphatic dicarboxylic acids suitable for use in the invention are compounds in which R represents (—CH₂—)_{z}, wherein Z is an integer between zero and 12, inclusive. Thus an exemplary naturally occurring aliphatic dicarboxylic acid starting material is adipic acid (R=(—CH₂—)₄), found in beet juice. Other preferred biocompatible aliphatic dicarboxylic acids include sebacic acid (R=(—CH₂—)₈), oxalic acid (no R group), malonic acid (R=(—CH₂—)), glutaric acid (R=(—CH₂—)₃, pimelic acid (R=(—CH₂—)₅), suberic acid (R=(—CH₂—)₆), and azelaic acid (R=(—CH₂—)₇).

Preferred aromatic dicarboxylic acids suitable for use in these embodiments are base-sensitive, and the polyarylates are prepared by direct polyesterification, rather than by diacid chloride techniques. Polyesterification condensing agents and reaction conditions should be chosen that are compatible with the base-sensitive diphenol starting materials. Thus, the polyarylates can be prepared by the process disclosed by Ogata et al., Polym. J., 13(10), 989-91 (1981) and Yasuda et al., J.Poly.Sci. Polym. Chem. Ed., 21, 2609-16(1983), using triphenylphosphine as the condensing agent, the process of Tanaka et al., Polym. J., 14(8), 643-8 (1982) using picryl chloride as the condensing agent, or by the process of Higashi et al., J. Poly. Sci.:Polym.Chem.Ed., 24, 589-94 (1986) using phosphorus oxychloride as the condensing agent with lithium chloride monohydrate as a catalyst.

Other suitable synthesis processes include methods disclosed by Higashi et al. J. Polym. Sci: Polym. Chem. Ed., 21, 3233-9(1983) using arylsulfonyl chloride as the condensing agent, by the process of Higashi et al., J. Poly. Sci.: Polym. Chem. Ed., 21, 3241-7 (1983) using diphenyl chlorophosphate as the condensing agent, by the process of Higashi et al., J. Poly. Sci.: Polym. Chem. Ed., 24, 97-102 (1986) using thionyl chloride with pyridine as the condensing agent, or the process of Elias et al., Makromol. Chem., 182, 681-6 (1981) using thionyl chloride with triethylamine. A preferred polyesterification procedure is the method disclosed by Moore et al., Macromol., 23, 65-70 (1990) utilizing carbodiimide coupling reagents as the condensing agents with the specially designed catalyst DPTA.

One preferred polyesterification technique modifies the method of Moore et al. to utilize an excess of the carbodiimide coupling reagent. This produces aliphatic polyarylates having molecular weights greater than those obtained by Moore et al. Essentially any carbodiimide commonly used as a coupling reagent in peptide chemistry can be used as a condensing agent in this polyesterification process. Such carbodiimides are well known and include, for example, dicyclohexylcarbodiimide, diisopropylcarbodiimide, 1-(3-dimethylaminopropyl)-3-ethyl carbodiimide hydrochloride, N-cyclohexyl-N′-(2′-morpholinoethyl)-carbodiimide-metho-p-toluene sulfonate, N-benzyl-N′-3′-dimethylaminopropyl-carbodiimide hydrochloride, 1-ethyl-3-(3-dimethylaminopropyl)carbodiimide methiodide, N-ethylcarbodiimide hydrochloride, and the like. Preferred carbodiimides include dicyclohexyl carbodiimide and diisopropylcarbodiimide.

A reaction mixture is formed by contacting equimolar quantities of the diphenol and the dicarboxylic acid in a solvent for the diphenol and the dicarboxylic acid. Suitable solvents include methylene chloride, tetrahydrofuran, dimethylformamide, chloroform, carbon tetrachloride, and N-methylpyrrolidinone. It is not necessary to bring all reagents into complete solution prior to initiating the polyesterification reaction, although the polymerization of slightly soluble monomers such as desaminotyrosyltyrosine ethyl ester and succinic acid will yield higher molecular weight polymers when the amount of solvent is increased. The reaction mixture can also be heated gently to aid in the partial dissolution of the reactants.

In this particular method, the reaction mixture will also contain an excess of the carbodiimide coupling reagent. When carbodiimides are used in peptide synthesis as disclosed by Bodanszky (supra), between 0.5 to 1.0 molar equivalents of carbodiimide reagent is used for each mole of carboxylic acid group present. In the invention, greater than 1.0 molar equivalents of carbodiimide per mole of carboxylic acid group present are used (an excess of carbodiimide).

The polymer molecular weight significantly increases as the amount of coupling reagent used is increased. The degree of molecular weight increase only begins to level off around four molar equivalents of carbodiimide per mole of carboxylic acid group. Increasing the amount of coupling reagent beyond four equivalents of carbodiimide has no further beneficial effect. While quantities of carbodiimide greater than four equivalents are not detrimental to the polyesterification reaction, such quantities are not cost-effective and are thus not preferred.

The carbodiimide-mediated direct polyesterification is performed in the presence of the catalyst DPTS. DPTS is prepared in accordance with the procedure of Moore et al., Macromol., 23 (1), 65-70 (1990). The amount of DPTS is not critical because the material is a true catalyst that is regenerated. The catalytically effective quantity is generally in the range of about 0.1 to about 2.0 molar equivalents per mole of carboxylic acid group, and preferably about 0.5 equivalents per mole of carboxylic acid group.

The reaction proceeds at room temperature (about 20° to 30° C.). The reaction mixture can be heated slightly (<60° C.) prior to carbodiimide addition to partially solubilize less soluble monomers. However, the polymerization reaction itself is preferably conducted at temperatures in the range of 20° to 30° C. Within this temperature range, the reaction can be continued, with stirring, for at least 12 hours, and preferably for 1 to 4 days. The polymer is recovered by quenching the reaction mixture in methanol, from which the polyarylate usually precipitates while the residual reagents remain in solution. The precipitate can be separated by mechanical separations (such as filtration) and purified (such as by solvent washing).

In one preferred procedure, equimolar amounts of pure, dried tyrosine-derived phenol and dicarboxylic acid are weighed precisely (+/−0.0001 g), placed in a round-bottomed flask, and pre-dried at 130° C. A suitable magnetic stir bar is placed into the flask. Then 0.4 equivalents of DPTS are added. The flask is fitted with a septum and flushed with nitrogen or argon to remove traces of moisture from the reaction mixture. Next, a quantity of HPLC grade methylene chloride is added via a syringe and the reaction mixture is stirred vigorously to suspend the reactants. The amount of methylene chloride used will depend upon the solubility of the diphenol, or the dicarboxylic acid, or both monomers. At this stage, the reaction mixture can be slightly heated to partially dissolve the monomers. While it is not essential that the monomers be completely dissolved, the quantity of solvent should be sufficient to dissolve the polymer as it forms and thus slowly bring the monomers into solution.

Next, 4.0 equivalents of diisopropylcarbodiimide are added to the reaction mixture via a syringe. After about 10 minutes, the reaction mixture becomes clear, followed by the formation of a cloudy precipitate of diisopropylurea. After stirring at a temperature in the range of 20° to 30° C. for one to four days, the reaction is terminated by pouring the reaction mixture slowly and with vigorous stirring into ten volumes of methanol. The polymer precipitates while the residual reagents remain dissolved in methanol, resulting in the formation of the clear supernatant.

The polymeric product is retrieved by filtration and washed with copious amounts of methanol to remove any impurities. If desired, the polymeric products can be further purified by dissolving in methylene chloride (10% or 20% w/w) and reprecipitating in methanol. The polymeric product is then dried to constant weight under high vacuum.

The resulting polyarylates according to these particular embodiments include one or more recurring structural units represented by Formula 19: 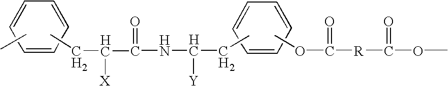
wherein X is selected from —H, —NHL₁, —NL₁L₂, or pendent groups having the Formula 20; 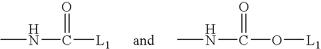
wherein Y is a hydrogen or a pendent group having the structure represented in Formula 21: 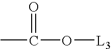
wherein L₁, L₂, and L₃ are independently selected from straight or branched alkyl and alkylaryl groups having up to 18 carbon atoms; and
R is selected from saturated and unsaturated, substituted and unsubstituted alkylene, arylene, and alkylarylene groups containing up to 18 carbon atoms.

In preferred embodiments, the recurring structural units of polyarylates are represented by Formula 19, with X being hydrogen and Y being the ester shown in Formula 21, with L₃ being an alkyl or alkylaryl group containing up to 18 carbon atoms and preferably being an ethyl, butyl, hexyl, octyl, or benzyl group. R is a saturated or unsaturated, substituted or unsubstituted, alkylene, arylene, or alkylarylene group containing up to 18 carbon atoms and is preferably a saturated or unsaturated, substituted of unsubstituted alkylene group containing 2 to 12 carbon atoms.

Preferred polyarylates have weight-average molecular weights above 50,000 daltons, and preferably above 100,000 daltons.

In alternative embodiments, the diphenol compounds can have the structure of Formula 22: 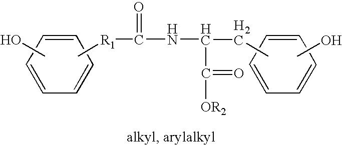
wherein R₁ is —CH═CH—, or (—CH₂—)ₙ, in which n is zero (i.e., where R₁ is a covalent bond) or an integer from one to eight; and R₂ is selected from straight and branched alkyl and alkylaryl groups containing up to 18 carbon atoms.

These diphenol compounds can be synthesized according to modified methods that include the step of coupling a hydroxyphenyl carboxylic acid having the structure of Formula 23: 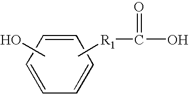
wherein R₁ is as described above with respect to Formula 22, with a tyrosine ester having the structure of Formula 24: 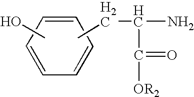
wherein R₂ is as described above with respect to Formula 22, in a water-miscible organic reaction solvent containing a carbodiimide capable of forming a water-soluble urea by-product thereby forming a diphenol reaction product. Upon completion of the coupling reaction, the reaction mixture is combined with an amount of water effective to precipitate the diphenol as a water-immiscible organic phase. In this way, two phases are formed, a water-immiscible organic phase containing the bulk of the diphenol reaction product, and an aqueous phase containing the bulk of the water-soluble urea and unreacted starting materials.

The tyrosine ester of Formula 24 is a C-terminus protected tyrosine. Such C-terminus protection is obtained by the formation of alkyl and alkylaryl esters of the C-terminus. C-terminus protecting alkyl and alkylaryl esters of tyrosine containing up to eight carbon atoms can be prepared according to known procedures (see, for example, J. P. Greenstein and M. Winitz, Chemistry of the Amino Acids, John Wiley & Sons, New York 1961, p. 927-929). C-terminus protecting alkyl and alkylaryl esters of tyrosine containing more than eight carbon atoms can be prepared by known techniques (see, for example, Overell, U.S. Pat. No. 4,428,932).

If the tyrosine alkyl or alkylaryl esters are initially obtained in their salt form, the salts can be removed by simple treatment with aqueous base. The diphenol compounds can then be prepared by coupling reactions mediated by carbodiimides capable of forming water-soluble urea by-products in water-miscible organic reaction solvent in which the carbodiimide, the hydroxyphenyl carboxylic acid, and the tyrosine ester are soluble. Examples of carbodiimides suitable for use with these embodiments that form water-soluble urea by-products include 1-ethyl-3-(3-dimethylamino-propyl)carbodiimide hydrochloride (EDCL.HCL), 1-alkyl-3-(3-dimethylaminopropyl)-3-(2-morpholinyl-(4)-ethyl) carbodiimide, 1-cyclohexyl-3-(4-diethylaminocyclohexyl) carbodiimide, 1-cyclohexyl-3-(B-diethylaminoethyl) carbodiimide, 1,3-di-(4-diethylaminocyclohexyl) carbodiimide, 1-alkyl-3-(3-morpholinyl-(4)-propyl)carbodiimide (alkyl-methyl, ethyl), 1-benzyl-3-(-dimethylamino-(N)propyl) carbodiimide, 1-ethyl-3-(4-azonia-4,4-di-methylpentyl)carbodiimide, in each case, as the free base or salt (HCL, methiodide, metho-p-toluenesulfonate). A preferred carbodiimide is EDCL.HCL.

Examples of suitable water-miscible organic solvents include tetrahydrofuran (THF), dioxane, dimethoxyethane, acetone, N-methylpyrrolidinone, and acetonitrile. A preferred solvent is THF.

The methods for forming suitable polyarylates otherwise essentially follow standard procedures of peptide chemistry as discussed herein. Typically, equimolar quantities of the hydroxyphenyl carboxylic acid and tyrosine ester are placed in a reaction vessel equipped with stirring means. The vessel is sealed and blanketed with an inert gas such as nitrogen, and a sufficient quantity of solvent is added to dissolve the hydroxyphenyl carboxylic acid and tyrosine ester, as well as the carbodiimide to be added. This quantity of solvent can be readily determined by one of skill in the art with reference to the teachings herein, and without undue experimentation.

The reaction mixture is then cooled to a temperature of about 0° C. prior to addition of the carbodiimide, which is then added to the reaction mixture while maintaining the inert blanket. The reaction mixture is stirred at the reduced temperature for at least one hour and allowed to gradually return to room temperature with stirring for at least one hour, and preferably about 19 hours.

The reaction mixture is then combined with an amount of water effective to precipitate the diphenol reaction product as a water-immiscible organic phase. At least 2 volumes of water are utilized relative to the reaction solvent, and preferably about 10 volumes of water are utilized.

Alternatively, the reaction solvent can be evaporated to leave a concentrated syrup-like residue. The residue is then washed with water to precipitate the diphenol reaction product as a water-immiscible organic phase, while the urea by-product is extracted into the aqueous phase.

The diphenol-containing water-immiscible organic phase is then separated from the aqueous phase, typically by addition of a water-immiscible organic solvent such as methylene chloride, chloroform, or ethyl acetate. The purpose of adding the water-immiscible solvent at this stage is to dilute the highly concentrated diphenol-containing residue and to facilitate the separation of the diphenol from the aqueous phase. The preferred solvent for preparation of desaminotyrosyl-tyrosine ethyl ester (DTE) is ethyl acetate, and for preparation of all other monomers is methylene chloride. At least 2 volumes of the extraction solvent should be utilized relative to the original quantity of reaction solvent employed.

At this stage, the organic phase can be dried over MgSO₄ or Na₂SO₄, filtered, and concentrated to an oil that can be placed under hexane to precipitate highly pure crystals of the diphenol reaction product. Preferably, the water-immiscible organic phase is backwashed with either or both aqueous acid and mild base extraction media to further purify the organic phase of water-soluble contaminants. Preferably, the organic phase is ultimately washed with multiple portions of both acid and mild base aqueous extraction media. For example, the organic phase can first be washed with multiple portions of 0.1M Na₂CO₃, followed by multiple portions of saturated NaCl, multiple portions of 0.1M citric acid or hydrochloric acid, and multiple portions of saturated NaCl. The volume of extraction media to be utilized for each portion is well known by those of ordinary skill in the art and should be slightly greater in volume than the organic phase.

The aqueous layers are preferably further backwashed with equal volumes of the organic phase solvent. The organic phases should then be combined, dried over MgSO₄, filtered, and concentrated to an oil, from which the diphenol reaction product can be recovered under hexane as described herein.

These methods can be utilized to synthesize diphenol compounds of Formula 15, as well as diphenol compounds of Formula 22 wherein R₁ is —CH═CH—, or (—CH₂—)ₙ, in which n is zero or one, or an integer from three to eight; and R₂ is selected from straight and branched alkyl and alkylaryl groups containing up to 18 carbon atoms.

The diphenol compounds are then polymerized to form tissue compatible biodegradable polymers. For example, the diphenol compounds can be polymerized to form polyiminocarbonates via one of the methods described elsewhere herein. According to one method, part of the diphenol is converted to the appropriate dicyanate, then equimolar quantities of the diphenol and the dicyanate are polymerized in the presence of a strong base catalyst such as a metal alkoxide or metal hydroxide. The resulting polyiminocarbonate will have the structure of Formula 25: 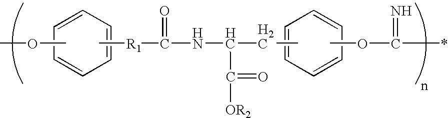
in which R₁ and R₂ are the same as described with respect to Formula 22, and n is an integer greater than 1.

The diphenol compounds can be utilized in interfacial polymerization methods described herein with respect to polyiminocarbonates to form polyiminocarbonates of Formula 25. The diphenol can be reacted with cyanogen bromide in an interfacial polymerization to form a polyiminocarbonate having the structure Formula 24. The diphenol compounds can also be reacted with phosgene to form polycarbonates as described herein. Polycarbonates prepared in accordance with these methods utilizing the diphenols of these embodiments have repeating structural units with the structure of Formula 25 described above.

The diphenols can also be reacted according to methods described herein to form polyarylates as described herein. The diphenol compounds are reacted with aliphatic or aromatic dicarboxylic acids in a carbodiimide mediated direct polyesterification using DPTS as a catalyst to form aliphatic or aromatic polyarylates. Dicarboxylic acids suitable for the polymerization of polyarylates have the structures of Formula 18 (described above), or Formula 26: 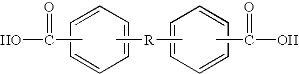
in which, for the aliphatic polyarylates, R is selected from alkylene or alkylarylene groups containing up to 18 carbon atoms, and preferably 2 to 12 carbon atoms. For the aromatic polyarylates, R is selected from arylene groups containing up to 18 carbon atoms and preferably 6 to 12 carbon atoms. The resulting aliphatic polyarylate has the structure of Formula 27, while the resulting aromatic polyarylate has the structure of Formula 28: 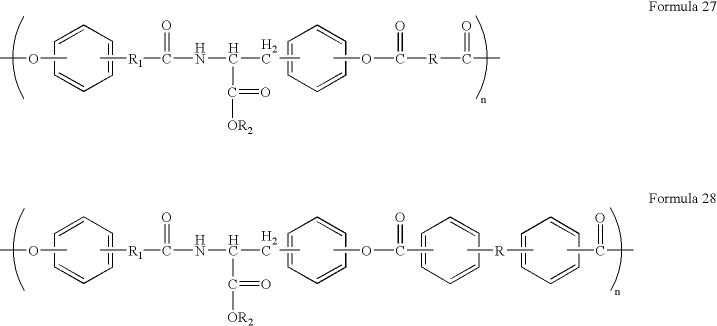
in which R is the same as described above with respect to Formula 18, and R₁ and R₂ are the same as described above with respect to Formula 22.

The diphenols according to these embodiments provide polyiminocarbonates having weight average molecular weights above about 60,000 daltons, up to about 200,000 daltons, and higher. The diphenols of these embodiments provide polyarylates having weight average molecular weights above about 50,000 daltons, and preferably above 100,000 daltons.

The amino acid-derived polyarylates and polycarbonates can be formed into drug delivery implants that degrade to release bioactive agents within a predictable controlled release time. Such controlled bioactive agent delivery systems can be prepared by incorporating the bioactive agents into the polymer chains as pendent side chains or by cross linking the pendent side chains to form a polymeric matrix into which the active agents are physically embedded or dispersed. Controlled bioactive agent delivery system implants can also be formed by physically admixing the polyarylates and/or polycarbonates with a bioactive agent.

Bioactive agents can be chemically incorporated into pendent side chains of polyarylates having repeating structural units according to Formula 19, wherein X is an unprotected amino group (N-terminus) or Y is an unprotected carboxylic acid group (C-terminus). The formation of polyarylates having de-protected N- or C-termini from the polyarylates of these embodiments can be achieved by the use of temporary protecting groups known in the art of peptide synthesis (see, for example, Bodanszky, supra).

A variety of bioactive agents having functional groups capable of being coupled to a free amino or carboxylic acid group can then be covalently incorporated into the deprotected monomeric units of the polyarylates by conventional coupling techniques. The resulting polyarylate has repeating structural units according to Formula 19 in which X is selected from
—NHL₁, —NL₁L₂; 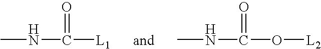
or Y is 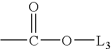
   wherein L₁, L₂, or L₃ is a bioactive agent.

L₁ or L₂ of X include bioactive agents that include a free carboxylic acid group through which the bioactive agent is covalently bonded. L₃ of Y includes bioactive agents that include a free amino or hydroxyl group, through which the bioactive agent is covalently bonded to Y.

Optionally, the bioactive agent is physically embedded or dispersed into the polymeric matrix or physically admixed with a polyarylate and/or polycarbonate. According to these embodiments, the bioactive agent can include any bioactive agent that is selected for controlled delivery over time.

In some aspects, the polymeric material can be prepared from dihydroxy monomers in which an α, β, or γ-hydroxy acid is first linked with an L-tyrosine alkyl ester or a structural derivative of L-tyrosine alkyl esters to form a dihydroxy monomer. These monomers can then be polymerized to form strictly alternating poly(amide carbonates), or they are copolymerized with selected diacids to form poly(amide esters), or they are reacted to form other useful polymers. Suitable monomers and resulting polymeric materials are described in U.S. Pat. No. 6,284,862 (“Monomers Derived from Hydroxy Acids and Polymers Prepared Therefrom,” Sep. 4, 2001), and PCT/US98/03127 (WO 98/36013, Published Aug. 20, 1998).

According to these aspects, aliphatic-aromatic dihydroxy monomers according to Formula 29 are utilized: 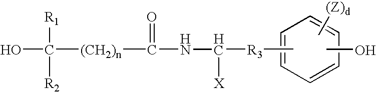
wherein R₁ and R₂ are each independently selected from H or straight or branched alkyl groups having up to 18 carbon atoms; R₃ is selected from the group —CH═CH—, and (—CH₂—)ₖ, wherein k is in the range of 0 to 6; each Z is an iodine or bromine atom; d and n are independently 0, 1, or 2; and X is hydrogen or a pendent group having the structure according to Formula 30: 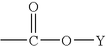
wherein Y is selected from straight or branched alkyl and alkylaryl groups having up to 18 carbon atoms. In preferred embodiments, n is zero, and R₁ and R₂ are preferably independently selected from hydrogen and methyl. Most preferably, n is zero and at least one of R₁ and R₂ is hydrogen, while the other, when not hydrogen, is methyl, resulting in the structures of glycolic acid and the various steroisomers of lactic acid, respectively. R₃ is preferably —CH₂—, so that the dihydroxy monomeric starting material is a derivative of L-tyrosine. X preferably has a structure according to Formula 30 in which Y is an ethyl, butyl, hexyl, octyl, or benzyl group. Y is preferably an ethyl group.

When at least one Z group is present, polymers prepared from the dihydroxy monomeric starting materials are radio-opaque. The iodinated and brominated dihydroxy monomers can also be employed as radio-opacifying, biocompatible non-toxic additives for other polymeric biomaterials, as described herein.

These monomers are similar to the desaminotyrosyl-tyrosine alkyl esters described above, with the important difference that the desaminotyrosyl-tyrosine unit has been replaced by aliphatic hydroxy acids. In particular, these dihydroxy monomers are water-soluble, as compared to the sparingly soluble desaminotyrosyl-tyrosine alkyl esters previously described. Further, these monomers can be utilized in the same fashion as the desaminotyrosyl-tyrosine alkyl esters disclosed herein. In particular, the monomers can be used to prepare polycarbonates, polyiminocarbonates, polyurethanes, poly(ester amides), and polyethers. Also, the monomers can be used to prepare aliphatic-aromatic poly(amide carbonates), prepared by processes described herein, as well as aliphatic-aromatic poly(amide esters) prepared by processes described herein.

Aliphatic-aromatic poly(amide carbonates) can be prepared having the repeating structural units of Formula 31:

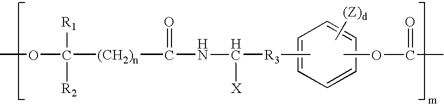

Aliphatic-aromatic poly(amide esters) can be prepared having the repeating structural units of Formula 32:

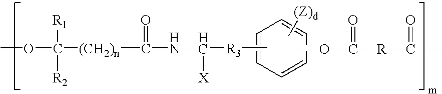

In Formulae 31 and 32, R₁, R₂, R₃, X, Z, d and n are as defined in Formulae 29 and 30. In addition, Y of X can also be hydrogen. R is selected from saturated and unsaturated, substituted and unsubstituted alkyl, aryl, and alkylaryl groups containing up to 24 carbon atoms; and m is the number of repeat units in the average polymer chain and can be in the range of 2 to 1,000.

The poly(amide carbonates) and poly(amide esters) will degrade faster and will bioresorb faster than polycarbonates and polyarylates polymerized from desaminotyrosyltyrosine alkyl esters. The polymeric materials can thus be used as biomaterials in situations that require a faster degradation and resorption rate than other polymeric materials.

Generally speaking, these dihydroxy monomers derived from tyrosine can be used in any conventional polymerization process using diol or diphenol monomers, including polyesters, polycarbonates, polyiminocarbonates, polyarylates, polyurethanes, polyethers, and random block copolymers of the dihydroxy monomers with poly(alkylene oxide). Particularly preferred embodiments are poly(amide esters) and poly(amide carbonates) as described in detail below.

The dihydroxy monomers of Formula 29 can be prepared by reacting an alkyl or alkylaryl ester of L-tyrosine that may or may not be iodinated or brominated with a hydroxy acid having the structure of Formula 29a: 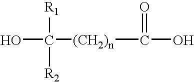
wherein R₁, R₂, and n are the same as described above with respect to Formula 29. The L-tyrosine ester is preferably an ethyl, butyl, hexyl, octyl, or benzyl ester. The ethyl ester is most preferred. For the dihydroxy acid of Formula 29a, when n is zero and R₁ and R₂ are hydrogen, the hydroxy acid is glycolic acid; and when n is zero, R₁ is hydrogen and R₂ is methyl, and the hydroxy acid is any of the stereoisomers of lactic acid. Glycolic acid is the most preferred dihydroxy compound starting material.

Preparation of alkyl and alkylaryl esters of tyrosine containing up to eight carbon atoms is discussed elsewhere herein. Preparation of alkyl and alkylaryl esters of tyrosine containing more than eight carbon atoms is discussed elsewhere herein. If the tyrosine alkyl or alkylaryl esters are initially obtained in their salt form, salts can be removed by a simple washing with aqueous base.

The dihydroxy compounds are then prepared by carbodiimide-mediated coupling reactions in the presence of hydroxybenzotriazide and utilizing carbodiimides according to the procedures described elsewhere herein. A preferred carbodiimide is 1-ethyl-3-(3-dimethylamino-propyl) carbodiimide hydrochloride (EDCL.HCL).

The crude dihydroxy compounds can be recrystallized twice, first from 50% acetic acid and water, and then from a 20:20:1 ratio of ethyl acetate, hexane and methanol. Alternatively, flash chromatography on silica gel is used, including a 100:2 mixture of methylene chloride methanol as the mobile base.

The dihydroxy compounds are then polymerized to form tissue compatible bioerodible polymeric materials. For example, the dihydroxy compounds can be polymerized to form polyiminocarbonates via one of the methods disclosed herein. The resulting polyiminocarbonate will have the structure of Formula 33: 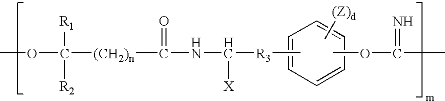
wherein R₁, R₂, R₃, X, Z, d, and are the same as described above for Formula 31, and m is the number of repeat units in the average polymer chain and can range from 2 to 1,000, inclusive.

The dihydroxy compounds can also be reacted with phosgene to form aliphatic-aromatic poly(amide carbonates) by the methods described herein. Aliphatic-aromatic poly(amide carbonates) prepared in accordance with these methods using the dihydroxy compounds have repeating structural units with the structure of Formula 31 in which R₁, R₂, R₃, X, Z, d, n, and m are the same as described with respect to Formula 31.

The dihydroxy compounds can also be reacted according to the methods described herein to form strictly alternating poly(amide esters). The dihydroxy compounds are reacted with aliphatic or aromatic dicarboxylic acids in a carbodiimide mediated direct polyesterification using 4-(dimethylamino) pyridinium-p-toluene sulfonate (DPTS) as a catalyst to form the aliphatic or aromatic poly(ester amides). Dicarboxylic acids suitable for the polymerization of poly(ester amides) have the structure of Formula 18: 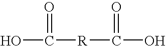
in which, for the aliphatic poly(ester amides), R is selected from saturated and unsaturated, substituted and unsubstituted alkylene groups containing up to 18 carbon atoms, and preferably from 2 to 12 carbon atoms that optionally may also include at least one nitrogen or oxygen atom. The resulting poly(amide ester) has the structure of Formula 32, in which R, R₁, R₂, R₃, X, Z, d, n, and m are the same as described above with respect to Formula 32.

R is preferably selected so that the dicarboxylic acids employed as the starting materials are either important naturally occurring metabolites or highly biocompatible compounds. Preferred aliphatic dicarboxylic acid starting materials therefore include the intermediate dicarboxylic acids of the cellular respiration pathway known as the Krebs Cycle. These dicarboxylic acids include alpha-ketoglutaric acid, succinic acid, fumaric acid, maleic acid, and oxalacetic acid. Other preferred biocompatible aliphatic dicarboxylic acids include sebacic acid, adipic acid, oxalic acid, malonic acid, glutaric acid, imelic acid, suberic acid, and azelaic acid. Among the preferred aromatic dicarboxylic acids are terephthalic acid, isophthalic acid, and bis(p-carboxylphenoxy) alkanes such as bis(p-carboxylphenoxy) alkanes, such as bis(p-carboxylphenoxy) propane.

The dihydroxy compounds can also be useful in the preparation of polyurethanes where various dihydroxy compounds are used as chain extenders by essentially conventional procedures. Random or block copolymers of the poly(amide carbonates) and poly(amide esters) with a poly(alkylene oxide) can be prepared according to procedures described later herein.

The dihydroxy compounds form poly(amide carbonates) having weight-average molecular weights above about 20,000 daltons, preferably about 80,000 daltons. The dihydroxy compounds provide poly(ester amides) having weight average molecular weights above about 20,000 daltons and preferably above about 80,000 daltons.

The polymeric materials include polymers having pendent free carboxylic acid groups. However, it is not possible to polymerize polymers having pendent free carboxylic acid groups from corresponding monomers with pendent free carboxylic acid groups without cross-reaction of the free carboxylic acid group with the co-monomer. Accordingly, polymers having pendent free carboxylic acid groups are prepared from homopolymers and copolymers of benzyl ester monomers having the structure of Formula 29 in which X has the structure of Formula 30 in which Y is a benzyl group.

The benzyl ester homopolymers and copolymers can be converted to corresponding free carboxylic acid homopolymers and copolymers through the selective removal of the benzyl groups by the palladium catalyzed hydrogenolysis methods described herein.

Preparation of the radio opaque bromine- and iodine-substituted polymeric materials as described herein can utilize commonly known techniques that will not be described further herein (See, for example, U.S. Pat. No. 6,602,497 “Strictly Alternating Poly(Alkylene Oxide Ether) Copolymers,” Aug. 5, 2003).

In one aspect, the polymeric materials formed utilizing the dihydroxy monomers can be utilized for tissue engineering, wherein new tissues are engineered by transplanting isolated cell populations on biomaterial scaffolds to create functional new tissue in vivo. For these applications, it is preferable to have relatively fast degradation and full resorption of the polymeric materials.

IV. Poly(Alkylene Oxide) Copolymers

In addition to the non-peptide polyamino acid polymers, polyiminocarbonates, and amino acid-derived polycarbonates and polyarylates described above, the biodegradable polymeric material can be composed of polycarbonates and/or polyarylates that include copolymers of poly(alkylene oxides) and amino acids or peptide sequences (hereafter, for discussion, referred to as “PAO copolymers”). These PAO copolymers will now be described in more detail.

According to these aspects of the invention, the biodegradable polymeric material is composed of copolymers of poly(alkylene oxides) and amino acids or peptide sequences, which amino acids or peptide sequences provide pendent functional groups at regular intervals within the polymer for bioactive agent attachment and/or crosslinking reactions. The resulting polymer is typically dominated by the desirable properties of the poly(alkylene oxide) (such as polyethylene glycol, or PEG), while the amino acid or peptide sequences provide biocompatible moieties having pendent functional groups for bioactive agent attachment and/or crosslinking.

Suitable biodegradable PAO copolymers are described, for example, in U.S. Pat. No. 5,219,564 (“Poly(alkylene oxide) Amino Acid Copolymers and Drug Carriers and Charged Copolymers Based Thereon,” Jun. 15, 1993) and U.S. Pat. No. 5,455,027 (“Poly(alkylene oxide) Amino Acid Copolymers and Drug Carriers and Charged Copolymers Based Thereon,” Oct. 3, 1995). According to these aspects, a polymer is provided in which a terminal amino group and an amino acid or peptide sequence are copolymerized with poly(alkylene oxides) having terminal hydroxyl groups by way of hydrolyzable ester linkages. The resulting copolymer includes multiple pendent functional groups at regular predetermined intervals.

The polymer contains one or more recurring structural units independently represented by the structure Formula 34:
-L-R₁-L-R₂—
wherein R₁ is a poly(alkylene oxide), L is —O— or —NH—, and R₂ is an amino acid or peptide sequence containing two carboxylic acid groups and at least one pendent amino group. In this aspect, the amino group is not involved in the polymerization process and is thus retained as a pendent group on the polymer that can be further derivatized, used for crosslinking, and/or used for the attachment of ligands. Preferably, R₂ is represented by Formula 35: 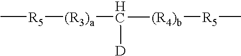
wherein R₃ and R₄ are independently selected from saturated and unsaturated, straight-chain and branched alkylene groups containing up to 6 carbon atoms and alkyl phenylene groups, the alkyl portions of which are covalently bonded to an amine and contain up to 6 carbon atoms. The values for a and b are independently zero or one. R₅ is independently Formula 36: 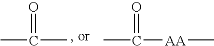
   wherein -AA- is an amino acid or peptide sequence, with the proviso that -AA- contains a free C-terminus (such that, when present, R₂ represents a peptide sequence of two or more amino acids). The polymers of formula 34 possess pendent functional groups at regular intervals within the polymer. D is a pendent functional group representing either —NHZ or —NH—X₁-Z. When D is —NHZ, Z is hydrogen, and the pendent functional groups are amino groups. The pendent amino groups can be further functionalized, in which case Z is selected from: 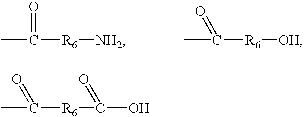
   Z can also be an N-terminus protecting group or a derivative of a bioactive agent covalently bonded to the pendent functional group by means of an amide bond in the case when in the underivatized bioactive agent a carboxylic acid group is present at the position of the amide bond in the derivative. N-terminus protecting groups are well known to those of ordinary skill in the art and will not be described in further detail herein. Preferred N-terminus protecting groups include benzyloxycarbonyl and tert-butoxycarbonyl groups.

When D is —NH—X₁-Z, Z is a bioactive agent covalently bonded to the pendent functional group by means of X₁. X₁ is a linkage selected from Formula 37: 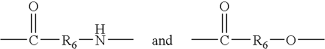
in the case when in the underivatized bioactive agent a carboxylic acid is present at the position linked to the pendent functional group by means of X₁; and 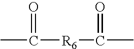
in the case when in the underivatized bioactive agent a primary or secondary amine or primary hydroxyl is present at the position linked to the pendent functional group by X₁. R₆ is selected from alkylene groups containing from two to six carbon atoms, arylene groups, alpha-, beta-, gamma-, and omega amino acids, and peptide sequences.

These polymers can, in some embodiments, be utilized for targeted delivery of bioactive agent. Z can be a derivative of a monoclonal antibody having oxidized carbohydrate moieties covalently bonded to the pendent amino group of the recurring structural unit by means of an amide bond in the case when in the underivatized oxidized monoclonal antibody a ketone or aldehyde is present at the position of the amide bond in the derivative. The polymer having pendent amino groups preferably contains both recurring structural units having oxidized monoclonal antibodies covalently bonded thereto at the pendent functional group and recurring structural units having a derivative of a bioactive agent covalently bonded thereto at the pendent functional group, with the monoclonal antibody and the bioactive agent preselected so that the monoclonal antibody targets cells for which it is specific for treatment by the bioactive compound with which is it conjugated.

Regarding the amino acid or peptide sequence represented by R₂ in Formula 34 and having a structure according to Formula 35, R₃ and R₄ are again preferably alkylene groups containing 1 to 4 carbon atoms, inclusive. When R₂ is an amino acid, R₅ is a carboxyl group. When R₂ is a peptide sequence, R₅ is 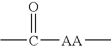
wherein the -AA- of R₅ is bonded to R₃ or R₄ by way of the carbonyl group of R₅.

The single amino acids and the two or more amino acids making up the peptide sequences are preferably alpha-amino acids, in which case a or b, or both, is zero, and -AA- represents one or more alpha-amino acids. More preferably, the single amino acids and the two or more amino acids making up the peptide sequences are natural amino acids, in which instance R₃ (when b is zero) or R₄ (when a is zero) is —CH₂— in the case of aspartic acid, —CH₂—CH₂— in the case of glutamic acid, and Formula 38: 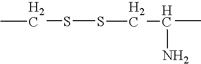
in the case of cystine. When present, -AA- would then represent one or more natural amino acids.

In some embodiments, the PAO copolymer can include both amide and ester recurring structural units, so that, with respect to Formula 34, L is —O— for some recurring structural units and —NH— for other recurring structural units. By varying the ratio of —O— and —NH—, the hydrolytic stability of the polymer can be tailored to suit the needs of the end use application.

The PAO copolymer of Formula 34 can be prepared according to the following solution polymerization process, in which the poly(alkylene oxide) and amino acid or peptide sequence are copolymerized by way of hydrolyzable ester linkages or a combination of hydrolyzable ester linkages and hydrolytically stable amide linkages. The process includes the steps of contacting a hydroxyl-terminated or amino-terminated poly(alkylene oxide) with an amino acid or a peptide sequence in an organic solvent in the presence of coupling reagent and an acylate catalyst, which amino acid or peptide sequence has at least two free carboxylic acid groups, with the proviso that when the poly(alkylene oxide) is hydroxyl-terminated, the amino acid or peptide sequence has protected N-terminals. The resulting copolymer of the poly(alkylene oxide) with the amino acid or peptide sequence is then recovered.

More specifically, the poly(alkylene oxide) is preferably dried by the azeotropic removal of water by distillation in toluene, followed by drying under vacuum. The solution polymerization is carried out in an organic solvent such as methylene chloride, chloroform, dichloroethane, and the like.

The poly(alkylene oxides) utilized in the reaction can include either hydroxyl terminals or amino terminals and are otherwise as described above. The poly(alkylene oxide) is dissolved in the solvent and stirred under argon. An equimolar quantity is then added of one or more of the amino acids or peptide sequences described herein. The reaction mixture can be heated slightly to dissolve the amino acid or peptide. The solution concentration of either compound is not critical. An excess quantity of a coupling reagent is also added to the reaction mixture, together with an excess quantity of an acylation catalyst.

Exemplary coupling reagents include, but are not limited to, carbodiimides such as ethyl dimethylaminopropyl carbodiimide (EDC), diisopropyl carbodiimide and 3-[2-morpholinyl-(4)-ethyl]carbodiimide, p-toluene sulfonate, 5-substituted isoxazolium salts, such as Woodward's Reagent K, and the like. Suitable coupling reagents and the quantities to employ are well known in the field of peptide synthesis and will not be discussed in further detail herein. Suitable acylation catalysts and the quantities to employ are also well known, and include, but are not limited to, dimethylaminopyridinium toluene sulfonate, hydroxybenzotriazole, imidazoles, triazole, dimethyl amino pyridine, and the like.

The reaction mixture is stirred at a temperature in the range of about 4° C. to about 40° C. and preferably at room temperature until completion of the reaction, typically within 24 hours.

The poly(alkylene oxide) reacts with the amino acid or peptide sequence to produce the copolymer of Formula 34. A urea precipitate is removed by filtration, and the polymer is then precipitated with cold ether, filtered, and dried under vacuum. The polymer can then be further purified by conventional methods, typically by reprecipitation from isopropanol.

In some embodiments, hydrogel membranes of polymer matrices formed from PAO copolymers can be formed, wherein the amino acids or peptide sequences include pendent acyl hydrazine groups. The copolymers are crosslinked by way of hydrolytically labile acyl semicarbazide linkages between a diisocyanate and the pendent acyl hydrazine groups of the polymer. Preferred hydrogel membranes of these embodiments, when incorporated with water, demonstrate high water content and high mechanical strength.

Other suitable polyarylate and polycarbonate PAO copolymers are described, for example, in U.S. Pat. No. 5,658,995 (“Copolymers of Tyrosine-Based Polycarbonate and Poly(alkylene oxide),” Aug. 19, 1997), U.S. Pat. No. 6,048,521 (“Copolymers of Tyrosine-Based Polycarbonate and Poly(alkylene oxide),” Apr. 11, 2000), and U.S. Pat. No. 6,319,492 (“Copolymers of Tyrosine-Based Polyarylates and Poly(Alkylene Oxides,” Nov. 20, 2001), as well as PCT Application No. U.S. Ser. No. 96/19098 (WO 97/19996, published Jun. 5, 1997). These polymeric materials are composed of polyarylate or polycarbonate random block copolymers that include tyrosine-derived diphenol monomers and poly(alkylene oxide).

In one such embodiment, the random block copolymer of a tyrosine-derived diphenol monomer and a poly(alkylene oxide) is provided having the structural Formula 39: 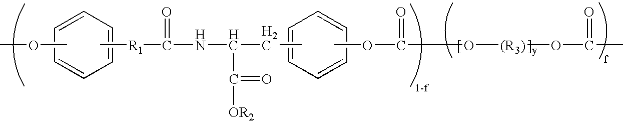
wherein R₁ is —CH═CH— or (—CH₂—)ⱼ, in which j is zero or an integer from one to eight;
R₂ is selected from straight and branched alkyl and alkylaryl groups containing up to 18 carbon atoms and optionally containing at least one ether linkage, and derivatives of biologically and pharmaceutically active compounds covalently bonded to the copolymer;
each R₃ is independently selected from alkylene groups containing 1 to 4 carbon atoms;
y is between 5 and about 3000; and
f is the percent molar fraction of alkylene oxide in the copolymer, and is in the range of about 1 to about 99 mole percent (f is 0.01 to 0.99). The defined units of tyrosine-derived diphenols and poly(alkylene oxide) do not imply the presence of defined blocks within the structure of Formula 39. The mole percent of alkylene oxide can vary over the entire range of about 5 to about 95 percent, with polymers having levels of alkylene oxide higher than 5 mole percent being resistant to cell attachment. Polymers with poly(alkylene oxide) levels higher than 70 mole percent are water soluble. Polymers with any level of alkylene oxide are useful for bioactive agent delivery, with water-soluble compositions being preferred for bioactive agent-targeting applications.

In preferred embodiments, the copolymers of these embodiments show inverse temperature transitions in aqueous solvents. Preferably, copolymers of these embodiments undergo continuous or discontinuous volume change upon changes in temperature, solvent composition, pH or ionic composition. The driving forces for the phase change can be attractive or repulsive electrostatic interactions, hydrogen bonding, or hydrophobic effects.

For nonionic synthetic polymers such as protein-based bioelastic materials, poly(N-isopropylacrylamide) and poly(ethylene glycol)-poly(propylene glycol) copolymers, as well as the copolymers of these embodiments, the driving force of phase transition is the combination of hydrogen bonding and hydrophobic effect. As the temperature increases, the gels of these polymers undergo a phase transition from a swollen to a collapsed state, while polymer solutions precipitate at certain temperatures or within certain temperature ranges. These polymers, including the copolymers of these embodiments, and especially those that undergo a phase transition at about 30° to 40° C. on heating can be used as polymeric materials according to these aspects of the polymeric material.

The introduction of poly(alkylene oxide) segments into the backbone of tyrosine-based polyarylates preferably provides softer, more hydrophilic polymers that exhibit increased rates of degradation compared to copolymers that lack the poly(alkylene oxide) segments. Preferably, the copolymers of these embodiments are formed by polymerization of a dicarboxylic acid with a tyrosine-derived diphenol and a poly(alkylene oxide), wherein an equimolar combined quantity of the diphenol and the poly(alkylene oxide) is reacted with a dicarboxylic acid in a molar ratio of the diphenol to the poly(alkylene oxide) between about 1:99 and about 99:1. According to these embodiments, the tyrosine-derived diphenol has the structure of formula 22: 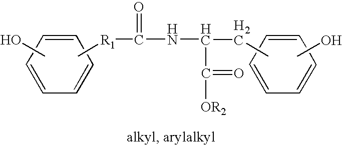
in which X is —O—, —NH—, or —N—; and R₁ and R₂ are the same as described above for formula 39. Preferably, R₁ is —CH₂—CH₂— and R₂ is preferably a straight chain ethyl, butyl, hexyl, or octyl group. R₂ can contain at least one ether linkage. When R₁ is —CH₂—CH₂—, the diphenol compound is referred to as a desaminotyrosyl-tyrosine alkyl ester. A preferred member of the group of desaminotyrosyl-tyrosine alkyl esters is the hexyl ester, DTH. The diphenols can be prepared as described elsewhere herein.

In some embodiments, R₂ can be a derivative of a bioactive agent covalently bonded to the copolymer or diphenol. R₂ is covalently bonded to the copolymer or diphenol by means of an amide bond when in the underivatized bioactive agent a primary or secondary amine is present at the position of the amide bond in the derivative. R₂ is covalently bonded to the copolymer or diphenol by means of an ester bond when in the underivatized bioactive agent a primary hydroxyl is present at the position of the ester bond in the derivative. The bioactive agent can also be derivatized at a ketone, aldehyde or carboxylic acid group with a linkage moiety that is covalently bonded to the copolymer or diphenol by means of an amide or ester bond.

Bioactive agents can also be physically blended with the random block copolymers using conventional techniques well-known to those of ordinary skill in the art.

The dicarboxylic acid has the structure of Formula 18: 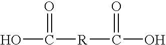
in which R is selected from saturated and unsaturated, substituted and unsubstituted alkylene, arylene, and alkylarylene groups containing up to 18 carbon atoms.

The poly(alkylene oxide) can be any commonly used alkylene oxide known in the art, and is preferably a poly(ethylene oxide), poly(propylene oxide), or poly(tetramethylene oxide). Poly(alkylene oxide) blocks containing ethylene oxide, propylene oxide, or tetramethylene oxide units in various combinations are also possible constituents. In preferred embodiments, the poly(alkylene oxide) has the structure of Formula 40:
(—O—R₃—)_{y}
in which each R₃ is independently selected from alkylene groups containing up to 4 carbon atoms and y is about 5 to about 3000. The poly(alkylene oxide) is preferably a poly(ethylene oxide) in which y of formula 40 is in the range of about 20 to about 200. More preferred embodiments are obtained when poly(ethylene oxide) blocks with a molecular weight in the range of about 1,000 to about 20,000 g/mol are used. For these preferred embodiments, in the structure of Formula 40, both R₃ groups are hydrogen and y has values in the range of about 22 to about 220, or in the range of about 22 to about 182.

The random block copolymers of these embodiments can be prepared by the conventional methods for polymerizing diphenols into polycarbonates described elsewhere herein. This involves the reaction of the desired ratio of tyrosine-derived diphenol and poly(alkylene oxide) with phosgene or phosgene precursors (such as diphosgene or triphosgene) in the presence of a catalyst. Thus, the copolymers of Formula 39 can be prepared by interfacial polycondensation, polycondensation in a homogeneous phase, or by transesterification. The suitable processes, associated catalysts, and solvents are well known in the art and will not be described further herein.

Preferred random block copolymers of Formula 39 have weight-average molecular weights above about 20,000 daltons, or above about 30,000 daltons. Preferred number-average molecular weights of the random block copolymers of Formula 39 are above about 10,000 daltons, or above about 20,000 daltons.

The tyrosine-derived diphenol compounds of Formula 22 and the poly(alkylene oxide) of Formula 39 can be reacted according to the methods described herein to form polyarylates. According to these embodiments, the diphenol compounds are reacted with the aliphatic or aromatic dicarboxylic acids of Formula 18 in a carbodiimide mediated direct polyesterification using 4-(dimethylamino)pyridinium-p-toluene sulfonate (DPTS) as a catalyst to form aliphatic or aromatic polyarylates. Random block copolymers with poly(alkylene oxide) can be formed by substituting poly(alkylene oxide) for the tyrosine derived diphenol compound in an amount effective to provide the desired ratio of diphenol to poly(alkylene oxide) in the random block copolymer.

With regard to any of the polymerization processes described herein, when the formation of the polymer involves a two-phase reaction medium (a biphasic polymerization reaction), processes such as those described in U.S. Pat. No. 6,359,102 (“Biphasic Polymerization Process,” Mar. 19, 2002) can be utilized. Generally, suitable processes involve control of pH and amine catalyst concentration to prepare polymeric products (such as polycarbonates, polyarylates, and the like). The biphasic polymerization process is particularly useful for polymerization of hydrolytically unstable diols, especially diphenols. According to these methods, polymerization is performed within a pH range of six to eight. Moreover, control of pH range and amount of catalysts provided can be used to control the polymer molecular weight. The biphasic polymerization processes are useful in polymerization processes wherein the monomers employed are hydrolytically unstable or hydrolytically stable.

Modifications of Biodegradable Polymeric Materials

In some preferred embodiments, the biodegradable polymeric material can be modified to provide enhanced performance of the implantable device. Exemplary modifications include incorporation of pendent carboxylic acid groups, formation of porous polymeric scaffold materials, and/or inclusion of additional polymers to control bioactive agent release. Each of these modifications will now be described.

I. Carboxylic Acid Groups

In some embodiments, pendent carboxylic acid groups can be incorporated within the polymer bulk for polycarbonates, polyarylates, and/or poly(alkylene oxide) block copolymers thereof, to further control the rate of polymer backbone degradation and resorption. The pendent carboxylic acid groups can be included on some or all of the monomeric subunits of the polymers. According to these embodiments, pendent carboxylic acid groups can be created on the polymer surface without concomitant backbone cleavage. These carboxylic acid groups can create chemically reactive attachment sites at the polymer surface. These particular embodiments will now be described in more detail.

Generally speaking, the polymeric materials are prepared by selectively removing the ester of pendent carboxylic acid groups from the polymer backbone. The resulting polymers contain pendent carboxylic acid groups on some or all of their monomeric repeating subunits. The pendent carboxylic acid groups can impart increased hydrophilicity to the polymers. Exemplary polymeric materials having pendent carboxylic acid groups and methods of preparing them are described in U.S. Pat. No. 6,120,491 (“Biodegradable, Anionic Polymers Derived from the Amino Acid L-Tyrosine,” Sep. 19, 2000).

Polymers according to these embodiments include homopolymers of a repeating unit having a pendent carboxylic acid group. Such homopolymers have the structure of Formula 41: 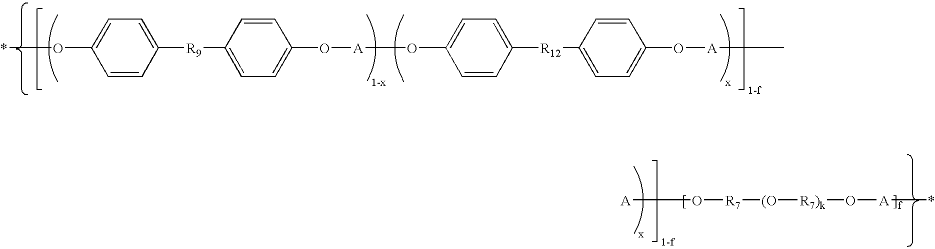
in which x and f are both zero and R₉ is the same as described herein with respect to Formula 42 (described below), with the proviso that it is limited to species having pendent carboxylic acid groups. The homopolymers are prepared by the hydrogenolysis of corresponding homopolymers having the structure of Formula 41 in which x and f are both zero and R₉ is the same as described herein with respect to Formula 42 (described below), with the proviso that it is limited to species having pendent benzyl carboxylate groups.

Polymers according to these embodiments also include copolymers having pendent carboxylic acid groups with the structure of Formula 41 in which f is zero, x is a number greater than zero but less than one, R₁₂ is the same as described herein with respect to Formula 46 and R₉ is the same as described herein with respect to Formula 42, with the proviso that it is limited to species with pendent carboxylic acid groups. In copolymers in accordance with these embodiments, x is preferably in the range of about 0.50 to about 0.90 and more preferably in the range of about 0.60 to about 0.80.

The polymers having pendent carboxylic acid groups are prepared by the hydrogenolysis of polymeric starting materials having corresponding pendent benzyl carboxylate groups. The benzyl carboxylate polymeric starting materials are polymerized from diphenol compounds having benzyl ester-protected pendent carboxylic acid groups, alone, or in combination with diphenol compounds having other ester-protected carboxylic ester groups. In particular, the benzyl carboxylate diphenols have the structure of Formula 42: 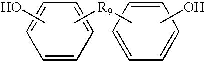
wherein R₉ is an alkylene, arylene, or alkylarylene group with up to 18 carbons with the proviso that this group contains as part of its structure a benzyl ester protected carboxylic acid group. R₉ can also contain non-carbon atoms such as nitrogen and oxygen. In some embodiments, R₉ can have a structure related to derivatives of the natural amino acid tyrosine, cinnamic acid, or 3-(4-hydroxypehnyl)propionic acid. In these cases, R₉ assumes the specific structures shown in formulae 43 and 44: 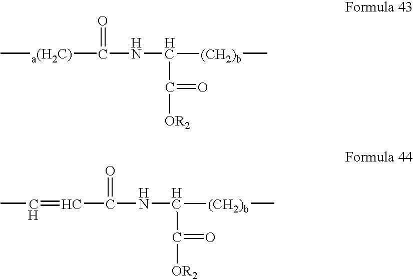
   The indicators a and b in Formulae 43 and 44 can be independently 0, 1, or 2. R₂ is hydrogen or a benzyl group.

The benzyl carboxylate diphenol starting materials preferably have the structure of Formula 42 in which R₉ has the structure of Formula 43 or 44 in which R₂ is a benzyl group. Among the preferred diphenols are compounds in which R₉ has the structure of Formula 43 in which a and b are independently one or two. Most preferably, a is two and b is one. These most preferred compounds are tyrosine dipeptide analogues known as desaminotyrosyl-tyrosine alkyl or alkylaryl esters. In this preferred group the diphenols can be regarded as derivatives of tyrosyl-tyrosine dipeptides from which the N-terminal amino group has been removed.

Diphenol compounds having the ester-protected carboxylic acid groups have the structure of Formula 45: 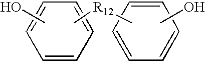
wherein R₁₂ is an alkylene, arylene, or alkylarylene group substituted with a carboxylic acid ester group, wherein the ester is selected from straight and branched alkyl and alkylaryl esters containing up to 18 carbon atoms, and ester derivatives of bioactive agents covalently bonded to the polymer, provided that the ester group is not a benzyl group or any other chemical moiety that can potentially be cleaved by hydrogenolysis. R₁₂ can also contain non-carbon atoms such as nitrogen and oxygen. In particular, R₁₂ can have a structure related to derivatives of the natural amino acid tyrosine, cinnamic acid, or 3-(4-hydroxyphenyl) propionic acid.

For derivatives of tyrosine, 3-(4-hydroxyphenyl)propionic acid and cinnamic acid, R₁₂ assumes the specific structures shown in Formulae 48 and 49: 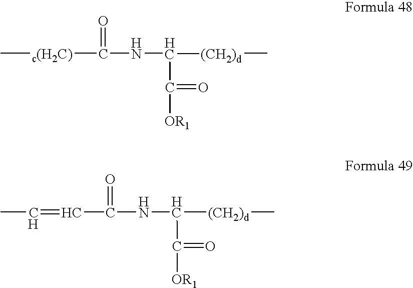
The indicators c and d can be independently 0, 1, or 2. R₁ is selected from straight and branched alkyl, alkylaryl, and aryl groups containing up to 18 carbon atoms, and ester derivatives of bioactive agents covalently bonded to the diphenol, provided that R₁ is not a benzyl group. More preferably, R₁₂ has the structure of Formula 47: 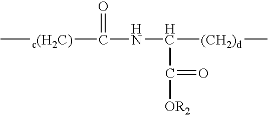
in which c and d are preferably independently one or two. Most preferably, c is two and d is one.

Methods for preparing the diphenol monomers are described herein. The preferred desaminotyrosyl-tyrosine esters according to these embodiments are the ethyl, butyl, hexyl, octyl, and benzyl esters. For purposes of this discussion, desaminotyrosyl-tyrosine ethyl ester is referred to as DTE, desaminotyrosyl-tyrosyl-tyrosine benzyl ester is referred to as DTBn, and the like. The desaminotyrosyl-tyrosine free acid is referred to as DT.

The polymers of these embodiments can be homopolymers with each monomeric subunit having a pendent carboxylic acid group prepared by the hydrogenolysis of corresponding benzyl carboxylate homopolymers. Copolymers of diphenol monomers having pendent carboxylic acid ester groups, and diphenol monomers having pendent carboxylic acid groups can also be incorporated into the basic backbone structure of the polymer by the hydrogenolysis of corresponding copolymers of benzyl ester monomers and monomers having pendent esters other than benzyl carboxylates.

Thus, for example, poly (DT carbonates) are prepared by the hydrogenolysis of poly(DTBn carbonates), poly (DT-DTE carbonate) copolymers are prepared by they hydrogenolysis of poly(DTBn-DTE carbonate) copolymers, and so forth. One can thus vary within polymers the molar ratios of the monomeric subunits having pendent alkyl and alkylaryl ester groups and the monomeric subunits having pendent carboxylic acid groups.

Benzyl esters of pendent polymer carboxylic acid groups can be selectively removed by palladium-catalyzed hydrogenolysis in N,N-dimethylformamide (DMF) or similar solvents such as N,N-dimethylacetamide (DMA) and N-methylpyrrolidone (NMP) to form pendent carboxylic acid groups. This results in selective removal of benzyl ester groups from biodegradable polycarbonates and polyarylates described herein. By varying the molar ratio of monomeric repeating subunits having pendent benzyl carboxylate groups to the monomeric repeating subunits having other alkyl or alkylaryl carboxylate groups within a polymer, the molar ratio of monomeric repeating subunits having pendent carboxylic acid groups within a polymer can be varied after completion of the selective removal of the benzyl carboxylate groups.

According to these embodiments, polymers include monomeric units defined in Formula 42 as follows:

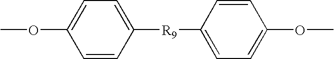

Formula 42 represents a diphenolic unit wherein R₉ is an alkylene, arylene, or alkylarylene group with up to 18 carbons with the proviso that this group contains as part of its structure a carboxylic acid group or the benzyl ester thereof. R₉ can also contain non-carbon atoms such as nitrogen and oxygen. In particular, R₉ can have a structure related to derivatives of the natural amino acid tyrosine, cinnamic acid, or 3-(4-hydroxypehnyl)propionic acid. In these cases, R₉ assumes the specific structures shown in Formulae 43 and 44: 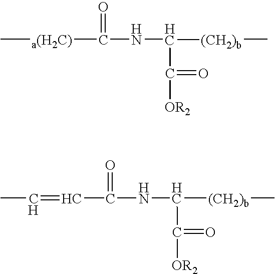
The indicators a and b in Formulae 43 and 44 can be independently 0, 1, or 2. R₂ is hydrogen or a benzyl group.

A second diphenolic subunit of the polymer is defined in Formula 46: 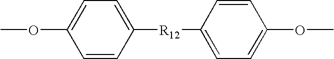
In this second diphenolic subunit, R₁₂ is as described herein with respect to Formula 45.

Some polymers of this invention can also contain blocks of poly(alkylene oxide) as defined in Formula 50:
—O—R₇—(O—R₇)ₖ
wherein R₇ is independently an alkylene group containing up to four carbon atoms, and k is 5 to 3,000.

A linking bond, designated as “A” is defined to be either of the structures indicated in Formula 51: 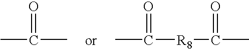
where R₈ is selected from saturated and unsaturated, substituted and unsubstituted alkylene, arylene, and alkylarylene groups containing up to 18 carbon atoms. Thus, polymers in accordance with these embodiments have the structure of Formula 41:

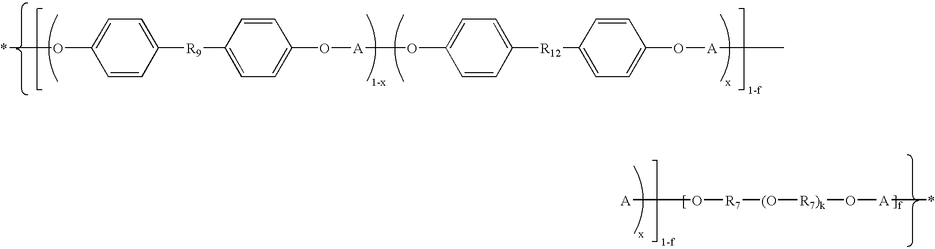

In Formula 41, x and f are the molar ratios of the various subunits. The references x and f can range from 0 to 0.99. It is understood that the presentation of Formula 41 is schematic and that the polymer structure presented by Formula 41 is a true random copolymer where the different subunits can occur in any random sequence throughout the polymer backbone. Formula 41 provides a general chemical description of polycarbonates when A is 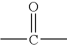
and of polyarylates when A is 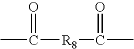
Furthermore, when x=0, the polymer contains only benzyl ester pendent chains which, after hydrogenolysis as described herein, will provide pendent carboxylic acid groups at each diphenolic repeat unit. If x is any fraction greater than 0 but smaller than 1, a copolymer is obtained that contains a defined ratio of benzyl ester and non-benzyl ester carrying pendent chains. After hydrogenolysis, a copolymer is obtained that contains a defined ratio of carboxylic acid groups as pendent chains.

If f=0, the polymers do not contain any poly(alkylene oxide) blocks. The frequency at which poly(alkylene oxide) blocks can be found within the polymer backbone increases as the value of f increases.

When A of Formula 41 is 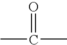
the polymers are polycarbonates. The polycarbonate homopolymer and copolymer starting materials having pendent benzyl carboxylate groups can be prepared by the methods described herein. Polycarbonate homopolymers and copolymers having pendent carboxylic acid groups, and the polycarbonates having pendent benzyl carboxylate groups from which they are prepared, have weight-average molecular weights in the range of about 20,000 to about 400,000 daltons, and preferably about 100,000 daltons.

When A of Formula 41 is 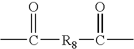
the polymers are polyarylates. The polyarylate homopolymer and copolymer starting materials having pendent benzyl carboxylate groups can be prepared by the methods described herein. Preferably, R₈ is not substituted with functional groups that would cross-react with the dicarboxylic acids. For the aliphatic polyarylates, R₈ is selected from saturated and unsaturated, substituted and unsubstituted alkylene groups containing up to 18 carbon atoms, preferably 4 to 12 carbon atoms. For aromatic polyarylates, R₈ is selected from arylene and alkylarylene groups containing up to 18 carbon atoms, preferably 8 to 14 carbon atoms. Again, R₈ is preferably not substituted with functional groups that would cross-react with the diphenols.

R₈ is preferably selected so that the dicarboxylic acids from which the polyarylate starting materials are polymerized are either important naturally-occurring metabolites or highly biocompatible compounds. Preferred aliphatic dicarboxylic acids therefore include the intermediate dicarboxylic acids of the Krebs Cycle and aliphatic and aromatic dicarboxylic acids described supra.

Polyarylate homopolymers and copolymers having pendent carboxylic acid groups, and the corresponding polyarylates having pendent benzyl carboxylate groups from which they are prepared, have weight average molecular weights in the range of about 20,000 to about 400,000 daltons, or about 100,000 daltons.

According to these embodiments, polycarbonates and polyarylates also include random block copolymers with a poly(alkylene oxide) having pendent carboxylic acid groups with the structure of Formula 41, wherein f is greater than zero but less than one, R₁₂ is the same as described herein with respect to Formula 46, k and R₇ are the same as described herein with respect to Formula 50 and R₉ is the same as described herein with respect to Formula 42, with the proviso that it is limited to species having pendent carboxylic acid groups. The value for x is less than one, but x may or may not be greater than zero.

The molar fraction of alkylene oxide in the block copolymer (f) ranges from about 0.01 to about 0.99. The block copolymers having pendent carboxylic acid groups are prepared by the hydrogenolysis of corresponding block copolymers having the structure of Formula 41, wherein x is greater than zero but less than one, R₁₂ is the same as described herein with respect to Formula 46, k and R₇ are the same as described herein with respect to Formula 50, and R₉ is the same as described herein with respect to Formula 42, with the proviso that it is limited to species having pendent benzyl carboxylate groups. Again, the value for x is less than one, but may or may not be greater than zero.

For preferred polymeric starting materials and the resulting free acid block copolymers, R₇ is ethylene, k is in the range of about 20 to about 200, and the molar fraction of alkylene oxide in the block copolymer (f) preferably is in the range of about 0.05 to about 0.75. R₇ can also represent two or more different alkylene groups within a polymer.

The block copolymers having pendent benzyl carboxylate groups can be prepared by the methods described herein. For block copolymers having either pendent carboxylic acid groups or pendent benzyl carboxylate groups in which x is greater than zero, the molar fraction of alkylene oxide and block copolymer (f) will remain in the range of about 0.01 to about 0.99.

The block copolymers having pendent carboxylic acid groups, and the block copolymers having pendent benzyl carboxylate groups from which they are prepared, have weight-average molecular weights in the range of about 20,000 to about 400,000 daltons, preferably about 100,000 daltons. The number-average molecular weights of the block copolymers are preferably above about 50,000 daltons.

For the copolymers having the structure of Formula 41 in which x is greater than zero, the pendent carboxylic acid ester group of R₁₂ can be an ester derivative of a bioactive agent covalently bonded to the polycarbonate or polyarylate copolymer. The covalent bond is by means of an amide bond when in the underivatized bioactive agent a primary or secondary amine is present at the position of the amide bond in the derivative. The covalent bond is by means of an ester bond when in the underivatized bioactive agent a primary hydroxyl is present at the position of the ester bond in the derivative. The bioactive agent can also be derivatized at a ketone, aldehyde or carboxylic acid group with a linkage moiety that is covalently bonded to the copolymer or diphenol by means of an amide or ester bond.

Detailed chemical procedures for the coupling of various bioactive agents to polymer bound free carboxylic acid groups have been described in, for example, Nathan et al., Bio. Cong. Chem., 4,54-62 (1993).

For purposes of these embodiments, bioactive agents can also be defined as including crosslinking moieties, such as molecules with double bonds (such as acrylic acid derivatives), which can be attached to the pendent carboxylic acid groups for crosslinking to increase the strength of the polymers. Bioactive agents, for purposes of these embodiments, are additionally defined as including cell attachment mediators, biologically active ligands, and the like.

The copolymers of these embodiments can contain about 1 to about 99 mole percent of monomeric subunits having pendent carboxylic acid groups. Their properties are strongly affected by the mole fraction of free carboxylic acid groups present. Copolymers that have less than 20 molar percent of monomeric repeating subunits with pendent carboxylic acid groups are processible by compression molding and extrusion. As a general rule, copolymers with less than 20 molar percent of monomeric repeating subunits with pendent carboxylic acid groups are not soluble in water.

For copolymers having more than 20 mole percent of monomeric subunits with pendent carboxylic acid groups, some thermal degradation has been observed during conventional compression molding and extrusion at elevated temperatures. Copolymers having more than 20 mole percent of monomeric subunits with pendent carboxylic acid groups tend to exhibit increased swelling (due to imbibition of water) during exposure to aqueous media and when more than about 50 mole percent of monomeric subunits carry free carboxylic acid groups, the copolymer tends to become water soluble and exhibits behavior similar to the behavior of the corresponding homopolymers (which dissolve in pH 7.4 phosphate buffer to the extent of about 2 mg/ml).

Irrespective of the amount of carboxylic acid groups, all copolymers of these embodiments are good film-forming materials. Copolymers having less than about 70 mole percent of monomeric subunits with pendent carboxylic acid groups can be processed into porous foams by salt leaching techniques (see, for example, Free et al., J. Biomed. Mater. Res., 27, 11-23(1993)), or by phase separation techniques (see Schugens et al., J. Biomed. Mater. Res. 30, 449-462 (1996)). Copolymers having more than about 70 mole percent of monomeric subunits with pendent carboxylic acid groups tend to be water soluble and must be processed into porous foams as described for the corresponding homopolymers.

The degradation/resorption of the polymers can be controlled by controlling the molar fraction of free carboxylic acid groups. Moreover, the composition of the polymers can also be used to influence interactions with cells. When the polycarbonates or polyarylates of these embodiments do not contain poly(alkylene oxide) (f=0 in Formula 41), they can be more adhesive growth substrates for cell cultures compared to ester-protected polymers. The negative charge from the free carboxylic acid groups present on the surface of the polymers can improve the attachment and growth of fibroblasts and can facilitate specific interactions with proteins, peptides, and cells. The polymer surfaces can also be modified by simple chemical protocols to attach specific peptides, in particular, the peptides containing variations of the “RGD” integrin binding sequence known to affect cellular attachment. Useful techniques for attachment of ligands to polymer-bound carboxylic acid groups are well-known in the art (see, for example, Nathan et al., Bioconj. Chem. 4,54-62 (1993)).

In some embodiments, incorporation of poly(alkylene oxide) blocks decreases the adhesiveness of the polymeric surfaces. Polymers for which f is greater than 5 mole percent according to Formula 41 are resistant to cell attachment and can be useful as non-thrombogenic coatings on surfaces in contact with blood. These polymers can also resist bacterial adhesion.

Generally speaking, the polymers having pendent carboxylic acid groups can be prepared by the palladium-catalyzed hydrogenolysis of corresponding polymers having pendent benzyl carboxylate groups. Essentially any palladium-based hydrogenolysis catalyst is suitable for use. Preferably, the palladium catalyst is palladium on barium sulfate, which catalyst is recoverable and reusable. Pure solvent (DMF, DMA, or NMP) is used as the reaction solvent.

More specifically, the polymers of these embodiments can be prepared by (1) preparing a reaction mixture of a polymer having the structure of Formula 41, in which R₉ has a pendent benzyl-protected carboxylic acid group, in an anhydrous reaction solvent consisting essentially of one or more solvents selected from DMF, DMA, and NMP, and (2) contacting the reaction mixture with a palladium catalyst in the presence of a hydrogen source so that the benzyl ester groups are selectively removed by hydrogenolysis.

A level of palladium on barium sulfate in the range of about 5 to about 10 percent by weight is preferred. Lower levels can extend reaction time and/or reduce yield, while higher levels can represent an unnecessary expense.

The polymer starting material having pendent benzyl carboxylate groups is preferably dissolved in DMF at a solution concentration (w/v %) in the range of about 5 to about 50 percent, or in the range of about 10 to about 20 percent.

The polymer is stirred until a clear solution is obtained. The palladium catalyst is then added, after which the hydrogen source is supplied to the reaction mixture.

The amount of palladium catalyst to be employed is that amount effective to catalyze the hydrogenolysis reaction. The absolute mass ratio of elemental palladium to the polymer is not as important as the surface activity of the elemental palladium. The amount of a catalyst preparation to be used will depend upon such factors as the specific catalytic activity of the preparation, and this can be readily determined by one of ordinary skill in the art without undue experimentation. For a preparation containing about 5 percent by weight of palladium on barium sulfate, about 15 to about 30 percent, preferably about 25 weight percent, of the preparation should be used relative to the polymeric starting material. If the catalyst preparation is being recycled, higher levels of the preparation should be used, because as the catalyst is reused, the palladium is slowly deactivated, and the amount used should be adjusted to maintain the stated catalytic activity. However, the increases in catalyst levels needed to adjust for the loss of catalytic activity can also be determined by one of ordinary skill in the art without undue experimentation.

Essentially any hydrogen source for palladium-catalyzed hydrogenolysis is suitable for use. For example, the reaction mixture can be supplied with a hydrogen gas blanket. Alternatively, a transfer hydrogenolysis reaction can be used. Preferred methods according to these embodiments use 1,4-cyclohexadiene, a transfer hydrogenolysis reagent, in combination with hydrogen gas as a hydrogen source. If desired, the reaction can be performed at high pressure in a PARR hydrogenolysis apparatus. At high-pressure conditions, the addition of 1,4-cyclohexadiene is not required to ensure removal of all benzyl ester groups from the polymers. Irrespective of the exact mode of conducting the reaction, it is important to maintain strictly anhydrous conditions.

When the transfer hydrogenolysis reagent is employed as a hydrogen source, a stoichiometric excess relative to the polymeric starting materials is preferably employed. With 1,4-cyclohexadiene, this represents an excess up to about 50 weight percent, and preferably about a 10 weight percent excess, relative to the polymeric starting material.

The progress of the reaction can be measured by monitoring the removal of the benzyl ester from the polymeric starting material in reaction aliquots by NMR spectroscopy. When the reaction has come to completion (typically about 24 to 48 hours), the polymer is isolated by filtering off the solid palladium catalyst and the filtrate is added into water to precipitate the polymer. The polymer can then be purified by suitable methods, such as dissolving in 9:1 methylene chloride-methanol (about 10 percent to about 20 percent w/w) and reprecipitating in ether. The polymeric product can then be dried to constant weight under high vacuum.

The hydrogenolysis methods described herein are the preferred means to prepare the polymers having pendent carboxylic acid groups. However, any other method that allows for the selective removal of a pendent carboxylate ester group is suitable for use in the preparation of these polymers. For example, iodotrimethylsilane can be used to selectively remove methyl ester pendent chains in the presence of ethyl ester pendent chains.

In some embodiments, the polymers are combined with a quantity of bioactive agent sufficient for effective treatment. The bioactive agent can be physically admixed, embedded in, or dispersed in the polymer matrix. Derivatives of bioactive agents can be attached to the polymer backbone by covalent bonds linked to the carboxylic acid pendent chain. This provides for the sustained release of the bioactive agent by means of hydrolysis of the covalent bond between the bioactive agent and the polymer backbone.

In addition, the pendent carboxylic acid groups of the polymers provide the polymers with a pH dependent dissolution rate. This further enables the polymers to be used to deliver bioactive agents to acidic environments of the body. The copolymers of these embodiments having a relatively high concentration of pendent carboxylic acid groups are stable and water insoluble in acidic environments but dissolve/degrade rapidly when exposed to neutral or basic environments. Also, the polymers having pendent carboxylic acid groups are more hydrophilic. Therefore, these polymers can be more readily absorbable under physiological conditions than polycarbonates and/or polyarylates that lack pendent carboxylic acid groups. As a result of the increased hydrophilicity of these polymers, they have a higher water uptake, and when the monomeric subunits having carboxylic acid groups predominate, they are more soluble in aqueous media. When the monomeric repeating subunits having pendent carboxylic acid groups do not predominate, the polymers can slowly dissolve in aqueous media with slower degradation. The dissolution/degradation rates are highly pH dependent.

In still further aspects, the pendent carboxylic acid groups on the polymers can function to regulate cell attachment, growth and migration on the polymer surfaces. The degree of copolymerization, that is, the ratio of pendent carboxylic acid groups to pendent ester groups, can be adjusted to provide polymeric materials that promote cellular attachment, migration, and proliferation, as well as polymers that inhibit attachment, migration, and proliferation.

II. Porous Scaffold Modifications

In some aspects, the biodegradable polymeric material can be provided as a porous scaffold useful for tissue engineering and tissue guided regeneration. Preferably, the biodegradable porous polymeric scaffolds are provided with a bimodal distribution of open pore sizes providing a high degree of interconnectivity, high internal surface area, and linearly aligned pores along the walls of the larger pores. The scaffolds can serve as both physical support and adhesive substrate for isolated cells during in vitro culturing and subsequent in vivo implantation. The scaffolds can be utilized to deliver cells to desired sites within the body, to define a potential space for engineered tissue, and/or to guide the process of tissue development. Suitable methods and porous scaffolds are described, for example, in U.S. Pat. No. 6,337,198 B1 (“Porous Polymer Scaffolds for Tissue Engineering,” Jan. 8, 2002), U.S. Pat. No. 6,103,255, and PCT/US99/08375 (WO 00/62829, Published Oct. 26, 2000).

Any of the biodegradable polymeric materials described herein can be suitably treated to provide porous polymeric scaffolds according to the invention. Preferred biodegradable porous scaffolds are provided having a substantially continuous polymer phase with a highly interconnected bimodal distribution of rounded large and small open pore sizes. Preferably, the large pores have an average diameter in the range of about 50 to about 500 microns, and the small pores have an average diameter less than about 20 microns, wherein the small pores are aligned in an orderly linear fashion within the walls of the large pores. In some embodiments, the small pores can be provided with an average diameter less than about 10 microns. The presence of the small pores, which form channels between the large pores, greatly enhances the pore interconnectivity. The resulting polymeric material has a porosity greater than about 90% and a high specific pore surface area in excess of 10 m²/g, or a porosity greater than about 95% and a specific pore surface area in excess of 20 m²/g.

The network of small pores is created in the walls of the large pores, and is well-oriented in a linear array. This provides surface patterning for guiding cell growth throughout the scaffold. This particular architecture also provides a large surface area and internal volume that is ideal for cell seeding, cell growth, and the production of extracellular matrices. Furthermore, the high interconnectivity of the pores allows for distribution of pores throughout the scaffold, transmission of cell-cell signaling molecules across the scaffolds, diffusion of nutrients throughout the structure, and the patterning of the surface to guide cell growth. The diameter and interconnecting structure of the pores promote vascularization and tissue ingrowth.

In preferred embodiments, the polymeric material is completely absorbed by the body over time, leaving only newly formed tissue in its place. However, such complete absorption is not required.

Thermally induced phase separation techniques are utilized to form the polymeric scaffolds. Depending upon such factors as thermodynamics, reaction kinetics, and the rate of cooling, phase separation will occur by solvent crystallization or liquid-liquid demixing. To form the porous scaffold structure, it is preferred to employ solvents and processing conditions under which solvent crystallization predominates.

For solvent crystallization to occur before liquid-liquid demixing, the selection of solvents and processing conditions is important. A mixture of two solvents is utilized, one in which the polymeric material is soluble (the “first solvent”) and one in which the polymeric material is insoluble (the “second solvent”). The first and second solvents must be miscible, and must form mixtures in which the polymeric material is soluble, despite its insolubility in the second solvent. Quantities of polymeric material, first solvent, and second solvent are selected to provide a uniform, homogeneous solution.

The first solvent preferably has a melting point in the range of about −40 to about 20° C. Within this range, at a high rate of cooling, crystallization is the favored phase separation mechanism. A melting point in the range of about −20° to about +20° C. is preferred. A solvent that ideally fits these requirements is 1,4-dioxane (melting point of 12° C. and a low crystallization energy). Solvents in which the polymeric material is insoluble that are suitable for use as the second solvent include water and alcohols such as, for example, methanol, ethanol, isopropanol, tert-butanol, and 1,3-propanediol. It is important that the polymeric material be soluble in the solvent mixture. One preferred pair of first and second solvents is 1,4-dixoane and water.

Typically, the ratio of first solvent to second solvent is in a range within which the polymeric material dissolves to form a homogeneous solution. The amount of the second solvent should thus be that quantity effective to induce phase separation on cooling, but less than the amount effective to induce phase separation before starting the procedure. The volume ratio of the first solvent to the total volume of solvent is typically in the range of about 1% to about 40% (v/v), or in the range of about 5% to about 15% (v/v).

The homogenous solution is then placed into a form containing water-soluble non-toxic particles that are insoluble in organic solvents. The particles are essentially any non-toxic biocompatible crystalline substance that is readily water-soluble and insoluble in organic solvents. Exemplary particles include biologically acceptable alkali metal and alkaline earth metal halides, phosphates, sulfates, and the like. Crystals of sugars can also be used, as well as microspheres of water-soluble polymers, or proteins, such as albumin. Sodium chloride is a particularly preferred particle. Particles are preferably selected having a diameter that is desired for the large pores of the bimodal distribution of pore sizes. Particle having a particle size diameter in the range of about 50 to about 500 microns are preferred, and diameters in the range of about 200 to about 400 microns are more preferred.

After diffusion of the polymeric material through the particles, the contents of the mold are rapidly cooled at a rate effective to induce crystallization of the first solvent before the onset of liquid-liquid demixing of the polymer solution. For example, the dish can be placed in liquid nitrogen or an equivalent cryogenic liquid and maintained in the liquid nitrogen for a rapid and complete quenching of the system.

The solvents are then sublimated from the polymer phase. The mold is placed in a vessel connected to a vacuum pump for the time needed for complete sublimation of the solvents. This step allows removal of the solvent by sublimation from the frozen materials so that it leaves a porous structure. The system is still frozen and the polymeric material does not relax during solvent removal.

The particles are removed by leaching with a solvent in which the particles are soluble and the polymeric material is insoluble. Exemplary solvents for leaching include the second solvent or water. The leaching solvent is changed several times to ensure complete removal of the particles. The resulting scaffolds are removed from the leaching solvent and dried to constant weight.

Suitable polymeric materials include any of the polymeric materials described herein.

The scaffolds can be further modified after fabrication. For example, bioactive agents can be provided to the scaffolds and coupled to the scaffolds through absorption or chemical bonding. In some embodiments, bioactive agent can be incorporated within the scaffold for subsequent release in a controlled fashion. The bioactive agent can be released by bioerosion of the polymer phase, and/or by diffusion from the polymer phase. Alternatively, the bioactive agent can migrate to the polymer surface of the scaffold structure, where it is active.

The polymeric material and the first and second solvents can be pre-blended before the bioactive agent is dissolved therein. Alternatively, the bioactive agent can be dissolved in the solvent in which it is most soluble, after which the first and second solvents and polymeric material are combined. As discussed herein, the bioactive agent can be provided in combination with any physiologically acceptable carrier, excipient, stabilizer, and the like. Such agents are known and will not be described further herein. The bioactive agent can also be coupled with agents to facilitate their delivery, such as antibodies, antibody fragments, growth factors, hormones, or other targeting moieties.

In still further embodiments, the bioactive agent can be covalently attached to polymeric materials having pendent free carboxylic acid groups. Such embodiments are discussed elsewhere herein.

The porous polymeric scaffolds can be characterized by scanning electron microscopy (SEM) and mercury porosimetry. The scaffolds can be shaped into articles as desired. The porous polymeric scaffolds can be utilized for tissue engineering and tissue-guided regeneration applications, including reconstructive surgery. The structure of the scaffold allows generous cellular ingrowth, reducing (or eliminating) the need for cellular preseeding. The polymeric porous scaffolds can also be molded to form external scaffolding for the support of in vitro culturing of cells for the creation of external support organs.

Optionally, the porous polymeric scaffold can be shaped, for example, by cutting with any suitable tool, to fit a desired end use application. Any crevices, apertures or refinements desired in the three-dimensional structure can be created by removing portions of the matrix with scissors, a scalpel, a laser, electrical discharge machining, or any other cutting instrument.

The scaffold can be used in transplantation as a matrix for dissociated cells to create a three-dimensional tissue or organ. Any type of cell can be added to the scaffold for culturing and potential implantation, including nerve cells, either as obtained from donors, from established cell culture lines, or autologous cells. In vitro culturing can optionally be performed prior to implantation. Alternatively, the scaffold can be implanted, allowed to vascularize, then cells provided into the scaffold (for example, by injection).

III. Additional Polymer to Control Bioactive Agent Release

In another aspect, the release of bioactive agent from the biodegradable polymeric material can be controlled by the inclusion of a second polymer within the polymeric matrix. The second polymer can be non-miscible with the basic biodegradable polymeric matrix material, so that phase-separated microdomains of the second polymer are formed in the bioactive agent/polymeric matrix. Accordingly, these aspects allow the bioactive agent release profile from the polymeric matrix to be modified and fine-tuned independent of the level of bioactive agent loading, the bioactive agent particle size, the distribution of bioactive agent particles within the polymeric matrix, or the degradation rate of the polymeric matrix material. These aspects of the invention will now be described in more detail.

Suitable polymeric formulations, and methods of preparing them, are described, for example, in U.S. Pat. No. 5,877,224 (“Polymeric Drug Formulations,” Mar. 2, 1999).

In the absence of a phase-disrupting second polymer, in most instances, the bioactive agent will be expressed at the matrix surface without being actually released from the polymeric matrix to any appreciable extent. Such a non-releasing formulation could be useful for some therapeutic applications of water-soluble bioactive agents where the bioactive agent is effective as a surface modifying agent.

The single-phase dispersions of these aspects of the invention are formed by simultaneously dissolving the bioactive agent and matrix polymeric material in an organic solvent system in which the bioactive agent and polymeric material are capable of forming a homogeneous solution. The homogeneous solution of bioactive agent and polymeric material can be used directly for the fabrication of coatings, tubes, filaments, or films by appropriate fabrication techniques. Alternatively, the homogeneous solution can be precipitated into a carefully selected non-solvent, resulting in the formation of an intimate, molecularly dispersed co-precipitate of bioactive agent and polymeric material.

Suitable polymeric materials include any of the biodegradable polymeric materials described herein. The polymeric material molecular weight will depend upon the requirements of the intended end use of the polymeric bioactive agent formulation. The polymeric material molecular weight is one factor to be considered for bioactive agent compatibility and an appropriate polymeric material molecular weight can be readily determined by one of ordinary skill in the art without undue experimentation.

A preferred tyrosine dipeptide derived poly(arylate) is poly(desaminotyrosyl-tyrosine hexyl ester adipate) (poly(DTH adipate)). Poly(DTH adipate) having a weight-average molecular weight in the range of about 80,000 to about 200,000 daltons is particularly preferred.

In addition to being chemically compatible with the biodegradable polymeric material, bioactive agents for use in the polymeric material of the invention must possess at least some solubility in the non-aqueous solvent systems utilized herein and must be chemically stable in the solvent systems. While the polymeric bioactive agent formulations are particularly well-suited for the delivery of peptide drugs, non-peptide drugs can be used as well. Exemplary suitable non-peptide drugs include natural and unnatural antibiotics, cytotoxic agents, and oligonucleotides.

Preferred polymeric bioactive agent formulations provide improved controlled release devices that show reproducible release profiles without significant burst and/or lag effects, or the premature deactivation of the bioactive gent during fabrication of the device. Peptide drugs suitable for formulation with the polymeric material compositions include natural and unnatural peptides, oligopeptides, cyclic peptides, library generated oligopeptides, polypeptides, and proteins, as well as peptide mimetics, so long as the specific bioactive agent moiety has some solubility in a single solvent or solvent mixture such that the bioactive agent and the water-insoluble polymeric material can form a homogeneous solution. The peptides can be obtained by some form of chemical synthesis or be naturally produced or be obtained by recombinant genetics, and can range in molecular weight as low as 200 daltons. Suitable peptide drugs include immunoglobulins and immunoglobulin fragments.

An important issue in formulating the biodegradable compositions is the evaluation of mutual miscibility between the polymeric material and the bioactive agent. In one aspect, the bioactive agent and the polymeric matrix must be miscible (blendable) in the solid state. The theoretical criteria for miscibility is a shift in the polymer glass transition temperature upon mixing of the bioactive agent with the polymeric material. An empirical criteria, as defined here within the context of the invention, is that upon solvent casting, extrusion, or compression molding a mixture of polymer and bioactive agent, a transparent device is obtained that is free of discrete bioactive agent particles visible to the naked eye. Transparency of the device indicates that the bioactive agent loaded polymeric matrix does not contain phase separated microdomains on the length scale of visible light, while a translucent device having a foggy, cloudy, or hazy appearance can be assumed to contain phase-separated microdomains on the length scale of visible light.

The polymeric material bioactive agent formulations can contain bioactive agent loadings from trace levels to about 60 percent by weight, or to about 55 percent by weight, or to about 50 percent by weight, or to about 45 percent by weight, or to about 40 percent by weight, or to about 35 percent by weight, or to about 30 percent by weight, or to about 25 percent by weight, or to about 20 percent by weight, although higher bioactive agent loadings can be useful in some instances. Preferably, the compositions contain a therapeutically or prophylactically effective amount of a bioactive agent.

The polymeric material bioactive agent formulations can optionally include a second polymer. In some aspects, the second polymer comprises a phase-disrupting polymer that is non-miscible with the polymeric material. One of ordinary skill in the art can readily select a second, phase-disrupting polymer that is non-miscible with the polymeric material without undue experimentation. Generally, since the polymeric material is water-insoluble, water-soluble polymers are good candidates for use as phase-disrupting polymers since these materials will usually be non-miscible with the polymeric matrix material. Water-solubility is additionally expected to be a favorable property for the phase disrupting polymer since it can enhance the observed release rate of the bioactive agent from the bioactive agent/polymeric material. Non-limiting examples of suitable second polymers include poly(alkylene oxides) such as poly(ethylene glycol), polysaccharides, poly(vinyl alcohol), polypyrrolidone, poly(acrylic acid), and its water-soluble derivatives such as poly(hydroxyethylmethacrylate), and the like. Non-polymeric materials that are non-miscible with the polymeric material can be used to form the phase-separated microdomains.

Generally, the precise molecular weight of the phase-disrupting polymer is not a critical parameter and can be determined on a trial and error basis, using phase-disrupting polymer preparations of different molecular weights and observing the resulting release profiles.

Molecular weights in the range of about 1,000 daltons to several hundred thousand daltons are useful. One of ordinary skill in the art can determine the optimal molecular weight of the phase-disrupting polymer needed to obtain the desired release profile for any given medical application. PEG is particularly well suited for use in combination with poly(DTH adipate) and PAI peptide. PEG having a weight-average molecular weight ranging from about 1,000 to about 2,000 daltons is particularly preferred. When PEG is used as the second phase-disrupting polymer, it should be present at a level in the range of about 2 and about 30 percent by weight. A level in the range of about 5 to about 15 percent by weight is preferred, with a level of about 10 percent by weight being most preferred.

As the concentration of the second, phase-disrupting polymer increases in the formulation, the rate of bioactive agent release from the polymer matrix will also increase, although this relationship is not linear. The bioactive release rate selected will depend upon such factors as the therapeutic dosage profile desired for the bioactive agent to be delivered.

The polymeric bioactive agent formulations are prepared by simultaneously dissolving the biodegradable polymeric material, bioactive agent, and second, phase-disrupting polymer in an organic solvent system capable of forming a homogeneous solution of the polymeric material, bioactive agent and second polymer. Typical solvent systems include one or more solvents selected from methanol, methylene chloride, ethanol, ethylene glycol, glycerol, tetrahydrofuran, ethyl acetate, acetonitrile, acetone, diisopropyl ether, methyl t-butyl ether, chloroform, carbon tetrachloride, dichloroethane, and water. Individual bioactive agent and polymer components must possess a solubility in at least one of the solvents of at least 1 g/l. The solvents can be pre-blended before the bioactive agent and the polymer(s) are dissolved therein. Alternatively, bioactive agent or polymer can be dissolved in the individual solvent in which it is most soluble, after which the solutions are combined to form a solvent system in which the bioactive agent and polymer are soluble.

The bioactive agent and polymers should be dissolved in the mixing solvents at a level preferably in the range of about 1 to about 30 percent by weight, and preferably in the range of about 5 and about 20 percent b weight. A concentration in the range of about 5 to about 10 percent by weight is preferred.

The relative solubilities of the bioactive agents and polymers intended for use in various organic solvents are well-known chemical properties. The selection of an organic solvent system in which a bioactive agent, a polymeric material, and a second, phase-disrupting polymer are forming a homogeneous solution at their respective concentrations can be readily determined without undue experimentation.

Briefly, using known solubility profiles of each individual component, one would first consider a simple mixture of each of the individual solvents. For example, if the bioactive agent has some solubility in acetone, the phase-disrupting polymer is soluble in methanol, and the polymeric material is soluble in methylene chloride, a mixture of acetone, methanol, and methylene chloride would be the initial starting point for the development of a solvent system that can dissolve all three of the components in a homogeneous solution. Next, hydrogen bonding effects, polarity effects, and common solvent effects are considered. Inspection of well-known solubility parameters also assists in finding suitable solvent mixtures for all three solutes. The identification of complex solvent mixtures for different solutes is a well-known task in formulation of numerous pharmaceutical products and can be readily accomplished by one skilled in the art.

The solution of bioactive agent and polymers is then precipitated into a non-solvent to form the single-phased dispersion of the bioactive agent in the polymeric material, including phase-separated microdomains caused by the presence of the second phase-disrupting polymer. The non-solvent should be miscible with the solvents that were used to dissolve bioactive agent and polymers. Using a non-solvent for the precipitation that is not fully miscible with each of the solvents used to dissolve bioactive agent and polymers carries the danger of obtaining a separation of the solvent mixture into two phases during the precipitation process. Although this can be acceptable in some cases, this is not the preferred mode of conducting the precipitation step. Exemplary non-solvents include ethers such as diethyl ether, diisopropyl ether, methyl t-butyl ether, and the like, as well as methyl ethyl ketone, acetone, ethyl acetate, acetonitrile, toluene, xylene, carbon tetrachloride, and the like. An excess of the non-solvent of at least 5-10 volumes compared to the volume of the dissolving solvents should be employed, and the non-solvent can be chilled as low as the freezing point of the non-solvent to promote the coprecipitation.

The coprecipitated bioactive agent-polymeric material is dried to remove any residual solvent and is then fabricated by known methods. Depending upon the thermal stability of the bioactive agent and the polymeric material, the articles can be shaped by conventional polymer-forming techniques such as extrusion, compression molding, injection molding, and the like.

As described in more detail below, the resulting polymeric material containing bioactive agent can be utilized as a coating (applied by conventional dipping, spray coating, and the like techniques), as a film placed in association with the device, and/or as a material used to form the implantable device itself.

The bioactive agents incorporated into the formulations can desirably further include agents to facilitate their delivery to a desired target, as long as the delivery agent meets the same eligibility criteria described above. Such agents include antibodies, antibody fragments, growth factors, hormones, or other targeting moieties, to which the bioactive agent can be coupled.

In addition to the above-described biodegradable polymeric materials, materials described in Applicant's copending Patent Application Ser. No. 60/583,171 (filed Jun. 24, 2004, entitled “Biodegradable Medical Device”) can be utilized. Materials described in that application include degradable polymers containing ester linkages (polyetherester copolymers, terephthalate esters with phosphorus-containing linkages, and segmented copolymers with differing ester linkages), as well as polycarbonate-containing random copolymers, and copolymers of these.

Device

One or more of the biodegradable metal or polymeric materials can be chosen to fabricate at least a portion of an implantable device in accordance with the invention. As discussed herein, various implantable device configurations are contemplated by the invention. The inventive biodegradable implantable devices are particularly suitable for treatment of limited access regions of the body. The configurations and characteristics of the implantable device will now be described in detail.

In one embodiment of the invention, the body member of the implantable device is the portion of the controlled release device that is inserted into a patient. The body member can be described as including a proximal end (which is located, upon implantation, towards the exterior of the body), a distal end (which is located, upon implantation, towards the interior of the body), and a longitudinal axis. According to these embodiments, at least a portion of the body member is inserted into a patient's body. For example, in some embodiments, it can be preferable to position less than 100% of the body member inside the patient's body. The amount of the body member positioned within the body can be determined by the interventionalist, based upon such factors as desired treatment parameters, the particular configuration of the device, the implantation site, and the like.

In some aspects, the invention provides biodegradable implantable devices that are composed of a body member having a direction of extension, and one or more deviations from that direction of extension. In some embodiments, the body member further includes a direction of extension, and in preferred embodiments, at least a portion of the body member deviates from the direction of extension. In preferred embodiments, the body member includes at least two, three, four, five, six, seven, eight, nine, ten, or more deviations from the direction of extension. In some alternative embodiments, where the body does not include multiple deviations from the direction of extension, the body member can be provided in a “J” or a hook-type configuration. Illustrative embodiments are described in U.S. Publication No. 2005/0019371 A1 (Anderson et al., “Controlled Release Bioactive Agent Delivery Device, filed Apr. 29, 2004), U.S. Pat. No. 6,719,750 B2 (Varner et al., “Devices for Intraocular Drug Delivery,” filed Jun. 22, 2001), U.S. Publication No. 2004/0133155 A1 (Varner et al., “Devices for Intraocular Drug Delivery,” filed Dec. 19, 2003), U.S. Publication No. 2005/0059956 A1 (Varner et al., “Devices for Intraocular Drug Delivery,” filed Apr. 12, 2004), and U.S. Publication No. 2003/0014036 A1 (Varner et al., “Reservoir Device for Intraocular Drug Delivery,” filed Jun. 12, 2002), and related applications.

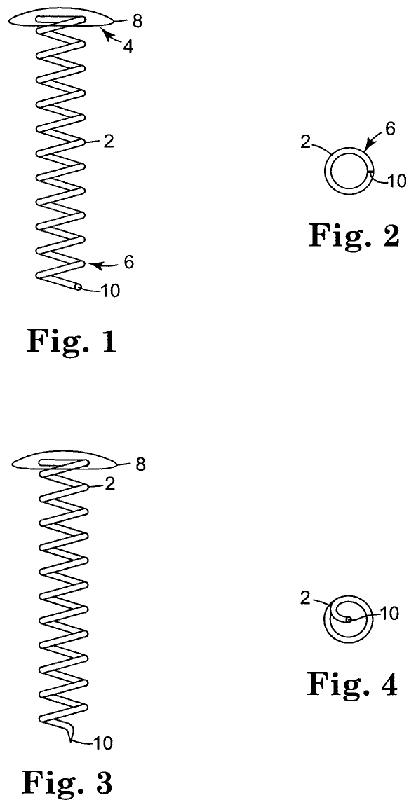

The deviations from the direction of extension can be provided in any suitable configuration. Exemplary embodiments of such deviations will be described herein for illustrative purposes only, and without intending to be bound by any particular embodiment described herein. The deviations need not be rounded or arcuate. For example, in some embodiments, the body member is provided with a Z-shaped configuration, such that the deviations are angular. Moreover, the deviations need not be in a regular pattern, but can alternatively be provided in a random manner, such that the body member contains random curls or turns. In some embodiments, the deviations are provided in a patterned configuration about the longitudinal axis. Examples of these patterned embodiments include coils, spirals, or patterned Z-shaped turns in the body. Alternatively, the deviations can be provided in a random or non-patterned configuration about the longitudinal axis. According to these particular non-patterned embodiments, the distance of the individual deviations from the longitudinal axis to the outermost periphery of the body member can be selected to provide a desired overall profile of the body member, depending upon the application of the device. For example, it can be desirable, in some applications, to provide an overall profile of the body member having an hourglass shape, alternating ring circumference shapes, and the like. Reference is made to FIGS. 1-4, wherein FIG. 1 illustrates an implant including a body member 2 having a proximal end 4 and a distal end 6. FIG. 1 illustrates the body member 2 in a coil configuration.

In some embodiments, the deviations from the direction of extension can be provided in the form of rings. Such individual rings can be concentric (that is, having a common axis, or being coaxial about the longitudinal axis) or eccentric (deviating from a circular path). According to these embodiments, the individual rings are noncontiguous along the body member length, thereby forming individual ribs at positions along the direction of extension of the body member.

Preferred configurations of the body member are coiled or spiral. Generally, in a coil configuration, the individual rings of the coil rotate about the longitudinal axis, and the overall coil is substantially symmetrical about the longitudinal axis. A preferred coil is composed of multiple rings that are substantially similar in circumference along the length, from proximal to distal, of the device. In some preferred embodiments, the rings form a spiral pattern, wherein the circumference of the rings changes over the length of the device. Preferably, the circumference of the rings decreases toward the distal direction of the device, so that the largest ring circumference is located at the proximal region of the device, and the smallest ring circumference is located at the distal region of the device.

Inclusion of deviating portions of the body member provides an increased surface area for delivery of a bioactive agent to an implantation site as compared to a linear device having the same length and/or width. This can provide advantages during use of the device, since this configuration allows a greater surface area to be provided in a smaller length and/or width of the device. For example, in some applications, it can be desirable to limit the length of the device. For example, as will be discussed in more detail herein, it is desirable to limit the length of implants in the eye to prevent the device from entering the central visual field of the eye and to minimize risk of damage to the eye tissues. By providing a body member that has at least a portion of the body member deviating from the direction of extension, the device of the invention has greater surface area (and thus can hold a greater volume of bioactive agent) per length of the device without having to make the cross section of the device, and thus the size of the implantation incision, larger.

Still further, in preferred embodiments, the shape of the body member can provide a built-in anchoring system that reduces unwanted movement of the device and unwanted ejection of the device out of the patient's body, since the shape of the body member requires manipulation to remove it from an incision. For example, for a coil-shaped body member, the device would require twisting, and a Z-shaped body member would require back and forth movement, to remove the device from the implantation site. According to some preferred embodiments, the device does not require additional anchoring mechanisms (such as suturing) to the body tissues, as a result of the self-anchoring characteristics of the device itself. As described in more detail herein, inclusion of a cap or other anchoring mechanism on the device can provide further anchoring features of the device.

In some embodiments, when the body member includes two or more deviations from the direction of extension, the spacing of the individual deviations can be selected to provide an optimum combination of such features as increased surface area for bioactive agent delivery, overall dimensions of the device, and the like. For example, when the body member is provided in the form of a coil that includes two or more deviations from the direction of extension, the distance between the individual coils can be selected to be equal to or greater than the diameter of the material forming the body member. In some aspects, the distance between individual coils can be selected to provide a device that can be coated with biodegradable polymeric material. For example, if the distance between coils is less than the diameter of the material forming the body member, the amount of coatable surface area of the body member can decrease, since it can be more difficult to access portions of the surface area of the body member with the biodegradable polymeric coatings. By considering the accessibility of the device surface for coating applications, the amount of biodegradable polymeric material that can be provided as a coating can be controlled. In one illustrative embodiment of this aspect of the invention, the body member is formed of a material having a diameter of 0.5 mm, and the distance between each coil of the body member is at least 0.5 mm. These principles can be applied to any configuration of the body member and is not limited to coiled configurations.

In alternative embodiments, the implantable device can be provided in a linear (for example, rod-shaped, wire, filament, or film) configuration, or as a rounded (for example, disc-shaped, bead, or oblong rounded shaped) configuration for implantation into the body. Illustrative embodiments are described, for example, in U.S. Patent Publication No. 2002/0198511 A1 (Varner et al., “Method and Device for Subretinal Drug Delivery,” filed Jun. 22, 2001), and PCT Publication No. WO 2004/028477 (de Juan et al., “Method for Subretinal Administration of Therapeutics Including Steroids; Method for Localizing Pharmacodynamic Action at the Choroid and the Retina; and Related Methods for Treatment and/or Prevention of Retinal Diseases,” filed Sep. 29, 2003); and related applications.

The implantable device can be placed on or near a site within the body to be treated. For example, for treatment of a scleral site, the device can be placed against the sclera, while treatment of the retina can be accomplished by placement of the device on or near the retina (for example, in the sub-retinal space). The implantable device can be inserted into the vitreal chamber (as described above for the device containing deviations from the direction of extension). Alternatively, the implantable device can be implanted in regions of the eye outside the vitreal chamber. According to these latter embodiments, the implantable device can be implanted at any desired location of the eye (such as the sclera or cornea), or the intermediate layer (the anterior layer (including iris and ciliary body) or posterior (including the choroid). Likewise, the implantable devices according to the invention can be placed within any desirable chamber of the eye, including the anterior chamber, posterior chamber, and/or vitreous chamber. Alternatively, the devices can be provided at a surface of the eye, for example, when treating a condition that affects a surface such as the retina, lens, and the like.

The particular configuration of the implantable device (linear, rounded, coiled, or the like) can be determined upon such factors as the implantation site, duration of treatment, amount of bioactive agent to be delivered to the treatment area, and the like. One of skill in the art, upon review of this disclosure, will readily appreciate the virtually limitless configurations that can be achieved utilizing the polymeric materials described herein.

The overall dimensions of the implantable device can be selected according to the particular application. For example, the length and/or width of the device can be selected to accommodate the particular implantation site. Some factors that can affect the overall dimensions of the implantable device include the potency of any bioactive agent to be delivered (and thus the volume of bioactive agent required, which impacts the surface area of the device, as discussed herein), the location of the implantation site within the body (for example, how far within the body the implantation site is located), the size of the implantation site (for example, a small area such as the eye or inner ear, or a larger area, such as a joint or organ area), the tissue surrounding the implantation site (for example, vascular tissue or hard, calcinous tissue, such as bone), and the like.

By way of example, when the implantable device is used to deliver bioactive agent(s) to the eye, the device is preferably designed for insertion through a small incision that requires few or no sutures for scleral closure at the conclusion of the surgical procedure. As such, the device is preferably inserted through an incision that is no more than about 1 mm in cross-section, for example, in the range of about 0.25 mm to about 1 mm in diameter, preferably in the range of about 0.25 mm to about 0.5 mm in diameter. As such, the cross-section of the material forming the body member is preferably no more than about 1 mm, for example, in the range of about 0.25 mm to about 1 mm in diameter, preferably in the range of about 0.25 mm to about 0.5 mm in diameter. These dimensions are particularly useful when providing a device for vitreal delivery of bioactive agent, such as the implant depicted in FIGS. 1-5. When the material forming the body member is not cylindrical, the largest dimension of the cross-section can be used to approximate the diameter of the body member for this purpose, for example, when the body member cross-section is square.

When used to deliver bioactive agent(s) to the eye, and in particular the vitreal chamber of the eye, the body member of the controlled release device preferably has a total length from its proximal end to its distal end that is less than about 1 cm, for example, in the range of about 0.25 cm to about 1 cm. Upon implantation, the body member is positioned within the eye, such that the portion of the controlled delivery device that delivers bioactive agent to the eye chamber is positioned in or near the posterior segment of the eye. When the controlled delivery device includes a cap, the cap is preferably provided with a thickness of less than about 1 mm, more preferably less than about 0.5 mm. According to this particular embodiment, the total length of the controlled delivery device is less than about 1.1 cm, preferably less than about 0.6 cm.

In general, materials used to fabricate the body member of the implantable device are not particularly limited. In some embodiments, the body member can be fabricated of a flexible material, so that small movements of the controlled delivery device will not be translated to the implantation site. In some embodiments, as described in further detail herein, it can be preferable to fabricate a portion (such as the distal end) of the body member of a rigid, non-pliable material. For example, when the device is designed for implantation in the eye, it is preferable to fabricate the device of a rigid material, to provide improved implant/explant characteristics to the device. In some embodiments, as described herein, it can be preferable to fabricate the body member of a material having shape memory and/or superelastic characteristics.

Portions of Device Fabricated of Nondegradable Materials

As described herein, the implantable device can include nondegradable components. In some embodiments, the body member can be fabricated from any suitable nondegradable material used to manufacture medical devices, such as, for example, stainless steel (for example, 316L); platinum; titanium; and gold; and such alloys as cobalt chromium alloys, nitinol, or the like. In further embodiments, suitable ceramics can be used to fabricate the body member, such as, for example, silicon nitride, silicon carbide, zirconia, alumina, glass, silica, sapphire, and the like. In still further embodiments, the body member can be fabricated of a suitable composite material, such as composite materials commonly used to fabricate implantable devices. Such composite materials can, in some embodiments, provide such advantages as increased strength of the material, as well as increased flexibility. Examples of suitable composite materials include polymers or ceramics, (such as polymethylmethacrylate bone cement (PMMA), dental polymer matrix (such as crosslinked methacrylate polymers), and glass-ceramics), high density polyethylene (HDPE), ultra high molecular weight polyethylene (UHMWPE), reinforced with fibers or particulate material (such as carbon fibers, bone particles, silica particles, hydroxyapatite particles, metal fibers or particles, or zirconia, alumina, or silicon carbide particles). Nano-composite materials are also contemplated.

In one embodiment, a portion of the implantable device is fabricated of a nonbiodegradable polymer. Such nonbiodegradable polymers are well known and can include, for example, oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; and vinyls such as ethylene, propylene, styrene, vinyl chloride, vinyl acetate, and vinylidene difluoride. Examples of condensation polymers include, but are not limited to, nylons such as polycaprolactam, polylauryl lactam, polyhexamethylene adipamide, and polyhexamethylene dodecanediamide, as well as polyurethanes, various types of nondegradable polycarbonates, polyamides, polysulfones, poly(ethylene terephthalate), polydimethylsiloxanes, and polyetherketone. Other suitable nonbiodegradable polymers include silicone elastomers; silicone rubber; polyolefins such as polypropylene and polyethylene; homopolymers and copolymers of vinyl acetate such as ethylene vinyl acetate 2-pyrrolidone copolymer; polyacrylonitrile butadiene; fluoropolymers such as polytetrafluoroethylene and polyvinyl fluoride; homopolymers and copolymers of styrene acrylonitrile; homopolymers and copolymers of acrylonitrile butadiene styrene; polymethylpentene; polyimides; natural rubber; polyisobutylene; polymethylstyrene; latex; and other similar nonbiodegradable polymers.

In some embodiments, when the implantable device is provided with a configuration having a direction of extension and deviations from that direction of extension (such as the configuration illustrated in FIGS. 1-5), at least a portion of the body member can deviate from the direction of extension prior to, during, and after insertion of the device in the body. Alternatively, the device can be fabricated of a material having shape memory and/or superelastic characteristics that allow the device to be deformed into a configuration that is more easily inserted into the body. In one such embodiment, for example, the body member can be deformed into a substantially linear configuration, for insertion into the body. According to this particular embodiment, the body member can return to its original shape after it is inserted into the body. In this embodiment, the body member of the device has a “memory shape” that it will assume under certain conditions. When the interventionalist desires to implant the device into the body, the interventionalist can deform the device into a substantially linear shape for insertion of the device through an incision the size of the cross section of the linear shaped device. Upon implantation of the device into the body, the device can then resume its memory shape. Preferably, the overall dimensions of the controlled delivery device (the maximum length and width) according to these shape memory embodiments do not significantly change by virtue of utilization of the shape memory material and deformation of the body member for implantation and/or explantation of the device in the body.

Preferably, the controlled delivery device of the invention takes advantage of the material properties of the body member (for example, superelastic properties) to extend the body member into a linear shape. Once placed at the implantation site in an unconstrained form, the body member can resume its memory shape.

Device Configuration

The distal end of the body member is typically the first end of the device to be inserted into a body. Thus, the distal end 6 of the body member 2 can be fabricated to include any suitable configuration, depending upon the application of the device and the site of the body at which the device is to be implanted. For example, in some embodiments, the distal end 6 can include a tip 10 that is blunt or rounded (as illustrated in FIG. 1). In preferred embodiments, the distal end 6 of the body member 2 is configured to pierce the body during implantation of the device into the body. For example, the distal end 6 of the body member 2 can include a sharp or pointed tip 10 (as illustrated in FIG. 3). In one preferred embodiment, the distal end 6 of the body member 2 has a ramp-like angle. Preferably, the device according to this embodiment can be utilized to make an incision in the body, rather than requiring separate equipment and/or procedures for making the incision site. If the distal end 6 of the body member 2 is used to pierce the body during insertion, at least the distal end 6 is preferably fabricated of a rigid, non-pliable material suitable for piercing the body. Such materials are well known and can include, for example, polyimide and similar materials. In one such preferred embodiment, the distal end 6 of the body member 2 is utilized to pierce the eye for insertion of the controlled delivery device in the interior of the eye.

The body member can be fabricated from a solid material (a material that does not contain a lumen) or a material containing a lumen, as desired. The choice of a solid or lumen-containing material is not critical to the invention and can be determined based upon availability of materials and processing considerations.

When included, the lumen(s) can extend along the length of the body member or only a portion of the length of the body member, as desired. In some embodiments, the lumen(s) can serve as a delivery mechanism for delivery of a desired substance to the implantation site. The substance delivered via the lumen can comprise any of the bioactive agents described herein. The substance delivered via the lumen can be the same or different bioactive agent(s) from that included in the biodegradable polymeric material. Further, the substance can be provided in addition to the bioactive agent of the biodegradable polymeric material, or in place of the bioactive agent. For example, in one embodiment, one or more substances can be delivered via the lumen, and one or more bioactive agents can be provided to the implantation site from the polymeric coated composition.

When the lumen is to be provided with a substance, the lumen can be filled with the desired substance prior to inserting the device into the body, or after the device has been inserted into the body. When it is desired to fill the device with the substance after insertion into the body, a port can be provided near the proximal end of the body member for such purpose. The port is in fluid communication with the lumen(s) of the body member and can also be used for refilling the device with the substance after implantation, when desired.

When the device includes a port, the port is preferably designed such that the needle of an injection mechanism (for example, a syringe) can be inserted into the port and the substance to be included in the lumen injected by the injection mechanism. Thus, the substance can travel through the port and into the lumen(s) of the body member. The port preferably forms a snug seal about the needle of the injection mechanism to prevent leakage of the substance out of the port around the injection mechanism and to provide sterile injection of substance into the lumen(s). If desired, fittings or collars (not shown), through which an injection mechanism can be inserted and which form a snug seal about the injection mechanism, can be mounted on the port. Upon injection of the substance into the delivery device, the needle of the injection mechanism is removed from the port and the port sealed. Sealing can be accomplished by providing a removable cover (not shown) on the port that can be removed for injection of the substance and replaced when the substance has been injected. In a preferred embodiment, the port is fabricated of a self-sealing material through which the injection mechanism can be inserted and which seals off automatically when the injection mechanism is removed. Such self-sealing materials are known and include, for example, silicone rubber, silicone elastomers, polyolefin, and the like.

In further embodiments, when the device includes more than one lumen, the device can include more than one port. For example, each lumen can be in fluid communication with a plurality of ports. These ports are similar to the single port described above. If desired, the lumens and ports can be arranged such that each lumen can be filled with a different substance through a corresponding port (for example, each lumen has its own dedicated port). It can be desirable to include more than one lumen when it is desirable to deliver more than one additional substance to the implantation site.

In embodiments where it is desired to deliver one or more additional substances to the implantation site via one or more lumens, the individual lumens can include one or more apertures to allow such delivery. In one embodiment, such apertures are provided at the distal end of the device. In other embodiments, the apertures are provided along the length of the body member. The number and size of the apertures can vary depending upon the desired rate of delivery of the substance (when provided) and can be readily determined by one of skill in the art. The apertures are preferably designed such that the substance to be delivered is slowly diffused rather than expelled as a fluid stream from the device. For example, when the device is implanted in the eye, it is preferable to deliver the substance through slow diffusion rather than expulsion of the substance as a fluid stream, which can damage the delicate tissues of the eye. In some embodiments, the biodegradable polymeric coating in contact with the body can provide a particular porosity to the substance and can assist in controlling the rate of diffusion of the substance from the lumen. When included in the device, the particular location of the apertures can be situated so as to deliver the substance at a particular location once the device is implanted into the body.

In another embodiment, when the body member includes a lumen for delivery of an additional substance to the implantation site, the material forming the body member can be chosen to be permeable (or semi-permeable) to the substance to be delivered from the lumen. According to this particular embodiment, the body member material can be chosen depending upon the particular application of the device and the substance to be delivered and can be readily determined by one of skill in the art. Examples of suitable permeable materials include polycarbonates, polyolefins, polyurethanes, copolymers of acrylonitrile, copolymers of polyvinyl chloride, polyamides, polysulphones, polystyrenes, polyvinyl fluorides, polyvinyl alcohols, polyvinyl esters, polyvinyl butyrate, polyvinyl acetate, polyvinylidene chlorides, polyvinylidene fluorides, polyimides, polyisoprene, polyisobutylene, polybutadiene, polyethylene, polyethers, polytetrafluoroethylene, polychloroethers, polymethylmethacrylate, polybutylmethacrylate, polyvinyl acetate, nylons, cellulose, gelatin, silicone rubbers, porous fibers, and the like.

Alternatively, when the body member is fabricated from a biodegradable polymeric material described herein, the substance can be delivered from the lumen as the polymeric material degrades and/or by passage through the polymeric material (such as passage through pores in the polymeric material and/or by diffusion through the polymeric material itself).

According to these particular embodiments, the material used to fabricate the body member can be chosen to provide a particular rate of delivery of the substance, which can be readily determined by one of skill in the art. Further, the rate of delivery of the substance can be controlled by varying the percentage of the body member formed of the permeable (or semi-permeable) material. Thus, for example, to provide a slower rate of delivery, the body member can be fabricated of 50% or less permeable material. Conversely, for a faster rate of delivery, the body member can be fabricated of greater than 50% of permeable material. When one or more portions of the body member, rather than the whole body member, is fabricated of a permeable or semi-permeable material, the location of the permeable or semi-permeable material can be situated so as to deliver the substance at a particular location once the device is implanted at the implantation site.

In preferred embodiments, the body member can be fabricated in a way that further increases the surface area of the body member, preferably without increasing the overall dimensions of the device. Such increased surface area can provide enhanced bioactive agent delivery (from a biodegradable polymeric coating and/or device body that is fabricated from biodegradable polymeric material). For example, in one embodiment, the device can be fabricated of multiple strands of material that are entwined or twisted around each other to form the body member (for example, multiple strands of wire can be twisted around each other to form the body member). According to these particular embodiments, any number of individual strands can be utilized to form the body member, for example, 2, 3, 4, or more strands. The number of individual strands combined (as by twisting or other manipulation) to form the body member can be selected depending upon such factors as, for example, the desired diameter of the material forming the body member and/or the overall body member diameter, the desired flexibility or rigidity of the device during insertion and/or implantation, the size of the implantation, the desired incision size, the material used to form the body member, and the like.

When a coating is applied to these types of body members, provision of a biodegradable polymeric coating to the body member according to these embodiments can be achieved in any desirable manner. For example, each individual strand can be provided with a biodegradable polymeric coating prior to twisting the strands to form the body member. Alternatively, the individual, uncoated, strands can be twisted to form the body member, and the formed body member can be provided with a biodegradable polymeric coating.

In another embodiment, the surface area of the body member can be increased by including surface configurations on the body member. The increased surface area increases area that can be provided with biodegradable polymeric material as a coating. According to these embodiments, any suitable type of surface configuration can be provided to the body member, such as, for example, dimples, pores, raised portions (such as ridges or grooves), indented portions, and the like. Surface configuration can be accomplished by roughening the surface of the material used to fabricate the body member. In one such embodiment, the surface of the body member is roughened using mechanical techniques (such as mechanical roughening utilizing such material as 50 μm silica), chemical techniques, etching techniques, or other known methods. In other embodiments, surface configuration can be accomplished by utilizing a porous material to fabricate the body member. Examples of porous material are described elsewhere herein. Alternatively, materials can be treated to provide pores in the material, utilizing methods well known in the art. In still further embodiments, surface configuration can be accomplished by fabricating the body member of a machined material, for example, machined metal. The material can be machined to provide any suitable surface configuration as desired, including, for example, dimples, pockets, pores, and the like.

In still further embodiments, increased device surface area can be provided by utilizing a body member configured as a threaded shaft that is tapered or untapered, as desired. Such threaded shaft embodiments are similar to a typical wood screw. The threaded shaft can be fabricated using any suitable techniques, such as molding or machining the threads of the shaft. Further, the threading on the shaft can be a continuous spiral thread that runs continually from the proximal to the distal end of the body member, or the threading can be provided as noncontiguous rings about the body member. Although these particular embodiments can require a larger incision site for implantation of the device in a patient, in some applications, the increased surface area provided by the threaded shaft (discussed in more detail herein) can outweigh the larger incision required.

In preferred embodiments, surface configuration of the body member can provide advantages, such as, for example, increased surface area of the body member for application of a biodegradable polymeric coating (when applied), increased durability of the device, increased tenacity of the biodegradable polymeric coating to the body member (for example, by virtue of a roughened surface, increased surface area for adherence, and the like), and the like.

The body member can include surface configurations along its entire length, or only a portion of the length of the body member, as desired.

The cross-sectional shape of the body member can be selected depending upon the desired application. Thus, the cross-sectional shape of the body member can be circular, square, rectangular, octagonal, or other desired cross-sectional shapes.

One embodiment of an implantable device can include a cap 8 positioned at the proximal end 4 of the body member 2, as illustrated in FIGS. 1 and 3. When included in the device, the cap 8 can assist in stabilizing the device once implanted in the body, thereby providing additional anchoring features of the device. Preferably, the device is inserted into the body through an incision until the cap 8 abuts the incision on the exterior of the body. If desired, the cap 8 can then be sutured to the body at the incision site to further stabilize and prevent the device from moving once it is implanted in its desired location. When the device is implanted in the eye, for example, the device can be inserted into the eye through an incision until the cap 8 abuts the incision. If desired, the cap 8 can then be sutured to the eye, to provide further stabilization as discussed above.

The overall size and shape of the cap is not particularly limited, provided that irritation to the body at the incision site is limited. Preferably, the cap is sized such that it provides a low profile. For example, the dimensions of the cap are preferably selected to provide a small surface area to accomplish such desired features as additional anchoring characteristics of the device, without substantially increasing the overall profile of the device upon implantation. In some embodiments, for example, the cap can be covered by a flap of tissue at the incision site upon implantation, to further reduce potential irritation and/or movement of the device at the implantation and/or incision sites. One illustrative example described in more detail elsewhere herein is the covering of the cap with a scleral cap upon implantation of the device in the eye.

Further, the cap can be of any shape, for example, circular, rectangular, triangular, square, and the like. In order to minimize irritation to the incision site, the cap preferably has rounded edges. The cap is designed such that it remains outside the implantation site and, as such, the cap is sized so that it will not pass into the implantation site through the incision through which the device is inserted.

As described herein, inclusion of a cap in the device can provide additional anchoring features to the device itself. However, in some embodiments, it can be desirable to further secure the device to provide additional anchoring or securing features at the implantation site. Thus, when desired, the cap can be further designed such that it can be easily sutured or otherwise secured to the surface surrounding the incision and can, for example, contain one or more holes (not shown) through which sutures can pass.

The materials used to fabricate the cap are not particularly limited and include any of the materials previously described for fabrication of the body member. In one embodiment, the materials used to fabricate the cap are insoluble in body fluids and tissues with which the device comes in contact (thus, in this embodiment, the materials are nondegradable to allow the cap to perform an anchoring function). Alternatively, the cap is fabricated from materials that are biodegradable, yet at a rate that is slower than other degradable portions of the device (thus, in this embodiment, the anchoring function is provided by the cap for the time period required, after which it degrades as well). Further, it is preferred that the cap is fabricated of a material that does not cause irritation to the portion of the body that it contacts (such as the area at and surrounding the incision site). For example, when the device is implanted into the eye, the cap is preferably fabricated from a material that does not cause irritation to the portion of the eye that it contacts. As such, preferred materials for this particular embodiment include, by way of example, various polymers (such as silicone elastomers and rubbers, polyolefins, polyurethanes, acrylates, polycarbonates, polyamides, polyimides, polyesters, polysulfones, and the like), as well as metals (such as those described previously for the body member).

In some embodiments, the cap can be fabricated from the same material as the body member. Alternatively, the cap can be fabricated from a material that is different from the body member. The cap can be fabricated separately from the body member, and subsequently attached to the body member, using any suitable attachment mechanism (such as, for example, suitable adhesives or soldering materials). For example, the cap can be fabricated to include an aperture, into which the body member is placed and thereafter soldered, welded, or otherwise attached. In alternative embodiments, the cap and body member are fabricated as a unitary piece, for example, utilizing a mold that includes both components (the body member and cap) of the device. The precise method of fabricating the device can be chosen depending upon such factors as availability of materials and equipment for forming the components of the device.

In some embodiments, the cap can be provided with a biodegradable polymeric coating. According to these particular embodiments, the biodegradable polymeric coating provided in connection with the cap can be the same as, or different from, the biodegradable polymeric coating provided in connection with the body member. For example, the particular bioactive agent included in the biodegradable polymeric coating for the cap can be varied to provide a desired therapeutic effect at the incision site. Exemplary bioactive agents that could be desirable at the incision site include antimicrobial agents, anti-inflammatory agents, and the like, to reduce or otherwise control reaction of the body at the incision site.

In some embodiments, the cap can include a polymeric coated composition that is the same as the polymeric coated composition provided in connection with the body member. According to these embodiments, the biodegradable polymeric coating can be applied in one step to the entire controlled delivery device (body member and cap), if desired. Alternatively, the biodegradable polymeric coating can be applied to the cap in a separate step, for example, when the cap is manufactured separately, and subsequently attached to the body member.

The polymeric coated composition is provided in contact with at least a portion of the body member of the device. In some embodiments, for example, it can be desirable to provide the polymeric coated composition in contact with the entire surface of the body member. Alternatively, the polymeric coated composition can be provided on a portion of the body member (such as, for example, an intermediate portion of the body member located between the proximal and distal ends thereof). In some preferred embodiments, for example, it can be desirable to provide the polymeric coated composition in contact with a portion of the body member that does not include a sharp distal tip of the body member. This can be desirable, for example, to reduce risk of delamination of the polymeric coated composition at the sharp tip and/or to maintain the sharpness of the tip. The amount of the body member that is in contact with the coated composition can be determined by considering such factors as the amount of bioactive agent to be provided at the implantation site, the choice of biodegradable polymeric material, the characteristics of the implantation site, risk of delamination of the polymeric coated composition, and the like. For example, in some embodiments, it can be desirable to provide the polymeric coated composition on portions of the body member other than the proximal and distal ends of the device, so as to reduce risk of delamination upon implant and/or explant of the device. Optionally, such delamination can also be minimized, in some embodiments, by providing a stepped coating thickness, such that the coating thickness decreases towards the proximal and/or distal ends of the body member. In still further optional embodiments, the body member can be provided with a polymeric coated composition at its distal and/or proximal ends that differs from the composition of the coating at other portions of the body member. One example of such an embodiment includes a body member having a lubricious coating at the distal and/or proximal end of the body member, with a different polymeric coated composition in the intermediate portion of the body member that is located between the proximal and distal ends of the body member. Utilizing the concepts described herein, one of skill in the art can determine the amount of body member to be provided in contact with the polymeric coated composition, and/or the composition of polymeric coated composition provided at one or more distinct regions of the body member, as desired.

Bioactive Agents

In preferred embodiments, the polymeric material comprises a bioactive agent. For purposes of the description herein, reference will be made to “bioactive agent,” but it is understood that the use of the singular term does not limit the application of bioactive agents contemplated, and any number of bioactive agents can be provided using the teaching herein. As used herein, “bioactive agent” refers to an agent that affects physiology of biological tissue. Bioactive agents useful according to the invention include virtually any substance that possess desirable therapeutic characteristics for application to the implantation site.

Exemplary bioactive agents include, but are not limited to, thrombin inhibitors; antithrombogenic agents; thrombolytic agents (such as plasminogen activator, or TPA: and streptokinase); fibrinolytic agents; vasospasm inhibitors; calcium channel blockers; vasodilators; antihypertensive agents; clotting cascade factors (for example, protein S); anti-coagulant compounds (for example, heparin and nadroparin, or low molecular weight heparin); antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, α-methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopolamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl, betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, anti-VEGF antibody such as anti-VEGF fragment—ranibizumab (Lucentis)); dopamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like.

Another group of useful bioactive agents are antiseptics. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Another group of useful bioactive agents are enzyme inhibitors. Examples of enzyme inhibitors include chrophonium chloride, N-methylphysostigmine, neostigmine bromide, physostigmine sulfate, tacrine HCL, tacrine, 1-hydroxymaleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor 1, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−)deprenyl HCl, iproniazid phosphate, 6-MeO-tetrahydro-9H-pyrido-indole, nialamide, pargyline HC1, quinacrine HCl, semicarbazide HCl, tranylcypromine HC1, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+) p-aminoglutethimide tartrate, S(−)₃-iodotyrosine, alpha-methyltyrosine, L(−)alpha methyltyrosine, D,L(−)cetazolamide, dichlorophenamide, 6-hydroxy-2-benzothiazolesulfonamide, and allopurinol.

Another group of useful bioactive agents are anti-pyretics and antiinflammatory agents. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

The particular bioactive agent, or combination of bioactive agents, can be selected depending upon one or more of the following factors: the application of the controlled delivery device, the medical condition to be treated, the anticipated duration of treatment, characteristics of the implantation site, the number and type of bioactive agents to be utilized, and the like.

The chemical stability of bioactive agents with polymeric materials can be readily determined by one of ordinary skill in the art without undue experimentation. Typically, compatibility studies involve fabrication of bioactive agent-loaded polymeric matrices, followed by the evaluation of polymer molecular weight, bioactive agent purity, and the identification of any newly formed chemical species by HPLC, FT-IR, mass spectrometry, or other analytical techniques.

The concentration of the bioactive agent in the polymeric material can be provided in the range of about 0.01% to about 90% by weight, based on the weight of the final biodegradable polymeric coating. Preferably, the bioactive active agent is present in the polymeric material in an amount in the range of about 75% by weight or less, preferably about 50% by weight or less. The amount of bioactive agent in the polymeric material can be in the range of about 1 μg to about 10 mg, or about 100 μg to about 1500 μg, or about 300 μg to about 1000 μg.

Biodegradable Polymeric Material as Coating

When utilized as a coating, the biodegradable polymeric material can be applied to the controlled delivery device using any suitable methods. For example, the biodegradable polymeric material can be applied by dipping, spraying, and other common methods for applying coating compositions to implantable devices. The suitability of the biodegradable polymeric material for use on a particular material, and in turn, the suitability of the coated polymeric material, can be evaluated by those skilled in the art, given the present description.

In some aspects, the polymeric material can be applied to the controlled delivery device utilizing an ultrasonic spray head as described in U.S. patent application Ser. No. 10/835,530 (Anderson et al., filed Apr. 29, 2004).

In some embodiments, the surface of the body member can be pretreated prior to provision of the biodegradable polymeric coating. Any suitable surface pretreatment commonly employed in coating implantable devices can be utilized in accordance with the invention, including, for example, treatment with silane, polyurethane, parylene, and the like. For example, Parylene C (commercially available from Union Carbide Corporation), one of the three primary variants of parylene, can be used to create a polymer layer on the surface of a medical device. Parylene C is a para-xylylene containing a substituted chlorine atom, which can be coated by delivering it in a vacuum environment at low pressure as a gaseous polymerizable monomer. The monomer condenses and polymerizes on substrates at room temperature, forming a matrix on the surface of the medical device. The coating thickness can be controlled by pressure, temperature, and the amount of monomer used. The parylene coating provides an inert, non-reactive barrier.

In some embodiments, the polymeric coated composition comprises at least two layers, wherein each layer comprises the same polymeric coated composition, or different polymeric coated compositions. In one such embodiment, a first layer having either bioactive agent alone, or bioactive agent(s) together with one or more of the biodegradable polymeric materials is applied, after which one or more additional layers are applied, each with or without bioactive agent. These different layers, in turn, can cooperate in the resultant composite coating to provide an overall release profile having certain desired characteristics, and is particularly preferred for use with bioactive agents having high molecular weight. According to the invention, the composition of individual layers of the biodegradable polymeric material can include one or more bioactive agents, and one or more biodegradable polymeric materials described herein, as desired.

Preferably, the biodegradable polymeric material is applied to the body member of the controlled delivery device surface in one or more applications. The method of applying the biodegradable polymeric material to the body member is typically governed by such factors as the geometry of the device and other process considerations. The polymeric coated composition can be subsequently dried by evaporation of the solvent. The drying process can be performed at any suitable temperature, (for example, room temperature or elevated temperature), and optionally with the assistance of vacuum.

The biodegradable polymeric materials described herein can be suitably prepared for application as a coating on an implantable device. When so prepared, the biodegradable polymeric materials can be provided in any suitable form, for example, in the form of a true solution, or fluid or paste-like emulsion, mixture, dispersion, or blend. In turn, the polymeric coated composition will generally result from the removal of solvents or other volatile components and/or other physical-chemical actions (for example, heating or illumination) affecting the polymeric coated composition in situ upon the controlled delivery device surface.

The overall weight of the polymeric coated composition upon the surface of the controlled delivery device is typically not critical. The weight of the polymeric coated composition attributable to the bioactive agent can be in the range of about 1 μg to about 10 mg of bioactive agent per cm² of the surface area of the controlled delivery device. In some embodiments, the surface area can comprise all or a portion of the body member of the device. In alternative embodiments, the surface area can comprise the body member and the cap of the device. Preferably, the weight of the polymeric coated composition attributable to the bioactive agent is in the range of about 0.01 mg to about 10 mg of bioactive agent per cm² of the surface area of the controlled delivery device. This quantity of bioactive agent is generally effective to provide adequate therapeutic effect under physiological conditions. As used herein, the surface area is the macroscopic surface area of the device.

In preferred embodiments, the final thickness of the polymeric coated composition on the controlled delivery device will typically be in the range of about 0.1 μm to about 100 μm, or in the range of about 5 μm to about 60 μm. This level of coating thickness is generally effective to provide a therapeutically effective amount of bioactive agent to the implantation site under physiological conditions. The final coating thickness can be varied, and at times be outside the preferred ranges identified herein, depending upon such factors as the total amount of bioactive agent to be included in the coated composition, the type of bioactive agent, the number of bioactive agents to be included, the treatment course, the implantation site, and the like.

Thickness of the polymeric coated composition on the controlled delivery device can be assessed using any suitable techniques. For example, portions of the polymeric coated composition can be delaminated by freezing the coated controlled delivery device, for example, utilizing liquid nitrogen. The thickness at the edge of a delaminated portion can then be measured by optical microscopy. Other visualization techniques known in the art can also be utilized, such as microscopy techniques suitable for visualization of coatings having the thickness described herein of the invention.

In preferred embodiments, the controlled delivery device is sterilized utilizing common sterilization techniques, prior to implantation into the body. Sterilization can be accomplished, for example, utilizing ethylene oxide or gamma sterilization, as desired. In preferred embodiments, sterilization techniques utilized do not affect the polymeric coated composition (for example, by affecting release of the bioactive agent, stability of the coating, and the like).

Biodegradable Polymeric Material as Device Body

In some aspects, the entire implantable device is composed of the biodegradable polymeric material(s). Suitable methods for forming the implantable device from the biodegradable polymeric material have been discussed herein (such as extrusion, and the like). Once the selected biodegradable polymeric material is prepared, the polymeric material can be worked up by known methods commonly employed in the field of synthetic polymers to produce a variety of implantable articles. The articles can be shaped by conventional polymer-forming techniques, including extrusion, compression molding, injection molding, solvent casting, spin casting, and the like.

As discussed herein, any desired number (one or more) biodegradable polymeric materials can be combined to fabricate an implantable device. Thus, in some embodiments, the implantable device is composed of multiple portions that are fabricated from different biodegradable polymeric materials.

According to the invention, the controlled delivery device preferably provides the ability to deliver one or more bioactive agents in a controlled release manner. As used herein, “controlled release” refers to release of a compound (for example, a bioactive agent) into a patient's body at a desired dosage (including dosage rate and total dosage) and duration of treatment. For example, the particular composition of the biodegradable polymeric coating (including the amounts and ratios of the individual components of the biodegradable polymeric coating) can be modified to achieve a desired release profile (amount of bioactive agent released from the biodegradable polymeric coating per unit time) of the bioactive agent. While not intending to be bound by one particular theory, the release kinetics of the bioactive agent in vivo are thought to generally include both a short term (“burst”) release component, within the order of minutes to hours or less after implantation of the device, and a longer term release component, which can range from on the order of hours to days or even months of useful release. As used herein, the acceleration or deceleration of bioactive agent release can include either or both of these release kinetics components.

The desired release profile of the bioactive agent can depend upon such factors as the particular bioactive agent selected, the number of individual bioactive agents to be provided to the implantation site, the therapeutic effect to be achieved, the duration of the implant in the body, and other factors known to those skilled in the art.

The ability to provide controlled release of a bioactive agent at an implantation site can provide many advantages. For example, the controlled delivery device can be maintained at an implantation site for any desired amount of time, and the release kinetics of the bioactive agent can be adjusted to deliver the total amount of bioactive agent, at the desired rate, to achieve a desired therapeutic effect. In some embodiments, the ability to provide controlled release of bioactive agent at the implantation site allows implantation of only one device, which can be maintained in place until the desired therapeutic effect is achieved, without need to remove the device and replace the device with a new supply of bioactive agent. In some embodiments, the controlled delivery device can avoid the need for systemic application of bioactive agents, which can harm other tissues of the body. Moreover, when the entire device is fabricated of a biodegradable polymeric material, there is no need to remove the device from the patient once treatment has been completed.

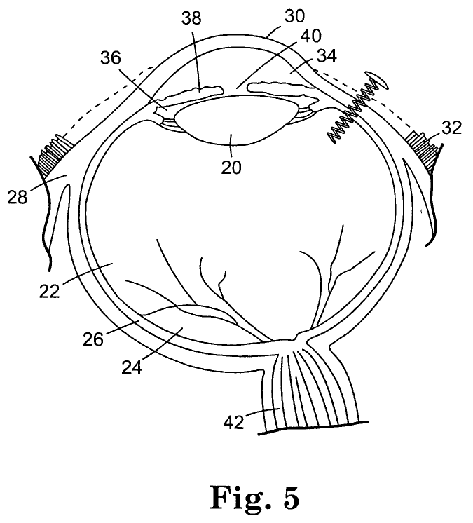

Use of the controlled delivery device can be further understood from the following discussion relating to a method for controlled release of a bioactive agent to the vitreous chamber of the eye, and with reference to FIG. 5. However, it will be understood that the principles described below can be applied to any implantation site within a patient's body.

In accordance with the invention, the controlled delivery device is fabricated, utilizing the teaching herein, in preparation for the surgical procedure. An incision in the body is made to provide access to the implantation site. For example, when used to deliver bioactive agent to the eye, a sclerotomy is created for insertion of the controlled delivery device. Conventional techniques can be used for the creation of the sclerotomy. Such techniques include the dissection of the conjunctiva 32 and the creation of pars plana scleral incisions through the sclera 28. The dissection of the conjunctiva 32 typically involves pulling back the conjunctiva 32 about the eye so as to expose large areas of the sclera 28, and the clipping or securing of the conjunctiva 32 in that pulled back state (the normal position of the conjunctiva is shown in phantom). In other words, the sclera 28 is exposed only in the areas where the pars plana scleral incisions are to be made. If additional surgical instruments are used in the procedure (for example, for placement of the device at the implantation site), such instruments are then passed through these incisions. Thus, the incisions should be made large enough to accommodate the instruments required for the procedure.

FIG. 5 illustrates a cross-sectional view of the eye. Beginning from the exterior of the eye, the structure of the eye includes the iris 38 that surrounds the pupil 40. The iris 38 is a circular muscle that controls the size of the pupil 40 to control the amount of light allowed to enter the eye. A transparent external surface, the cornea 30, covers both the pupil 40 and the iris 38. Continuous with the cornea 30, and forming part of the supporting wall of the eyeball, is the sclera 28 (the white of the eye). The conjunctiva 32 is a clear mucous membrane covering the sclera 28. Within the eye is the lens 20, which is a transparent body located behind the iris 38. The lens 20 is suspended by ligaments attached to the anterior portion of the ciliary body (not illustrated in the figures). The contraction or relaxation of these ligaments as a consequence of ciliary muscle actions changes the shape of the lens 20, a process called accommodation, and allows a sharp image to be formed on the retina 24. Light rays are focused through the transparent cornea 30 and lens 20 upon the retina 24. The central point for image focus (the visual axis) in the human retina is the fovea (not shown in the figures). The optic nerve 42 is located opposite the lens.

There are three different layers of the eye, the external layer, formed by the sclera 28 and cornea 30; the intermediate layer, which is divided into two parts, namely the anterior (iris 38 and ciliary body) and posterior (the choroid 26); and the internal layer, or the sensory part of the eye, formed by the retina 24. The lens 20 divides the eye into the anterior segment (in front of the lens) and the posterior segment (behind the lens). More specifically, the eye is composed of three chambers of fluid: the anterior chamber 34 (between the cornea 30 and the iris 38), the posterior chamber 36 (between the iris 38 and the lens 20), and the vitreous chamber 22 (between the lens 20 and the retina 24). The anterior chamber 34 and posterior chamber 36 are filled with aqueous humor whereas the vitreous chamber 22 is filled with a more viscous fluid, the vitreous humor.

Alternatively, the creation of the sclerotomy can be accomplished by use of an alignment device and method, such as that described in U.S. patent application Ser. No. 09/523,767, that enables sutureless surgical methods and devices thereof. In particular, such methods and devices do not require the use of sutures to seal the openings through which instruments are inserted. The alignment devices are inserted through the conjunctiva and sclera to form one or more entry apertures. Preferably, the alignment devices are metal or polyimide cannulas through which the surgical instruments used in the procedure are inserted into the eye.

In further embodiments, the device can be implanted directly through a self-starting transconjunctival trans-scleral “needle stick.” For example, the body member of the device can include a sharp tip. According to this embodiment, the sharp distal tip can be utilized to pierce the body and thereby create the incision site and access to the implantation site. In this case, no conjunctival surgery or extraneous alignment device is necessary.

In further embodiments, the conjunctival tissue can be dissected to expose a portion of the pars plana region, and a needlestick can be made into the sclera in the exposed region. A self-starting device that includes a sharp tip is then inserted through the pars plana at the site of the needlestick, and the device is inserted through the sclera until the cap of the device abuts the sclera. In some preferred embodiments, the needlestick is smaller than the diameter of the body member of the implantable device (for example, a 30-gauge needlestick can be used with an implantable device having a body member with a diameter of 0.5 mm or less). The conjunctival tissue is then pulled over the cap, to provide a flap or “seal” over the device, thus minimizing irritation of the implantation site, foreign body sensation, and the like. Optionally, the conjunctival tissue can be further secured by a suture (in preferred embodiments, a biodegradable suture).

In some embodiments, it can be preferable to create an incision site that is slightly larger than the dimensions of the proximal portion of the body member. For example, when the device includes a cap and is implanted into the eye, it can be preferable to create an incision that is larger than the largest diameter of the cap, such that the cap sits below the outer surface of the sclera. For example, a partial incision in the sclera can be made to create a scleral flap. Once the device has been implanted, and the cap is placed so that it abuts the incision site, the scleral flap can be folded back over the device, thus providing a covering over the cap. Alternatively, when the proximal end of the body member does not include a cap, a flap-like cover can still be utilized to cover the proximal end of the device, in accordance with the description above. Preferably, these embodiments minimize the contact of the proximal end (for example, the cap) of the device with other body tissues, thereby reducing such risks as irritation of body tissues, and/or translation of movement of the eye to the device, thereby potentially damaging eye tissues. This can provide one or more advantages, such as reduced tendency for movement of the eye to be translated to the controlled delivery device, since the proximal end of the device will not be sitting at the surface of the eye and thus in contact with other body tissues; and reduced irritation of surrounding tissues.

The body member is then inserted into the eye. For example, in embodiments wherein the body member has a coil shape, the body member is inserted into the eye by rotating or twisting the body member into the eye until the cap abuts the outer surface of the eye.

When implanted into the eye, it is desirable to limit the length of controlled delivery devices to prevent the controlled delivery device from entering the central visual field. If the implant enters the central visual field, this can result in blind spots in the patient's vision and can increase the risk of damage to the retinal tissue and lens capsule. Thus, for example, when the controlled delivery device is inserted at the pars plana, the distance from the implantation site on the pars plana to the central visual field is preferably less than about 1 cm.

Optionally, after the device is implanted into the eye, the cap can then be sutured or otherwise secured to the sclera to maintain the controlled delivery device in place. In preferred embodiments, no further manipulation of the device is required for delivery of one or more bioactive agents to the interior of the eye. The conjunctiva can be adjusted to cover the cap of the device, when desired, and the surgical procedure is completed.

In other embodiments, when a lumen is included in the device for delivery of one or more additional substances to the interior of the eye, further steps can be included as follows. If a cover is used to close the port(s), it is removed at this time, and if used, a collar for providing a snug fit about the injection mechanism (such as a syringe) is provided. The injection mechanism is then connected with the port(s) for injection of one or more substances to the controlled delivery device. If the port(s) are composed of a self-sealing material through which the needle of an injection mechanism can be inserted and which seals off automatically when the injection mechanism is removed, the injection mechanism is simply inserted through the port and the substance injected. Following injection, the conjunctiva can be adjusted to cover the cap of the device, if desired.

The controlled delivery device of the invention can be used to deliver one or more bioactive agents to the eye for the treatment of a variety of ocular conditions such as, for example, retinal detachment; occlusions; proliferative retinopathy; proliferative vitreoretinopathy; diabetic retinopathy; inflammations such as uveitis, choroiditis, and retinitis; degenerative disease (such as age-related macular degeneration, also referred to as AMD); vascular diseases; and various tumors including neoplasms. In yet further embodiments, the controlled delivery device can be used post-operatively, for example, as a treatment to reduce or avoid potential complications that can arise from ocular surgery. In one such embodiment, the controlled delivery device can be provided to a patient after cataract surgical procedures, to assist in managing (for example, reducing or avoiding) post-operative inflammation.

In some applications, additives can further be included with the bioactive agent and/or additional substance to be delivered to the implantation site. Examples of suitable additives include, but are not limited to, water, saline, dextrose, carriers, preservatives, stabilizing agents, wetting agents, emulsifying agents, excipients, and the like.

According to the invention, once the desired treatment course is completed, the implantable device can degrade and optionally be absorbed by the body. Thus, removal of the implantable device from the body is not required. In preferred aspects, this can further reduce patient risk of infection and other complications, since the amount of surgical intervention is reduced. Obviously, if removal of the device is desired for any reason, the portions of the implantable device remaining within the patient can be removed at the desired point in time. In some embodiments, degradation of the polymeric material of the device takes place over the treatment course, with the result that little significant amount of polymeric material remains at the completion of the treatment course. When less than the total implantable device is fabricated of a biodegradable polymeric material, the components of the device that are fabricated from the degradable material can be significantly degraded and/or absorbed by the body around the completion of the treatment course.

The suitability of particular polymeric coated compositions for in vivo use can be determined by one or more of a variety of methods, including the Durability Test and Elution Assay.

Sample Preparation

One-millimeter diameter stainless steel wires (for example, 316 L grade) are cut into desired lengths. The wire segments are treated with a Parylene C coating composition (Union Carbide Corporation), as described herein. The wire segments are weighed on a micro-balance.

Biodegradable polymeric coatings are prepared at a range of concentrations in an appropriate solvent, in the manner described herein. The coating mixtures are applied to respective wires, or portions thereof, by dipping or spraying, and the coated wires are allowed to dry by solvent evaporation. The coated wires are then re-weighed. From this weight, the mass of the coatings can be calculated, which in turn permits the mass of the coated polymer(s) and bioactive agent(s) to be determined.

The durability of the polymeric coated composition can be determined in the following manner.

Durability Test

For the Durability Test, coated devices are prepared as described above. The coated devices are mounted to an insertion tool that firmly engages the cap of the device while avoiding mechanical contact with the coated portion of the device. The devices can include a distal sharp tip that is utilized to pass through the conjunctiva and sclera and into the interior of the eye. Cadaveric porcine eyes can be utilized, and the distal sharp tip is utilized to place the devices into the eye until the cap of the device is flush with the sclera.

After implantation, the coated devices are immediately removed, utilizing the insertion device used for implantation. Devices are carefully cleaned without the use of solvents (deionized water is used to remove any tissue adhering to the device surface). The devices are then analyzed for surface coating defects (such as delamination of the coating) under light microscopy.

Elution Assay

Any suitable Elution Assay can be used to determine the extent and/or rate of bioactive agent release from the polymeric coated composition under physiological conditions. In general, it is desirable that less than 50% of the total quantity of the drug to be released is released in the first 24 hours after introduction into physiological conditions. It is frequently desirable for quantities of bioactive agent to be released for a duration of at least 30 days.

In one exemplary Elution Assay, phosphate buffered saline (PBS, 10 mM phosphate, 150 mM NaCl, pH 7.4, aqueous solution) is pipetted in an amount of 3 ml to 10 ml into an amber vial with a Teflon™ lined cap. A wire or coil treated with the biodegradable polymeric coating is immersed into the PBS. A stir bar is placed into the vial and the cap is screwed tightly onto the vial. The PBS is stirred with the use of a stir plate, and the temperature of the PBS is maintained at 37° C. with the use of a water bath. The sampling times are chosen based upon the expected or desired elution rate. At the sampling time point, the wire or coil is removed from the vial and placed into a new vial containing fresh PBS. A UV/V is spectrophotometer can be used to determine the concentration of the bioactive agent in the PBS solution that previously contained the wire or coil treated with the biodegradable polymeric coating. The cumulative amount of bioactive agent eluted versus time can be plotted to obtain an elution profile.

At the conclusion of the Elution Assay, the wire or coil is washed with water, dried and re-weighed. Correlation between the percent bioactive agent eluted and the percent weight loss of the polymeric coated composition can be verified. Weight loss of the polymeric coated composition can also be due to degradation of the polymer.

When desired, the coating can also be evaluated by measuring the coating thickness (for example, using a Minitest 4100 thickness gauge), and the coating quality (such as roughness, smoothness, evenness, and the like) can be analyzed by SEM analysis.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

We claim:

1. A sustained release implant configured to reside in a posterior segment of an eye, the implant comprising one or more bioactive agents and a biodegradable polymeric material in a solid form, wherein the implant is configured to provide sustained release of the bioactive agent into the posterior segment of the eye, and wherein the biodegradable polymeric material comprises a random block copolymer having a formula: 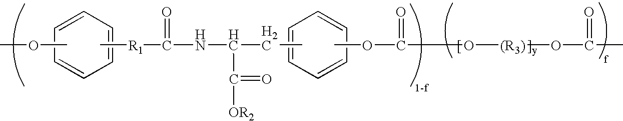
wherein R₁ is —CH═CH— or (—CH₂—)ⱼ, in which j is zero or an integer from one to eight; R₂ is selected from straight and branched alkyl and alkylaryl groups containing up to 18 carbon atoms and optionally containing at least one ether linkage, and derivatives of biologically and pharmaceutically active compounds covalently bonded to the copolymer;
each R₃ is independently selected from alkylene groups containing 1 to 4 carbon atoms;
y is between 5 and about 3000; and
f is the percent molar fraction of alkylene oxide in the copolymer, and is in the range of about 1 to about 99 mole percent.

2. The implant according to claim 1 wherein the implant is configured for placement in a subretinal area of the eye.

3. The implant according to claim 1 wherein the implant is provided in a linear configuration for placement in the posterior segment of the eye.

4. The implant according to claim 3 wherein the linear configuration is a filament, rod, wire, or film.

5. The implant according to claim 1 wherein the implant is provided in a rounded configuration for placement in the posterior segment of the eye.

6. The implant according to claim 5 wherein the rounded configuration is disc-shaped, bead-shaped, or oblong rounded shaped.

7. The implant according to claim 1 wherein R₁ is —CH₂—CH₂—.

8. The implant according to claim 1 wherein R₂ is a straight-chained alkyl group selected from ethyl, butyl, hexyl, and octyl groups.

9. The implant according to claim 1 wherein each R₃ is ethylene.

10. The implant according to claim 1 wherein y is in the range of 20 to 200.

11. The implant according to claim 1 wherein f is in the range of 5 to 95 mole percent.

12. The implant according to claim 1 wherein the biodegradable polymeric material and one or more bioactive agents are provided as a coating on a surface of the implant.

13. The implant according to claim 12 wherein the implant is fabricated of a body member comprising nondegradable material.

14. The implant according to claim 12 wherein the coating is provided on a portion of the implant surface.

15. The implant according to claim 14 wherein the coating is provided on an intermediate portion of the implant surface.

16. The implant according to claim 12 wherein the coating is provided with a stepped coating thickness, such that the coating thickness decreases towards a proximal and/or distal end of the implant.

17. The implant according to claim 1 wherein the bioactive agent is selected from antiproliferative agent, anti-inflammatory agent, anti-angiogenic agent, antibiotic, neurotrophic factor, or a combination of any two or more of these.

18. The implant according to claim 1 wherein the bioactive agent is admixed with the biodegradable polymeric material.

19. The implant according to claim 1 wherein R₂ is one or more derivatives of biologically and pharmaceutically active compounds covalently bonded to the copolymer.

20. The implant according to claim 1 wherein the implant comprises:
a nonlinear body member having a direction of extension, a longitudinal axis along the direction of extension, and a proximal end and a distal end,
wherein at least a portion of the body member deviates from the direction of extension, and wherein the body member includes the one or more bioactive agents, and the polymer matrix comprises a biodegradable polymer comprising an amino acid-derived polycarbonate or polyarylate.

21. The implant according to claim 20 wherein the body member is coil-shaped.

22. The implant according to claim 20 wherein a cap is positioned at the proximal end of the body member.

23. The implant according to claim 20 wherein the body member includes a lumen.

24. The implant according to claim 20 wherein the biodegradable polymer material and one or more bioactive agents are provided as a coating on a surface of the implant.

25. The implant according to claim 24 wherein the implant is fabricated of a body member comprising nondegradable material.

26. The implant according to claim 24 wherein the coating is provided on a portion of the implant surface.

27. The implant according to claim 26 wherein the coating is provided on an intermediate portion of the body member.

28. The implant according to claim 20 wherein the coating is provided with a stepped coating thickness, such that the coating thickness decreases towards the proximal and/or distal end of the body member.

29. The implant according to claim 20 wherein the bioactive agent is selected from antiproliferative agent, anti-inflammatory agent, anti-angiogenic agent, antibiotic, neurotrophic factor, or a combination of any two or more of these.

30. The implant according to claim 20 wherein the bioactive agent is admixed with the biodegradable polymeric material.

31. The implant according to claim 20 wherein the device is removable from the eye after a desired treatment.

32. A method of making a device for controlled release of bioactive agent to a posterior segment of an eye, the method comprising steps of:
(a) providing a biodegradable polymer comprising a random block copolymer having a formula: 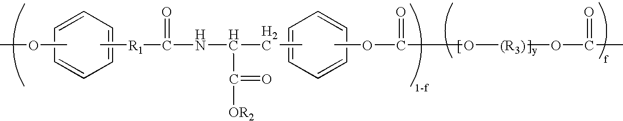
wherein R₁ is —CH═CH— or (—CH₂—)ⱼ, in which j is zero or an integer from one to eight;
R₂ is selected from straight and branched alkyl and alkylaryl groups containing up to 18 carbon atoms and optionally containing at least one ether linkage, and derivatives of biologically and pharmaceutically active compounds covalently bonded to the copolymer;
each R₃ is independently selected from alkylene groups containing 1 to 4 carbon atoms;
y is between 5 and about 3000; and
f is the percent molar fraction of alkylene oxide in the copolymer, and is in the range of about 1 to about 99 mole percent;
(b) combining the random block copolymer with one or more bioactive agents; and
(c) forming at least a portion of a solid implant from the random block copolymer with bioactive agent, wherein the solid implant is configured for placement in ocular tissues within the posterior segment of the eye.

33. The method according to claim 32 wherein the step (c) comprises forming the random block copolymer with bioactive agent into a filament, rod, film, disc, bead, or oblong rounded shaped implant for placement in a subretinal area of the eye.

34. The method according to claim 32 wherein the step (c) comprises providing a body member, and providing the random block copolymer with one or more bioactive agents as a coating to a surface of the body member, wherein the body member is a filament, rod, film, disc, bead, or oblong rounded shaped implant for placement in a subretinal area of the eye.

35. The method according to claim 34 wherein the body member is formed of nondegradable material.

36. The method according to claim 32 wherein the step (c) comprises forming the random block copolymer with bioactive agent into a nonlinear body member having a direction of extension, a longitudinal axis along the direction of extension, and a proximal end and a distal end, wherein at least a portion of the body member deviates from the direction of extension, the implant configured for intraocular placement within an eye.

37. The method according to claim 32 wherein the step (c) comprises providing a body member, and providing the random block copolymer with bioactive agent as a coating to a surface of the body member, wherein the body member is a nonlinear body member having a direction of extension, a longitudinal axis along the direction of extension, and a proximal end and a distal end, wherein at least a portion of the body member deviates from the direction of extension, the implant configured for intraocular placement within an eye.

38. The method according to claim 37 wherein the body member is formed of nondegradable material.

39. A method for delivery of bioactive agent to ocular tissue within a patient in a controlled manner, the method comprising steps of providing an implant in a posterior segment of the patient's eye, the implant comprising one or more bioactive agents and a biodegradable polymeric material in a solid form, wherein the implant is configured to provide sustained release of the bioactive agent into the posterior segment of the eye, and wherein the biodegradable polymeric material comprises a random block copolymer having a formula: 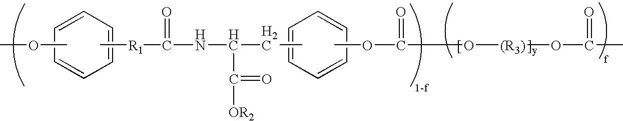
wherein R₁ is —CH═CH— or (—CH₂—)ⱼ, in which j is zero or an integer from one to eight;
R₂ is selected from straight and branched alkyl and alkylaryl groups containing up to 18 carbon atoms and optionally containing at least one ether linkage, and derivatives of biologically and pharmaceutically active compounds covalently bonded to the copolymer;
each R₃ is independently selected from alkylene groups containing 1 to 4 carbon atoms;
y is between 5 and about 3000; and
f is the percent molar fraction of alkylene oxide in the copolymer, and is in the range of about 1 to about 99 mole percent.
